(19) United States
(12) Patent Application Publication
(10) Pub. No.: US 2022/0170915 A1
(43) Pub. Date: Jun. 2, 2022

(54) DIGITAL HEALTH ECOSYSTEM

(71) Applicant: JUNO DIAGNOSTICS, INC., San Diego, CA (US)

(72) Inventors: Dirk VAN DEN BOOM, Encinitas, CA (US); Mathias EHRICH, San Diego, CA (US); Jim CHAUVAPUN, San Diego, CA (US); Eugene SHTEYN, Cupertino, CA (US)

(21) Appl. No.: 17/598,052

(22) PCT Filed: Mar. 26, 2020
(86) PCT No.: PCT/US2020/025022

§ 371 Date: Sep. 24, 2021

Related U.S. Application Data
(60) Provisional application No. 62/824,765, filed on Mar. 27, 2019.

(51) Int. Cl. G01N 33/53; G16H 20/00

(52) U.S. Cl. CPC G01N 33/53; G16H 20/00

(57) ABSTRACT

Described are computer-implemented methods, systems, and platforms for monitoring biological data of a subject, and providing real-time recommendations to the user related to a change in the subjects health status. Disclosed herein are sampling devices in communication with at least one computer processor of the systems and platforms described herein, which sampling devices are configured to measure the level, presence, or absence of a one or more biomarkers indicative of the subjects health status.

### CROSS-REFERENCE

This international application claims benefit to U.S. Provisional Application Ser. No. 62/824,765, filed Mar. 27, 2019, which is incorporated herein by reference in its entirety.

### SUMMARY

Aspects disclosed herein comprise computer-implemented platforms comprising: (a) a sampling device configured to: (i) receive a biologic sample from a user; (ii) analyze the biologic sample to detect a quantity, a presence, or both of an analyte; and (iii) a mobile processor configured to provide a mobile application, the mobile application comprising: (b) a user sourced information module receiving user biological data; and (c) a data processor configured to provide a recommendation application, the recommendation application comprising: (i) a reception module receiving the user biological data and at least one of the quantity of the analyte or the presence of the analyte; (ii) a recommendation generation module determining a recommendation based on the user biological data and at least one of the quantity of the analyte or the presence of the analyte; and (ii) a transmission module transmitting the recommendation to the mobile processor; wherein at least one of the mobile processor and the data processor are further configured to provide a sample module receiving the quantity of the analyte, the presence of the analyte, or both. In some embodiments, at least one of the user sourced information module further receive an externally sourced data. In some embodiments, the externally sourced data comprises a website, a video, a document file, a medical record, a pharmacy record, a medication history, a health insurance information, a subscription information, metabolic activity data, physical activity data, heart rate data, blood pressure data, blood oxygen levels, metabolite data, sleep data, augmentation data, genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions, or any combination thereof. In some embodiments, the recommendation application further comprises a database having a plurality of recommendation templates. In some embodiments, the recommendation application further comprises a template selection module selecting at least one recommendation template from the plurality of recommendation templates based on the user biological data and at least one of the quantity of the analyte or the presence of the analyte. In some embodiments, the recommendation generation module further determines the recommendation based on the at least one selected recommendation templates. In some embodiments, the at least one recommendation template comprises a trigger, a rule, or both. In some embodiments, the recommendation is further based on the trigger, the rule, or both. In some embodiments, the at least one recommendation template is a pre-defined template or a custom template. In some embodiments, the at least one recommendation template is determined by a machine-learning algorithm. In some embodiments, the recommendation application further comprises an access control module confirming an access of the recommendation to the user, a third party, or both. In some embodiments, the transmission module transmits the recommendation to the user, the one or more service agents, or both based on the confirmation of access. In some embodiments, the recommendation generation module determines the recommendation by a machine learning algorithm. In some embodiments, the user biological data comprises a weight, blood pressure, height, heart rate, blood oxygen levels, food intake, nutritional history, activity history, sleep history, geolocation, travel information, body temperature, step count, body fat percentage, an emergency contact, a family contact, a friend contact, genetic data, genomic data, epigenetic information, microbiome information, proteomic and transcriptomic information, immune repertoire information, pharmacogenetics, or drug-drug interactions, or any combination thereof. In some embodiments, the recommendation comprises a fitness recommendation, nutrition recommendation, mental health recommendation, a recommendation for further testing, or any combination thereof. In some embodiments, the recommendation for further testing relates to the sampling device, or additional testing device. In some embodiments, the sampling device comprises: (a) a sample purifier for removing a cell from a biological fluid sample to produce a cell-depleted sample; and (b) at least one of a detection reagent and a signal detector for detecting a plurality of cell-free DNA fragments in the cell-depleted sample. In some embodiments, the sample purifier comprises a filter, and wherein the filter has a pore size of about 0.05 microns to about 2 microns. In some embodiments, the filter is a vertical filter. In some embodiments, the sample purifier comprises a binding moiety selected from an antibody, antigen binding antibody fragment, a ligand, a receptor, a peptide, a small molecule, and a combination thereof. In some embodiments, the binding moiety is capable of binding an extracellular vesicle. In some embodiments, the at least one nucleic acid amplification reagent comprises an isothermal amplification reagent. In some embodiments, the signal detector is a lateral flow strip. In some embodiments, the data processor and the sampling device are contained in a single housing. In some embodiments, the sampling device is capable of detecting the plurality of biomarkers in the cell-depleted sample within about five minutes to about twenty minutes of receiving the biological fluid. In some embodiments, the analyte is selected from a hormone, a lipid, a carbohydrate, metabolite, drug metabolite a protein, a peptide, DNA, RNA, an epigenetic marker, a pathogen, a microbe or a portion thereof. In some embodiments, the sample comprises blood, urine, interstitial fluid, tear fluid, tissue, hair, or sweat.

Aspects disclosed herein provide computer-implemented methods comprising: (a) receiving, by a sampling device, a biologic sample from the user; (b) analyzing, by the sampling device, the biologic sample to detect a quantity, a presence, or both of an analyte; and (c) receiving, by a mobile processor, a user biological data; (d) receiving, by the mobile processor or a data processor, the quantity of the analyte, the presence of the analyte, or both; (e) receiving, by the data processor, the user biological data and at least one of the quantity of the analyte or the presence of the analyte; (f) generating, by the data processor, a recommendation based on the user biological data and at least one of the quantity of the analyte or the presence of the analyte; and (g) transmitting the recommendation to the mobile processor. In some embodiments, methods further comprise receiving, by at least one of the user sourced information module an externally sourced data. In some embodiments, the externally sourced data comprises a website, a video, a document file, a medical record, a pharmacy record, a medication history, a health insurance information, a subscription information, metabolic activity data, physical activity data, heart rate data, blood pressure data, metabolite data, sleep data, augmentation data, genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions, or any combination thereof. In some embodiments, methods further comprise storing, in a database a plurality of recommendation templates. In some embodiments, methods further comprise selecting, by the data processor at least one recommendation template from the plurality of recommendation templates based on the user biological data and at least one of the quantity of the analyte or the presence of the analyte. In some embodiments, the user biological data comprises a weight, blood pressure, height, heart rate, blood oxygen levels, food intake, nutritional history, activity history, sleep history, geolocation, travel information, body temperature, step count, body fat percentage, an emergency contact, a family contact, a friend contact, genetic data, genomic data, epigenetic information, microbiome information, proteomic and transcriptomic information, immune repertoire information, pharmacogenetics, or drug-drug interactions, blood oxygen levels, travel information, or any combination thereof. In some embodiments, methods further comprise determining, by the data processor, the recommendation based on the at least one selected recommendation templates. In some embodiments, the at least one selected recommendation template comprises a trigger, a rule, or both. In some embodiments, methods further comprise determining, by the data processor, the recommendation based on the trigger, the rule, or both. In some embodiments, the at least one selected recommendation template is a pre-defined template or a custom template. In some embodiments, the at least one selected recommendation template is determined by a machine-learning algorithm. In some embodiments, methods further comprise confirming, by the data processor, an access of the recommendation to the user, a third party, or both. In some embodiments, methods further comprise transmitting, by the data processor, of the recommendation to the user, the one or more service agents, or both is based on the confirmation of access. In some embodiments, methods further comprise transmitting, by the mobile processor, the recommendation to a service agent. In some embodiments, the transmission is based on the confirmation of access. In some embodiments, determining, by the data processor, the recommendation is performed by a machine learning algorithm. In some embodiments, the analyte is selected from a hormone, a lipid, a carbohydrate, metabolite, a drug metabolite, a protein, a peptide, DNA, RNA, an epigenetic marker, a pathogen, a microbe or a portion thereof. In some embodiments, the sample comprises blood, urine, interstitial fluid, tear fluid, tissue, hair, or sweat.

Aspects disclosed herein provide computer-readable storage medium comprising instructions executable by at least one processor, the instructions disclosed herein.

Aspects disclosed herein provide a computer-implemented platform comprising: (a) a sampling device configured to receive a biologic sample from a user; analyze the biologic sample to detect a quantity, a presence, or both of an analyte; and (b) a mobile processor configured to provide a mobile application, the mobile application comprising: (i) a user sourced information module receiving a user biological data; and (ii) a data processor configured to provide a recommendation application, the recommendation application comprising: (1) a reception module receiving the user biological data and at least one of the quantity of the analyte or the presence of the analyte; (2) a recommendation generation module determining a recommendation based on the user biological data and at least one of the quantity of the analyte or the presence of the analyte; (3) and a transmission module transmitting the recommendation to the mobile processor; wherein at least one of the mobile processor and the data processor are further configured to provide a sample module receiving the quantity of the analyte, the presence of the analyte, or both. In some embodiments, at least one of the user sourced information module or the reception module further receive an externally sourced data. In some embodiments, the externally sourced data comprises a website, a video, a document file, a medical record, a pharmacy record, a medication history, a health insurance information, a subscription information, metabolic activity data, physical activity data, heart rate data, blood pressure data, metabolite data, sleep data, augmentation data (e.g., from augmented realty or virtual reality applications), genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions, or any combination thereof. In some embodiments, the recommendation application further comprises a database having a plurality of recommendation templates. In some embodiments, the recommendation application further comprises a template selection module selecting at least one recommendation template from the plurality of recommendation templates based on the user biological data and at least one of the quantity of the analyte or the presence of the analyte. In some embodiments, the recommendation generation module further determines the recommendation based on the one or more selected recommendation templates. In some embodiments, the template comprises a trigger, a rule, or both. In some embodiments, the recommendation is further based on the trigger, the rule, or both. In some embodiments, the template is a pre-defined template or a custom template. In some embodiments, the template is determined by a machine-learning algorithm. In some embodiments, the recommendation application further comprises an access control module confirming an access of the recommendation to the user, a third party, or both. In some embodiments, the transmission module transmits the recommendation to the user, the one or more service agents, or both based on the confirmation of access. In some embodiments, the transmission module transmits the recommendation to a service agent. In some embodiments, the transmission module transmits the recommendation to the user, the one or more service agents, or both based on the confirmation of access. In some embodiments, the recommendation generation module determines the recommendation by a machine learning algorithm. In some embodiments, the analyte comprises a predisposition biomarker, diagnostic biomarker, prognostic biomarker, predictive biomarker, DNA, RNA, protein, metabolite, circulating cell-free nucleic acid, or any combination thereof. In some embodiments, the user biological data comprises a weight, blood pressure, height, heart rate, food intake, nutritional history, activity history, sleep history, geolocation, body temperature, step count, body fat percentage, an emergency contact, a family contact, a friend contact, genetic data, genomic data, epigenetic information, microbiome information, proteomic and transcriptomic information, immune repertoire information, pharmacogenetics, blood oxygen levels, travel information, or drug-drug interactions, or any combination thereof. In some embodiments, the recommendation comprises a fitness recommendation, nutrition recommendation, mental health recommendation, a recommendation for further testing, or any combination thereof. In some embodiments, the recommendation for further testing relates to the sampling device, or additional testing device. In some embodiments, the sampling device comprises: (a) a sample purifier for removing a cell from a biological fluid sample to produce a cell-depleted sample; and (b) at least one of a detection reagent and a signal detector for detecting a plurality of cell-free DNA fragments in the cell-depleted sample. In some embodiments, a first sequence is present on a first cell-free DNA fragment of the plurality of cell-free DNA fragments and a second sequence is present on a second cell-free DNA fragment of the plurality of cell-free DNA fragments, and wherein the first sequence is at least 80% identical to the second sequence. In some embodiments, the sampling device comprises at least one nucleic acid amplification reagent and a single pair of primers capable of amplifying the first sequence and the second sequence. In some embodiments, at least one of the first sequence and the second sequence is repeated at least twice in a genome of a user. In some embodiments, the first sequence and the second sequence are each at least 10 nucleotides in length. In some embodiments, the first sequence is on a first chromosome and the second sequence is on a second chromosome. In some embodiments, the first sequence and the second sequence are on the same chromosome but separated by at least 1 nucleotide. In some embodiments, the first sequence and the second sequence are in functional linkage. In some embodiments, the sample purifier comprises a filter, and wherein the filter has a pore size of about 0.05 microns to about 2 microns. In some embodiments, the filter is a vertical filter. In some embodiments, the sample purifier comprises a binding moiety selected from an antibody, antigen binding antibody fragment, a ligand, a receptor, a peptide, a small molecule, and a combination thereof. In some embodiments, the binding moiety is capable of binding an extracellular vesicle. In some embodiments, the at least one nucleic acid amplification reagent comprises an isothermal amplification reagent. In some embodiments, the signal detector is a lateral flow strip. In some embodiments, the platform is contained in a single housing. In some embodiments, the platform operates at room temperature. In some embodiments, the sampling device is capable of detecting the plurality of biomarkers in the cell-depleted sample within about five minutes to about twenty minutes of receiving the biological fluid. In some embodiments, the platform further comprises a transdermal puncture platform. In some embodiments, the analyte is selected from a hormone, a lipid, a carbohydrate, a metabolite, a drug metabolite, a protein, a peptide, DNA, RNA, an epigenetic marker, a pathogen, a microbe or a portion thereof. In some embodiments, the sample comprises blood, urine, interstitial fluid, tear fluid, tissue, hair, or sweat.

Aspects provided herein provide computer-implemented methods comprising: (a) receiving, by a sampling device, a biologic sample from the user; (b) analyzing, by the sampling device, the biologic sample to detect a quantity, a presence, or both of an analyte; (c) receiving, by a mobile processor, a user biological data; (d) receiving, by the mobile processor or a data processor, the quantity of the analyte, the presence of the analyte, or both; (e) receiving, by the data processor, the user biological data and at least one of the quantity of the analyte or the presence of the analyte; (f) generating, by the data processor, a recommendation based on the user biological data and at least one of the quantity of the analyte or the presence of the analyte; and (g) transmitting the recommendation to the mobile processor. In some embodiments, the method further comprises receiving, by at least one of the user sourced information module or the reception module, an externally sourced data. In some embodiments, the externally sourced data comprises a website, a video, a document file, a medical record, a pharmacy record, a medication history, a health insurance information, a subscription information, metabolic activity data, physical activity data, heart rate data, blood pressure data, metabolite data, sleep data, augmentation data (e.g., from augmented realty or virtual reality applications), genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions, or any combination thereof. In some embodiments the method further comprises storing, in a database a plurality of recommendation templates. In some embodiments the method further comprises selecting, by the data processor at least one recommendation template from the plurality of recommendation templates based on the user biological data and at least one of the quantity of the analyte or the presence of the analyte. In some embodiments the method further comprises determining, by the data processor, the recommendation based on the one or more selected recommendation templates. In some embodiments, the template comprises a trigger, a rule, or both. In some embodiments the method further comprises determining, by the data processor, the recommendation based on the trigger, the rule, or both. In some embodiments, the template is a pre-defined template or a custom template. In some embodiments, the template is determined by a machine-learning algorithm. In some embodiments the method further comprises confirming, by the data processor, an access of the recommendation to the user, a third party, or both. In some embodiments, transmitting, by the data processor, of the recommendation to the user, the one or more service agents, or both is based on the confirmation of access. In some embodiments the method further comprises transmitting, by the mobile processor, the recommendation to a service agent. In some embodiments, the transmission is based on the confirmation of access. In some embodiments, determining, by the data processor, the recommendation is performed by a machine learning algorithm. In some embodiments, the analyte comprises a predisposition biomarker, diagnostic biomarker, prognostic biomarker, predictive biomarker, DNA, fetal circulating cell-free DNA, prostate specific antigen, or any combination thereof. In some embodiments, the user biological data comprises a weight, blood pressure, height, heart rate, food intake, nutritional history, activity history, sleep history, geolocation, body temperature, step count, body fat percentage, an emergency contact, a family contact, a friend contact, genetic data, genomic data, epigenetic information, microbiome information, proteomic and transcriptomic information, immune repertoire information, pharmacogenetics, or blood oxygen levels, travel information, drug-drug interactions, or any combination thereof. In some embodiments, the recommendation comprises a fitness recommendation, nutrition recommendation, mental health recommendation, a recommendation for further testing, or any combination thereof. In some embodiments, the recommendation for further testing relates to the sampling device, or additional testing device. In some embodiments, the analyte is selected from a hormone, a lipid, a carbohydrate, a metabolite, a drug metabolite, a protein, a peptide, DNA, RNA, an epigenetic marker, a pathogen, a microbe or a portion thereof. In some embodiments, the sample comprises blood, urine, interstitial fluid, tear fluid, tissue, hair, or sweat.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a non-limiting example of a recommendation platform, in accordance with some embodiments;

FIG. 2 shows a non-limiting example of a machine-learning based template generator, in accordance with some embodiments;

FIG. 3 shows a non-limiting example of analyzing the biologic sample to detect a quantity, a presence, or both of an analyte, in accordance with some embodiments;

FIG. 4 shows a first non-limiting example of a mobile application, in accordance with some embodiments;

FIG. 5A shows a second non-limiting example of a mobile application, in accordance with some embodiments;

FIG. 5B shows a third non-limiting example of a mobile application, in accordance with some embodiments;

FIG. 5C shows a fourth non-limiting example of a mobile application, in accordance with some embodiments;

FIG. 5D shows a fifth non-limiting example of a mobile application, in accordance with some embodiments;

FIG. 5E shows a sixth non-limiting example of a mobile application, in accordance with some embodiments;

FIG. 5F shows a seventh non-limiting example of a mobile application, in accordance with some embodiments;

FIG. 5G shows a eighth non-limiting example of a mobile application, in accordance with some embodiments;

FIG. 6 shows a non-limiting example of a computing device; in this case, a device with one or more processors, memory, storage, and a network interface, in accordance with some embodiments;

FIG. 7 shows a non-limiting example of a web/mobile application provision system; in this case, a system providing browser-based and/or native mobile user interfaces, in accordance with some embodiments;

FIG. 8 shows a non-limiting example of a cloud-based web/mobile application provision system; in this case, a system comprising an elastically load balanced, auto-scaling web server and application server resources as well synchronously replicated databases, in accordance with some embodiments;

FIG. 9 shows a non-limiting example of template paths, in accordance with some embodiments; and

FIG. 10 shows a non-limiting example of a platform for monitoring historical variation of multiple biomarkers to determine a template, in accordance with some embodiments.

FIG. 11 shows a non-limiting example of a workflow of the platform described herein according to certain embodiments.

### DETAILED DESCRIPTION

Provided herein are computer-implemented platforms for real-time monitoring of biological of a subject, including results from point of need (PON) or point of care (POC) analyte detection systems (e.g., prognostic, diagnostic). The platforms described herein are configured to source biological data from an external source (e.g., electronic medical record), from an internal source (e.g., results from PON or PC device), or a user source (e.g., medical information). Storage of the health data employs, in some cases, encryption or similar strategies to ensure user privacy. In some cases, the platforms described herein are in communication with a sampling device, such as a POC device, that provides the results to the platform for real-time display to the user. The platforms described herein, in some embodiments, comprise a mobile processor that have a recommendation engine to provide recommendations to the user (e.g., the subject) based on, at least, the health data of the user. A communication engine in the platform communicates the real-time results and, in some cases, recommendations to the user via a user-friendly graphical user interface comprising elements that translate the complex health data into easy-to-understand terminology.

In a non-limiting example, of the computer-implemented platforms described herein, the sampling device can be configured to measure coagulation, relevant to a recommended dosing of anticoagulants (e.g., blood thinners). The sampling device measures repeatedly the coagulation values, which are stored as internally sourced data. The coagulation values are, in some cases, transmitted by the sampling device via a computer network, to the recommendation engine of the computer-implemented platform.

In addition, or in the alternative, the platform is configured to receive health data from a user source, including for example, nutrition information such as food types consumed, amount of alcohol intake or vitamin intake, other information such as illnesses, prescribed medications, prior existing conditions, planned medical procedures—all relevant to the metabolism of anticoagulant medications. Health data from the user source, in some embodiments, is transmitted to the recommendation engine.

Biological data from an external source, such as a genome-wide association study in a relevant population of patient, that reports statistically significant associations between certain genetic markers and a phenotype of interest (e.g., metabolism of anticoagulant medications), is transmitted to the recommendation engine of the platform in some cases. In some cases, a second sampling device configured to identify risk genotypes in the genotype of the subject is in communication via a computer network with the platform, and the polygenic risk score transmitted to the recommendation engine.

Biological data from one or more of the internal source, external source, or user source, is used to generate a recommendation templates. Recommendation templates of the instant disclosure include ranges and cutoffs for the coagulation value, which values are provided to the recommendation engine.

The platform enables users or healthcare professionals to modify the recommendation template using deep learning or other artificial or augmented intelligent algorithms to adjust cutoffs or ranges over time based on all available health data specific to that user. The recommendation engine then assesses the coagulation value in the context of all available health data and, based on the recommendation template, transmits the assessment to a recommendation generator to produce a recommendation.

Recommendations, in some embodiments, are stored in a template module and can include an automated request to: repeat testing for confirmation of results; contact a healthcare professional because values are out of range; to adjust the dosing based the assessed value; stop medication, or any combination thereof. In some embodiments, the recommendations are transmitted to an access control engine to apply permissions or otherwise restrict access to the recommendations and associated health data. In view of privacy considerations, the access control engine can be set to only allow sharing with a healthcare professional, service agent, family members, friends, care providers or any combination thereof.

### Computer-Implemented Platforms

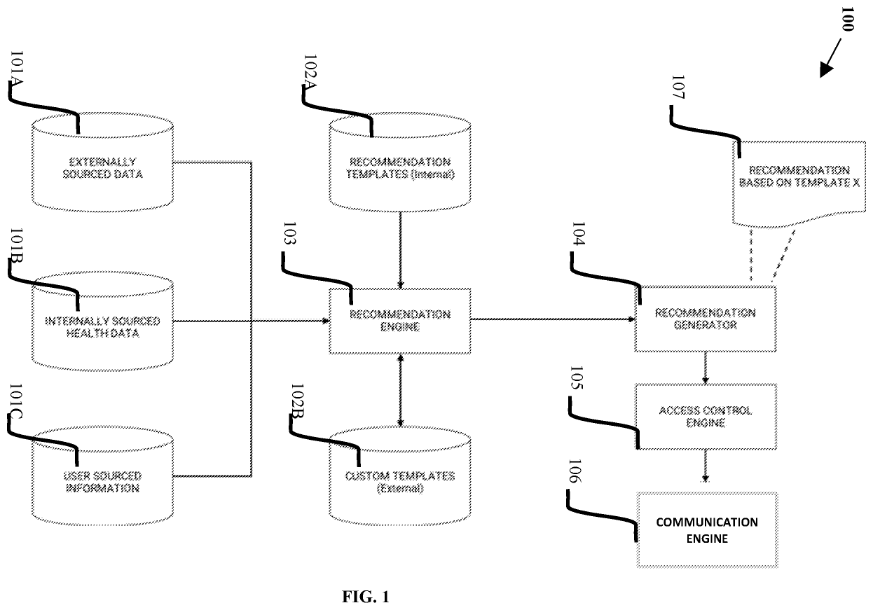

Disclosed herein, as shown in some embodiments in FIGS. 1-5E, is a computer-implemented platform for the real-time monitoring of health data of a subject. In some embodiments, the platform comprises a mobile processor configured to receive health data from a variety of sources, including from an internal source such as a sampling device described herein. In some embodiments, the sampling device is configured receive a biologic sample from a subject (as referred herein as “user”) and analyze the biologic sample to detect, for example, a quantity, a presence, or both of an analyte, relevant to the health of the subject.

In some embodiments, the user sourced information module can be aggregated into a population store. This aggregation can be performed in an anonymized fashion. Further, such aggregation can allow for population analysis and alert systems. Such population analysis can be performed by region, or other common features. Such alert systems can track disease outbreaks, map environmental impacts, or both.

FIG. 1 is a block diagram of an example computer-implemented recommendation platform 100. The platform can comprise: an externally sourced database 101A, an internally sourced biological database 101B, a user sourced information database 101C, a recommendation template database 102A, a recommendation engine 103, a custom template database 102B, a recommendation generator 104, an access control engine 105 and a communication engine 106.

In some embodiments, the externally sourced database 101A comprises a cost estimator, an electronic medical record, a prescription history, or any combination thereof. In some embodiments, the internally sourced biological database 101B comprises a presence of a biomarker. In some embodiments, the user sourced information database 101C comprises health data, medical information, lifestyle information, or any combination thereof. The health data can comprise a height, a weight, a heart rate, a blood pressure, or any combination thereof. The medical information can comprise a doctor name, a doctor contact information, a health insurance information, or any combination thereof. The lifestyle information can comprise food intake, exercise data, location, social information, or any combination thereof. The recommendation template database 102A can comprise service recommendation templates, product recommendation templates, health recommendation templates, social recommendation templates, or any combination thereof. The recommendation generator 104 can comprise a service recommendation, a product recommendation, a health recommendation, a social recommendation, or any combination thereof. The service recommendation can comprise a fitness coach, a nutritionist or any combination thereof. The product recommendation can comprise a therapeutic, a food, a drink, an exercise accessory, or any combination thereof. The social recommendation can comprise a support group.

Per FIG. 1, the data processor can be configured to provide a recommendation application, wherein the recommendation module comprises a reception module, a recommendation generation module, and a transmission module. The reception module can receive the user biological data and at least one of the quantity of the analyte or the presence of the analyte. The recommendation generation module can determine a recommendation based on the user biological data and at least one of the quantity of the analyte or the presence of the analyte. The transmission module can transmit the recommendation to the mobile processor.

The mobile processor can be configured to provide a mobile application, wherein the mobile application comprises a user sourced information module. The user sourced information module can receive a user biological data. In some embodiments, at least one of the mobile processor and the data processor are further configured to provide a sample module receiving the quantity of the analyte, the presence of the analyte, or both.

Different configurations of the elements herein can be used. The external data sources, the “sampling device” database, the user sourced information database, the recommendation template database, the recommendation engine, the custom template database, the recommendation generator, and the access control engine or any combination thereof, can be combined, further separated, distributed, or interchanged. The system can be implemented in a single device or distributed across multiple devices.

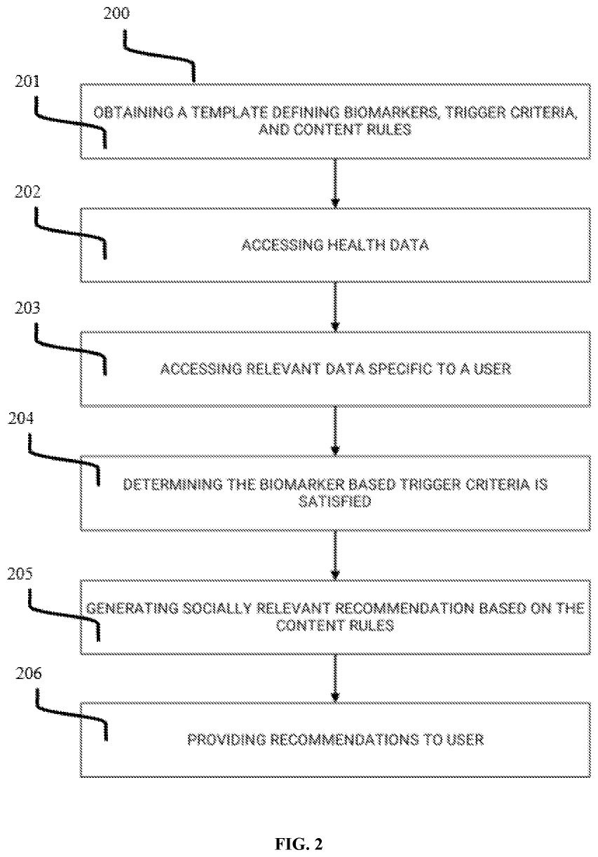

FIG. 2 shows an exemplary block diagram is a computer-implemented machine-learning based template generator 200 comprising: obtaining a template defining biomarkers, trigger criteria, and content rules 201; accessing biological data 202; accessing relevant data specific to a user 203; determining the biomarker based trigger criteria is satisfied 204; generating socially relevant recommendation based on the content rules 205; and providing recommendations to the user 206.

Also provided herein is a computer-implemented method comprising: receiving, by a sampling device, a biologic sample from the user; analyzing, by the sampling device, the biologic sample to detect a quantity, a presence, or both of an analyte; and receiving, by a mobile processor, a user biological data; receiving, by the mobile processor or a data processor, the quantity of the analyte, the presence of the analyte, or both; receiving, by the data processor, the user biological data and at least one of the quantity of the analyte or the presence of the analyte; generating, by the data processor, a recommendation based on the user biological data and at least one of the quantity of the analyte or the presence of the analyte; and transmitting the recommendation to the mobile processor

In some embodiments, the method further comprises receiving, by at least one of the user sourced information module or the reception module, an externally sourced data. In some embodiments, the externally sourced data comprises a website, a video, a document file, a medical record, a pharmacy record, a medication history, a health insurance information, a subscription information, or any combination thereof. In some embodiments the method further comprises storing, in a database a plurality of recommendation templates. In some embodiments the method further comprises selecting, by the data processor at least one recommendation template from the plurality of recommendation templates based on the user biological data and at least one of the quantity of the analyte or the presence of the analyte. In some embodiments the method further comprises determining, by the data processor, the recommendation based on the one or more selected recommendation templates. In some embodiments, the template comprises a trigger, a rule, or both. In some embodiments the method further comprises determining, by the data processor, the recommendation based on the trigger, the rule, or both. In some embodiments, the template is a pre-defined template or a custom template. In some embodiments, the template is determined by a machine-learning algorithm. In some embodiments the method further comprises confirming, by the data processor, an access of the recommendation to the user, a third party, or both. In some embodiments, transmitting, by the data processor, of the recommendation to the user, the one or more service agents, or both is based on the confirmation of access. In some embodiments the method further comprises transmitting, by the mobile processor, the recommendation to a service agent. A “service agent” as used herein may refer to a caregiver, healthcare professional, or any other service provider. In some instances, the service agent is a human. In some instances, the service agent is artificial intelligence (AI). In some embodiments, the transmission is based on the confirmation of access. In some embodiments, determining, by the data processor, the recommendation is performed by a machine learning algorithm. In some embodiments, the analyte comprises a predisposition biomarker, diagnostic biomarker, prognostic biomarker, predictive biomarker, DNA, RNA, protein, metabolite, circulating cell-free nucleic acid, or any combination thereof. In some embodiments, the user biological data comprises a weight, blood pressure, height, heart rate, food intake, nutritional history, activity history, sleep history, geolocation, body temperature, step count, body fat percentage, an emergency contact, a family contact, a friend contact, or any combination thereof. In some embodiments, the recommendation comprises a fitness recommendation, nutrition recommendation, mental health recommendation, a recommendation for further testing, or any combination thereof. In some embodiments, the recommendation for further testing relates to the sampling device, or additional testing device.

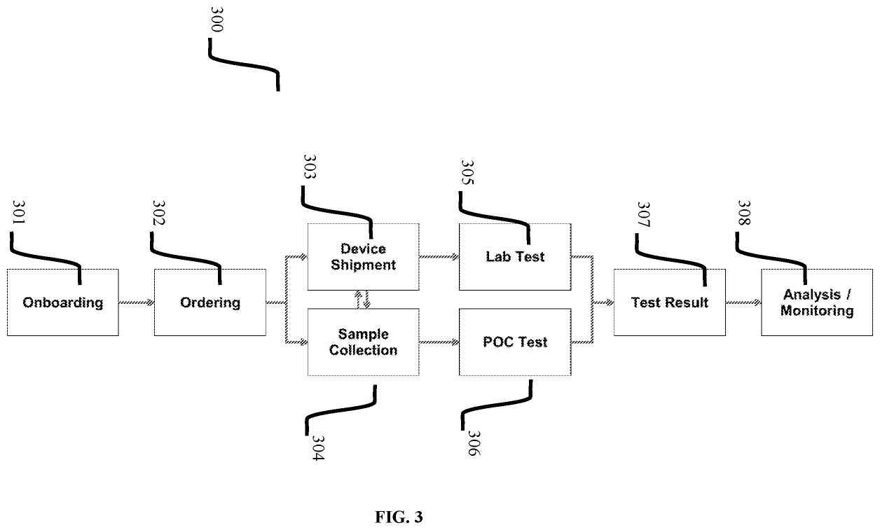

FIG. 3 shows another exemplary block diagram of a computer-implemented recommendation method 300. In some embodiments, the method comprises onboarding 301, ordering 302, device shipment 303, sample collection 304, a lab test 305, a POC test 306, a test result 307, and an analysis/monitoring 308.

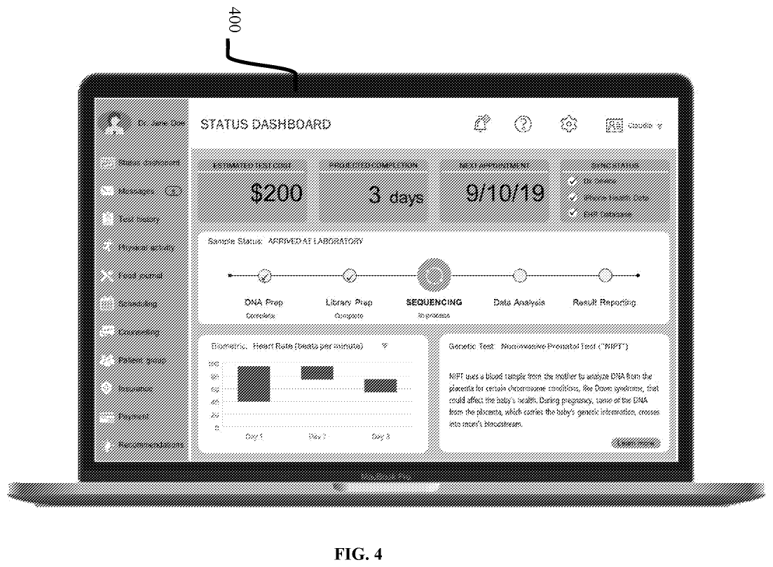

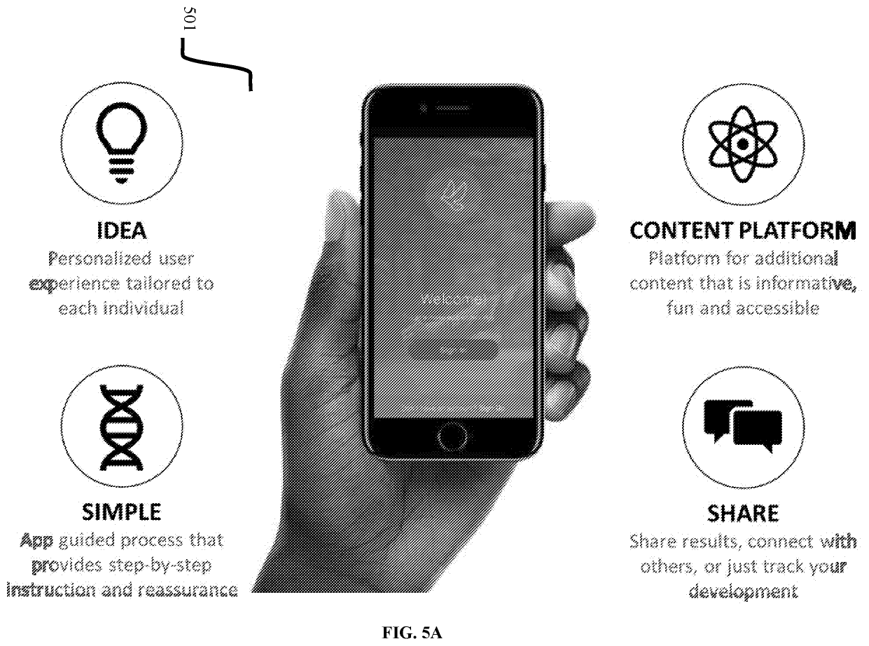

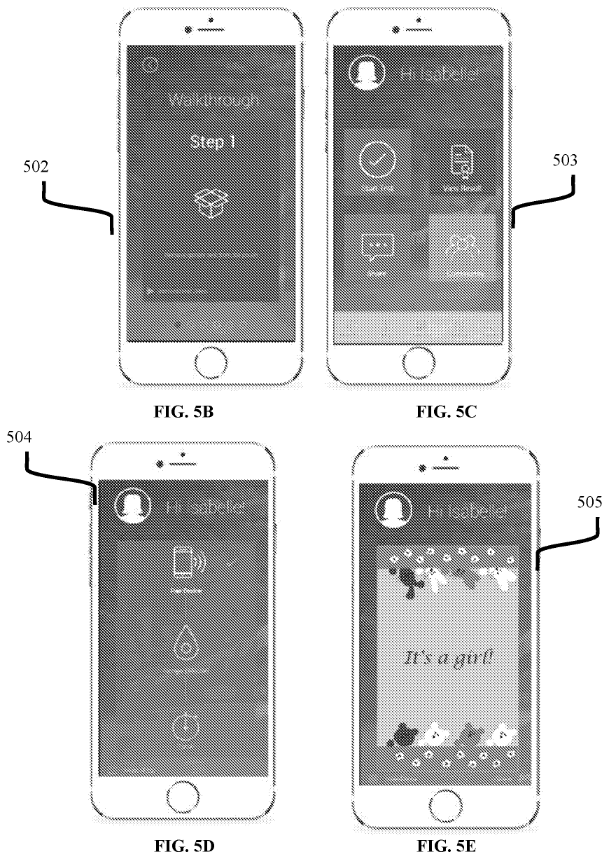

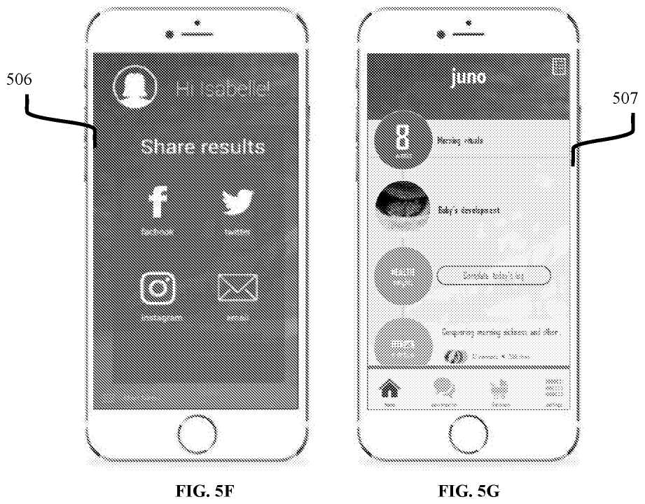

Exemplary generated user interfaces (GUIs) can be found in FIGS. 4-5E

FIG. 4 shows a first non-limiting example of a status dashboard of a mobile application. As seen the status dashboard can comprise values and charts regarding estimated cost, a projected completion, a next appointment date, a status of the Dx device, a status of the iPhone biological data, a status of the HER database, a heart rate. The dashboard can further comprise an up-to-date timeline of a sequencing test, and a genetic test description. The mobile application can further comprise a messenger, a test history database, a physical activity journal, a food journal, a scheduling module, a counseling module, a patient group module, an insurance module, a payment module, a recommendation module, or any combination thereof.

FIG. 5A shows a first non-limiting example of a mobile application configured to provide a personalized user experience tailored to each individual through an app guided process that provides step-by-step instruction and reassurance. Further, the application can act as a platform for additional content that is informative, fun, and accessible, and which allows users to share their results, connect with others, and track their development.

FIG. 5B shows a non-limiting example of a gender test on a mobile application, whereby in step 1 of a walkthrough, the user is instructed to “remove gender test from foil pouch,” and whereby an instructional video can be viewed for further instructions.

FIG. 5C shows a non-limiting example of a mobile application, whereby a start page greets a user and allows the user to start a test, view results, share results, access their community, or any combination thereof.

FIG. 5D shows a non-limiting example of a mobile application syncing with a sample device, and wherein a time is shown.

FIG. 5E shows a non-limiting example of a mobile application revealing the results of a gender test.

FIGS. 5F-G shows a non-limiting example of a mobile application sharing results through Facebook, twitter, Instagram, email, or any combination thereof.

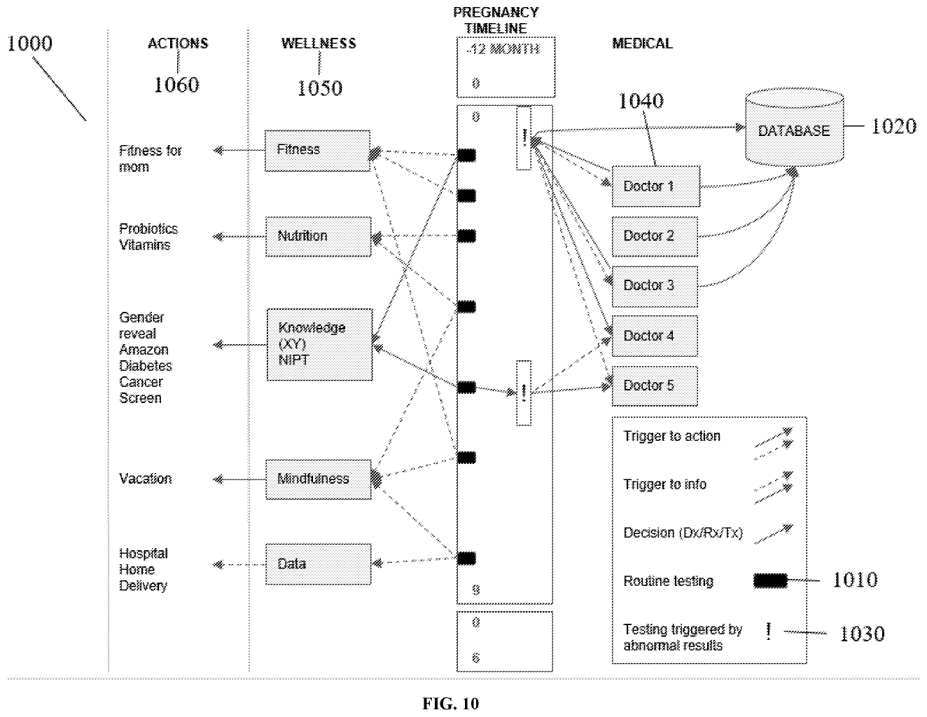

Referring FIG. 10, the computer-implemented platform 1000 integrates results from routine testing performed by a testing device 1010 (e.g., sampling device, or other external sampling device) and internally sourced data stored in an internally sourced database 1020, that in some cases, triggers testing triggered by abnormal results 1030. All results, including from the testing triggered by abnormal results performed by a medical practitioner 1040, and results from a testing device (e.g., sampling device or other external device) are stored in the internally sourced database 1020. A decision regarding a diagnosis, prognosis or therapeutic intervention by the medical practitioner 1040 are also stored in the internally sourced database 1020.

In this example, the user is pregnant. Throughout the user's pregnancy, various routine testing is performed by the testing device 1010. Results from the routine testing performed by the testing device 1010 either alone, or in combination with biological data derived from a second database (externally sourced database), triggers a recommendation to be generated by the computer-implemented platform related to a wellness category 1050. A wellness category may include fitness, nutrition, knowledge, mindfulness, or health data. The biological data in this example, may include data reported in the published literature (e.g., genome wide association studies involving risk of pregnancy-related complications) or proprietary data obtained by consumer devices (e.g., activity trackers, genealogy tests, and the like). Recommended actions 1060 are generated based at least in part on the results from the testing device 1010 and/or the biological data. Recommended actions may include fitness recommendations, recommended probiotics or vitamins, prenatal testing outcomes (e.g., gender) or other diagnostic or prognostic outcomes (e.g., diabetes, cancer), recommended relaxation or vacation techniques, or recommended at home or hospital actions. In some embodiments, user data, such as genetic information, is correlated with information derived from wearables and/or with analyte generated through the device. For example, the user may have a predisposition for certain heart conditions, and would benefit from monitoring of heart rate/ECG information derived from a wearable as well as blood biomarkers such troponin levels. Furthermore, in case the genetic predisposition is not fully explored, the availability of user information from wearables and biomarkers could help to elucidate and validate the existence of a predisposition, which could then be used for health, wellness and medical recommendations. Aggregation of genetic information, other user information and biomarkers for the individual and a population can lead to discovery and validation of new diagnostic and therapeutic options.

### User Biological Data

Disclosed herein, in some embodiments, are user biological data that includes data relevant to the biological of a subject or user. Biological data, in some cases, is stored in a database described herein, such as an internally sourced biological database, an externally sourced database, or a user sourced information database. In some embodiments, the computer-implemented platforms and methods described herein analyze the biological data. In some embodiments, the biological data of a subject is analyzed by the platforms and methods described herein to provide a medical or lifestyle recommendation to the subject.

In some embodiments, the biological data are derived from an external source or a user source. In some cases, the external sources includes biological information that is not provided by the sampling device. For example, the external sources can include internet sources, e.g., websites, videos, documents, files, metabolic activity data, physical activity data, heart rate data, blood pressure data, metabolite data, sleep data, or other sources that are publicly available on the internet and/or wearable device. In another example, the external sources can include information sources with limited public availability. For example, the external sources can include the user's electronic medical records, pharmacy records, medication history, health insurance information, or subscription-based information sources. In some embodiments, the biological data comprises genetic data, genomic data, epigenetic information, microbiome information, proteomic and transcriptomic information, immune repertoire information, pharmacogenetics, or drug-drug interactions of a relevant population, such as in an genome-wide association study, or meta-analysis.

In some embodiments, the biological data is derived from an internally sourced biological database that comprises a data store for biomarker information that is identified by a sampling device. The biomarker, in some cases, is a predisposition biomarker, a diagnostic biomarker, a prognostic biomarker, or a predictive biomarker. The biomarker include proteins, nucleic acids, metabolites, carbohydrates or lipids, or combinations thereof. For example, the biomarker can be fetal circulating cell-free DNA used to screen for chromosomal abnormalities as well as the fetal sex of a pregnant mother. In another example, can be prostate-specific antigen (PSA) used to screen for prostate cancer. In some embodiments, the biological data comprises genetic data, genomic data, epigenetic information, microbiome information, proteomic and transcriptomic information, immune repertoire information, pharmacogenetics, or drug-drug interactions of the user.

Biomarker results can be produced in a longitudinal manner over weeks, months or years deposited in the internally sourced biological database. The continuous aggregation of biological data and longitudinal analysis of this biological data through, for example artificial intelligence systems, in some cases alerts the user or the user's healthcare professionals to changes in the user's health status. In some cases, the changes in the user's health status is translated by the platform into defined recommendations to the user. Biological information deposited in the externally coursed database or the user information database comprising genetic predispositions, dosing schedules, or both, can be additionally or alternatively analyzed by the platform.

In a non-limiting example, the platform measures longitudinally on a monthly basis a level of prostate-specific antigen (PSA) marker in a blood sample obtained from a subject. A rise in PSA in the blood over time triggers an alert to a healthcare professional of the change in biomarker level. In some cases, the platform generates a recommendation to the subject to contact the health professional for follow-up diagnostic testing or prophylaxis. In some embodiments, longitudinal measurement of biomarkers associated response to various treatments is performed to determine a probability of therapeutic response to a given therapy.

In another non-limiting example, glucose, Hemoglobin A1C (HbA1c), or both, are biomarkers that are measured repeatedly in a blood sample obtained from a subject. A change in glucose and/or HbA1c in the blood over time generates a recommended insulin dosage to the subject, or triggers an alert to a healthcare professional of the change in biomarker level.

Circulating cell-free nucleic acids, metabolites, proteins, or any combination thereof can be analyzed longitudinally to monitor disease onset and progression. Such diseases can include but are not limited to, autoimmune diseases, cancer, heart disease, Neurological diseases such as Alzheimer and Parkinson disease, and multiple sclerosis. The analysis may include monitoring epigenetic markers specific to oligodendrocytes to monitor for flare ups. Increases in the amount of measured indicated flare-ups could cause the platform to recommend seeing a healthcare professional and/or adjust their recommended medication.

The user sourced information database can be a data store that stores user information that is specific to a user. User sourced information that is specific to a user can include information not generated by the sampling device. For example, the user sourced information is can include biological data, e.g., weight, height, blood pressure, heart rate, or other personal health data. In some cases, the personal health data includes vitals, weight, blood pressure, heart rate, body composition, body mass index (BMI), microbiome data, body fat percentage, oxygen output, lung capacity, or any combination thereof. In some cases, the personal health data is collected through wearable devices worn by the subject, or the sampling device.

In another example, the user sourced information can include medical information, e.g., current healthcare provider, health insurance information, medication history, or other medical information. In another example, the user sourced information can include lifestyle data, e.g., food intake/nutritional history, exercise/activity history, sleep history, geolocation, or other lifestyle data.

In another example, the user sourced information can include other information, e.g., contact information for friends and family. In some cases, other information includes emergency contact information, medical health care provider, primary care physician or specialist, whether the subject is a designated organ donor, blood type, known allergies, and the like.

### Recommendation Templates

The recommendation template database can be a data store that stores recommendation templates. The recommendation templates can be templates that define trigger criteria for presenting recommendations or notifications and define content rules for determining content to include in the recommendations or notifications. For example, a recommendation template can define to present a notification to a user if the “sampling device detects the presence of a biomarker above or below a specified range.

The recommendation templates can be pre-defined templates that can be shared for users. For example, the templates stored in the recommendation template database can be templates that can be applicable to all users. In another example, the templates stored in the recommendation template database can comprise templates that are pre-defined to be applicable to a subset of users.

The custom template database can be stored in a database. Custom templates can be recommendation templates that are customized for a particular user or generated from machine-based learning. For example, the custom template database can include recommendation templates defined by a particular user for use by one or more users, and not use by other users. In another example, the custom template database can include recommendation templates that are generated by a machine-learning based template generator for one or more users.

FIG. 9 shows a non-limiting example of template paths. The template paths can comprise a service path. The service path may be a medical path 901, a social recommendation path 902, an educational path 903, a professional path 904, or any combination thereof. The service path may further comprise a wellness path. In such embodiments, the sampling device can be connected to service actor that determines an appropriate template path for a particular user context. In some implementations, the user is directed to a path by a service actor.

The alternative service paths can be based on biological data representing multiple entities within the same physical body. As a result, the platform enables a service actors to inform and make recommendations based on such data. The platform also enables a feedback mechanism to select service actors based on the results received through objective biological data analysis augmented by other sources. For example improved service actors enable better particular biological, social, educational, commercial, outcomes.

### Rules and Triggers

In some implementations, the content rules can specify possible actions and recommendations that can be included in a notification. For example, the content rules can specify a recommendation for a local doctor if the user does not currently have a care physician. In another example, the content rules can specify that a notification that documents are available regarding an upcoming topic to be discussion during a doctor's visit.

The recommendation engine can receive information from one or more of the external sources, the sampling device database, or the user sourced information database, receive recommendation templates from one or more of the recommendation template database or the custom templates, and determine when to trigger presenting a recommendation or notification.

The recommendation engine can determine when to trigger generation of a notification based on the recommendation templates and information from one or more of the external sources, the sampling device database, or the user sourced information database. For example, the recommendation engine can determine when to trigger generation of a notification indicating that the fetal sex of a baby is male based on the presence of Y-Chromosome in fetal circulating cell-free DNA. In a more particular example, the recommendation engine can apply trigger criteria of a recommendation template and determine that the user cannot be receiving the recommended amounts of micronutrients from the user's diet, and in response recommend prenatal vitamins.

In some implementations, the recommendation engine can determine whether trigger criteria is satisfied when a change is detected in the external sources, the sampling device, or user sourced information. For example, the recommendation engine can determine that a particular source of information has changed, in response, identify recommendation templates with which trigger criteria can now be satisfied, and in response, can determine whether trigger criteria is satisfied for those identified recommendation templates. In another example, the recommendation engine can determine a threshold amount of data specified by an recommendation template has been gathered or analyzed, and in response, can determine whether trigger criteria is satisfied for the recommendation template. In some implementations, the recommendation engine can determine the change to detect based on information indicated by the recommendation template. For example, the recommendation template can define determining whether trigger criteria is satisfied when a particular source of information has changed. In another example, the recommendation template can define determining whether trigger criteria is satisfied when a threshold amount of data specified by a recommendation template has been gathered or analyzed.

In some implementations, the recommendation engine can determine whether trigger criteria is satisfied in response to an events or inputs from a device of a user. For example, a device of the user can supply health data, e.g., blood pressure, weight, heart rate, or other health data, and in response, prompt the recommendation engine to determine whether trigger criteria for one or more recommendation templates associated with recommendations or notifications associated with fitness, nutrition, or general well-being.

The prompt can additionally indicate to the recommendation engine to obtain updated information from one or more of the external sources, the sampling device database, or the user sourced information database. For example, a prompt can indicate to the recommendation engine to obtain updated insurance information to estimate potential cost of medical services. In another example, a prompt can indicate to the recommendation engine to obtain an updated medication history for a user from the user sourced information database.

### Access Control Engine

The access control engine can determine whether a user that would receive the recommendation or notification is permitted to receive the contents of the notification. For example, the access control engine can determine whether a user that would receive a notification indicating when other users attending a meeting are expected to be in attendance has permissions to receive information regarding the other user's biological data. In another example, the access control engine can determine whether a user that would receive a notification indicating the result of a laboratory test.

The access control engine can determine whether a user that would receive a notification is permitted to receive the contents of the notification based on access control information associated with content, where the access control information indicates what users or groups of users can receive the content. For example, the access control engine can determine if a user, e.g., health professional, family, or other user that would receive a notification including the results of a laboratory test, is identified by access control information for the health data of the user.

If the access control engine determines that the user that would receive the notification does not have permission to receive the contents of the notification, the access control engine can prevent the content from being presented to the user. For example, the access control engine may block the notification from being presented to the user. In another example, the access control engine can block the notification generator from receiving an indication to generate a notification from the recommendation engine.

In some implementations, the access control engine can modify a recommendation or notification based on a definition in a recommendation template for the notification. For example, the recommendation template can define that if the access control engine determines that a user is not permitted to receive information indicating the results of a particular lab test, the access control engine should provide the user a notification indicating that the test has been completed by the laboratory and trigger a notification to the ordering physician.

In some implementations, the access control engine can manage providing notifications based on actions permitted by the user. For example, the access control engine can determine that a user is permitted to share a notification that includes particular content, and in response to determining that the user is permitted to share a notification that includes particular content, provide a notification that can be shared. In another example, the access control engine can determine that a user is not permitted to share a notification that includes particular content, and in response to determining that the user is not permitted to share a notification that includes particular content, provide a notification that cannot be shared by the user.

### Computing System

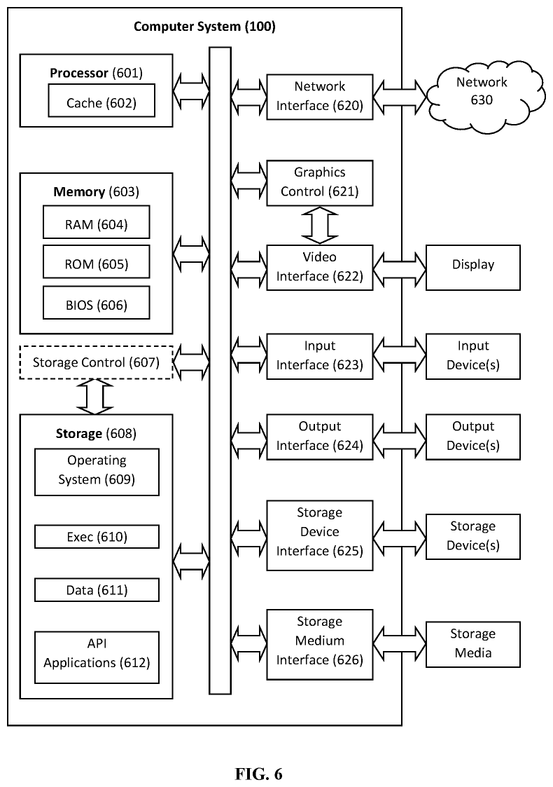

Referring to FIG. 6, a block diagram is shown depicting an exemplary machine that includes a computer system 600 (e.g., a processing or computing system) within which a set of instructions can execute for causing a device to perform or execute any one or more of the aspects and/or methodologies for static code scheduling of the present disclosure. The components in FIG. 6 are examples only and do not limit the scope of use or functionality of any hardware, software, embedded logic component, or a combination of two or more such components implementing particular embodiments.

Computer system 600 can include one or more processors 601, a memory 603, and a storage 608 that communicate with each other, and with other components, via a bus 640. The bus 640 can also link a display 632, one or more input devices 633 (which may, for example, include a keypad, a keyboard, a mouse, a stylus, etc.), one or more output devices 634, one or more storage devices 635, and various tangible storage media 636. All of these elements can interface directly or via one or more interfaces or adaptors to the bus 640. For instance, the various tangible storage media 636 can interface with the bus 640 via storage medium interface 626. Computer system 600 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 600 includes one or more processor(s) 601 (e.g., central processing units (CPUs) or general purpose graphics processing units (GPGPUs)) that carry out functions. Processor(s) 601 optionally contains a cache memory unit 602 for temporary local storage of instructions, data, or computer addresses. Processor(s) 601 are configured to assist in execution of computer readable instructions. Computer system 600 can provide functionality for the components depicted in FIG. 6 as a result of the processor(s) 601 executing non-transitory, processor-executable instructions embodied in one or more tangible computer-readable storage media, such as memory 603, storage 608, storage devices 635, and/or storage medium 636. The computer-readable media can store software that implements particular embodiments, and processor(s) 601 can execute the software. Memory 603 can read the software from one or more other computer-readable media (such as mass storage device(s) 635, 636) or from one or more other sources through a suitable interface, such as network interface 620. The software can cause processor(s) 601 to carry out one or more processes or one or more steps of one or more processes described or illustrated herein. Carrying out such processes or steps can include defining data structures stored in memory 603 and modifying the data structures as directed by the software.

The memory 603 can include various components (e.g., machine readable media) including, but not limited to, a random access memory component (e.g., RAM 604) (e.g., static RAM (SRAM), dynamic RAM (DRAM), ferroelectric random access memory (FRAM), phase-change random access memory (PRAM), etc.), a read-only memory component (e.g., ROM 605), and any combinations thereof. ROM 605 can act to communicate data and instructions unidirectionally to processor(s) 601, and RAM 604 can act to communicate data and instructions bidirectionally with processor(s) 601. ROM 605 and RAM 604 can include any suitable tangible computer-readable media described below. In one example, a basic input/output system 606 (BIOS), including basic routines that help to transfer information between elements within computer system 600, such as during start-up, can be stored in the memory 603.

Fixed storage 608 is connected bidirectionally to processor(s) 601, optionally through storage control unit 607. Fixed storage 608 provides additional data storage capacity and can also include any suitable tangible computer-readable media described herein. Storage 608 can be used to store operating system 609, executable(s) 610, data 611, applications 612 (application programs), and the like. Storage 608 can also include an optical disk drive, a solid-state memory device (e.g., flash-based systems), or a combination of any of the above. Information in storage 608 may, in appropriate cases, be incorporated as virtual memory in memory 603.

In one example, storage device(s) 635 can be removably interfaced with computer system 600 (e.g., via an external port connector (not shown)) via a storage device interface 625. Particularly, storage device(s) 635 and an associated machine-readable medium can provide non-volatile and/or volatile storage of machine-readable instructions, data structures, program modules, and/or other data for the computer system 600. In one example, software can reside, completely or partially, within a machine-readable medium on storage device(s) 635. In another example, software can reside, completely or partially, within processor(s) 601.

Bus 640 connects a wide variety of subsystems. Herein, reference to a bus can encompass one or more digital signal lines serving a common function, where appropriate. Bus 640 can be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 600 can also include an input device 633. In one example, a user of computer system 600 can enter commands and/or other information into computer system 600 via input device(s) 633. Examples of an input device(s) 633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 633 can be interfaced to bus 640 via any of a variety of input interfaces 623 (e.g., input interface 623) including, but not limited to, serial, parallel, game port, USB, FIREWIRE, THUNDERBOLT, or any combination of the above.

In particular embodiments, when computer system 600 is connected to network 630, computer system 600 can communicate with other devices, specifically mobile devices and enterprise systems, distributed computing systems, cloud storage systems, cloud computing systems, and the like, connected to network 630. Communications to and from computer system 600 can be sent through network interface 620. For example, network interface 620 can receive incoming communications (such as requests or responses from other devices) in the form of one or more packets (such as Internet Protocol (IP) packets) from network 630, and computer system 600 can store the incoming communications in memory 603 for processing. Computer system 600 can similarly store outgoing communications (such as requests or responses to other devices) in the form of one or more packets in memory 603 and communicated to network 630 from network interface 620. Processor(s) 601 can access these communication packets stored in memory 603 for processing.

Examples of the network interface 620 include, but are not limited to, a network interface card, a modem, and any combination thereof. Examples of a network 630 or network segment 630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 630, can employ a wired and/or a wireless mode of communication. In general, any network topology can be used.

Information and data can be displayed through a display 632. Examples of a display 632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 632 can interface to the processor(s) 601, memory 603, and fixed storage 608, as well as other devices, such as input device(s) 633, via the bus 640. The display 632 is linked to the bus 640 via a video interface 622, and transport of data between the display 632 and the bus 640 can be controlled via the graphics control 621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 632, computer system 600 can include one or more other peripheral output devices 634 including, but not limited to, an audio speaker, a printer, a storage device, and any combinations thereof. Such peripheral output devices can be connected to the bus 640 via an output interface 624. Examples of an output interface 624 include, but are not limited to, a serial port, a parallel connection, a USB port, a FIREWIRE port, a THUNDERBOLT port, and any combinations thereof.

In addition or as an alternative, computer system 600 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4 ®, Microsoft® Xbox 360®, Microsoft Xbox One, Nintendo® Wii®, Nintendo® Wii U®, and Ouya®.

Disclosed herein are computing systems comprising a mobile processor and a data processor. In some embodiments, a sampling device is in communication with the mobile processor and the data processor via a computer network. In some embodiments, the computer network is a wireless computer network. In some embodiments, the sampling device measures the presence, absence, or level of a biomarker in a sample obtained from the subject, as described herein.

In some cases, the mobile processor is configured to provide a mobile application comprising one or more of a user sourced information module receiving a user biological data. In some cases, the mobile processor is configured to provide a mobile application comprising one or more of a externally sourced information module receiving a user biological data. In some cases, the mobile processor is configured to provide a mobile application comprising one or more of a internally sourced information module receiving a user biological data. In some cases, the mobile application comprises a sample module that is configured to receive the quantity of the analyte, the presence of the analyte, or both, from the sampling device. In some cases, the mobile application comprises a reception module receiving the user biological data and at least one of the quantity of the analyte or the presence of the analyte from the sample module.

The externally sourced information module, in some cases, receives biological data from websites, videos, files, documents, or devices (external or internal). Externally sourced data are selected from metabolic activity data, physical activity data, heart rate data, blood pressure data, metabolite data, sleep data, electronic medical records, pharmacy records, medication history, health insurance information, or subscription-based information sources. The user sourced information module, in some cases, receives biological data from the user that is not generally available to the public or otherwise externally sourced.

The internally sourced information module, in some cases, receives the quantity of the analyte, the presence of the analyte, or both from the sampling device. In some cases, the biomarker includes a level, a presence or an absence of proteins, nucleic acids, metabolites, carbohydrates or lipids, or combinations thereof (e.g., fetal circulating cell-free DNA used to screen for chromosomal abnormalities as well as the fetal sex of a pregnant mother, levels of prostate-specific antigen (PSA) used to screen for prostate cancer).

The sample module, in some cases, is configured to receive the quantity of the analyte, the presence of the analyte, or both, from the sampling device. The reception module, in some cases, is configured to receive the user biological data and at least one of the quantity of the analyte or the presence of the analyte from the sample module

In some cases, the data processor is configured to provide a recommendation application, to generate a recommendation to the subject based on biological data received from one or more of the internally sourced biological database, externally sourced database, and user sourced information database. In some cases, the recommendation application comprises one or more of: (a) a reception module receiving the user biological data and at least one of the quantity of the analyte or the presence of the analyte; (b) a recommendation generation module determining a recommendation based on the user biological data and at least one of the quantity of the analyte or the presence of the analyte; and (c) a transmission module transmitting the recommendation to the mobile processor.

In some cases, at least one of the mobile processor and the data processor are further configured to provide a sample module receiving the quantity of the analyte, the presence of the analyte, or both. In some cases, the recommendation application comprises an access control module for confirming access of the recommendation to the user, a third party, or both. In some cases, the recommendation application comprises user sourced information module, an externally sourced information module, or an internally sourced information module. In some cases, the recommendation application comprises a template selection module.

The reception module, in some cases, is configured to receive the user biological data and at least one of the quantity of the analyte or the presence of the analyte from the sample module. The recommendation generation module, in some cases, is configured to generate a recommendation to the subject based on one or more of a recommendation template, and biological data for the subject. The transmission module, in some cases, transmits the recommendation to the user, the one or more service agents, or both based on the confirmation of access. The access control module, in some cases, confirms an access of the recommendation to the user, a third party, or both. The template selection module, in some cases, selects at least one recommendation template from the plurality of recommendation templates based on the user biological data and at least one of the quantity of the analyte or the presence of the analyte.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the computer-implemented platforms disclosed herein include at least one computer program, or use of the same. A computer program includes a sequence of instructions, executable by one or more processor(s) of the computing device's CPU, written to perform a specified task. Computer readable instructions can be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program can be written in various versions of various languages.

The functionality of the computer readable instructions can be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application can be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

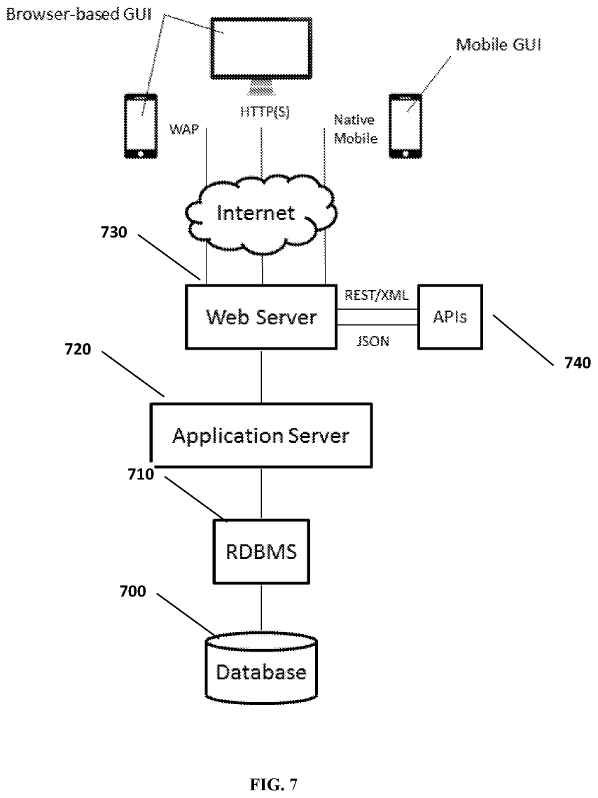

Referring to FIG. 7, in a particular embodiment, an application provision system comprises one or more databases 700 accessed by a relational database management system (RDBMS) 710. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 720 (such as Java servers, .NET servers, PHP servers, and the like) and one or more web servers 730 (such as Apache, IIS, GWS and the like). The web server(s) optionally expose one or more web services via app application programming interfaces (APIs) 740. Via a network, such as the Internet, the system provides browser-based and/or mobile native user interfaces.

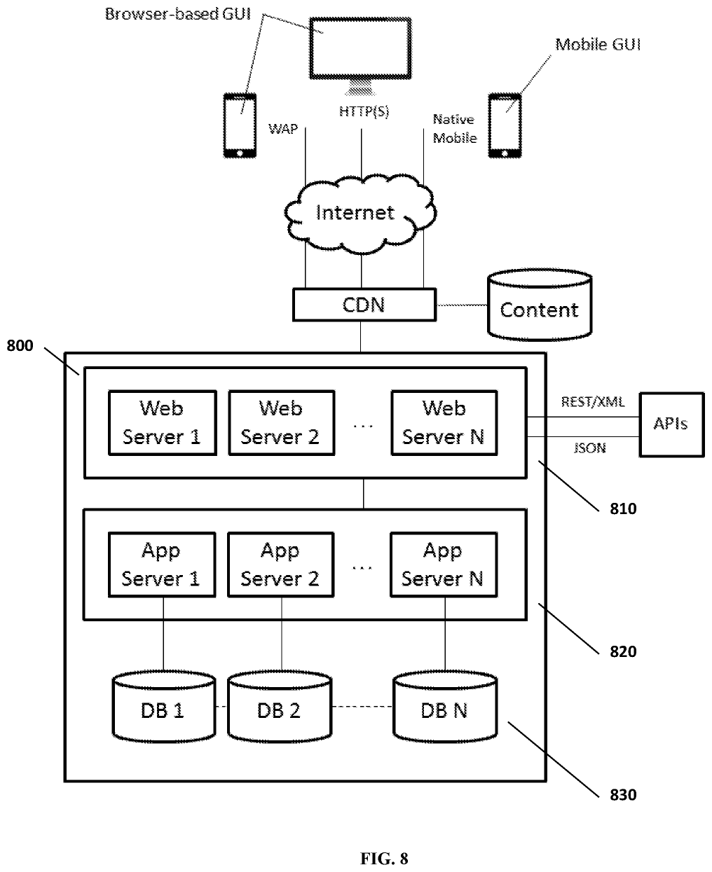

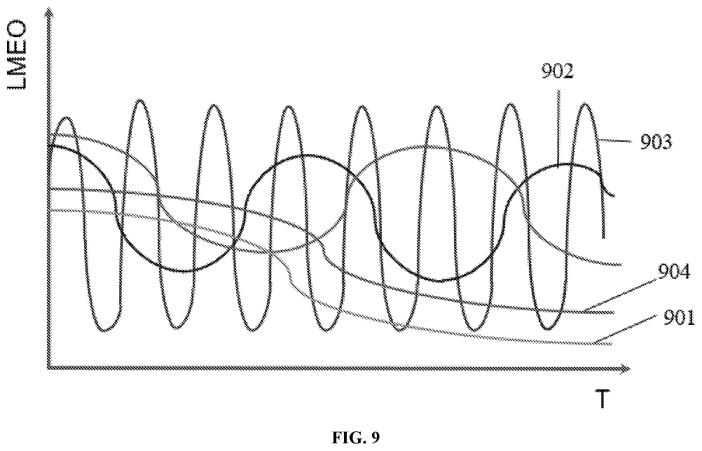

Referring to FIG. 8, in a particular embodiment, an application provision system alternatively has a distributed, cloud-based architecture 800 and comprises elastically load balanced, auto-scaling web server resources 810 and application server resources 820 as well synchronously replicated databases 830.

### Mobile Application

In some embodiments, sampling devices and systems disclosed herein comprise a digital processing device, or use of the same, wherein the digital processing device is provided with executable instructions in the form of a mobile application. In some embodiments, the mobile application is provided to a mobile digital processing device at the time it is manufactured. In other embodiments, the mobile application is provided to a mobile digital processing device via the computer network described herein. Mobile applications disclosed herein can be configured to locate, encrypt, index, and/or access information. Mobile applications disclosed herein can be configured to acquire, encrypt, create, manipulate, index, and peruse data.

Referring to FIG. 5A, in a particular embodiment, a mobile application is configured to connect with, communicate with, and receive biological data and other information from the sampling devices and systems disclosed herein. FIG. 5A is a diagram depicting various functions that the mobile application optionally provides to users. In this embodiment, the mobile application optionally provides: 1) a personalized, tailored user experience (UX) based on the personal information and preferences of the user; 2) an interactive text-, audio-, and/or video-driven instructional experience to inform the user how to utilize the sampling devices and systems; 3) a content platform that provides the user with access to articles, news, media, games, and the like; and 4) tools for tracking and sharing information, test results, providing access to templates and/or service actors, and events.

Referring to FIG. 5B, in a particular embodiment, the mobile application optionally includes an interactive interface providing a step-by-step walkthrough to guide a user through use of the sampling devices and systems disclosed herein. In various embodiments, the interactive walkthrough includes text, images, animations, audio, video, and the like to inform and instruct the user.

Referring to FIG. 5C, in a particular embodiment, the mobile application optionally includes a home screen allowing a user to access the mobile application functionality disclosed herein. In this embodiment, the home screen includes a personalized greeting as well as interface elements allowing the user to start a test, view current and historic test results, share test results, and interact with a larger community of users.

Referring to FIG. 5D, in a particular embodiment, the mobile application optionally includes a progress diagram informing a user of the status of a process for connecting to a device, system, or kit disclosed herein. In this embodiment, the diagram shows all the steps and indicates the current step. The steps are: 1) pair with the device via, for example, Bluetooth; 2) detect a sample in the device; and 3) wait for the sample to be processed. In some embodiments, the diagram is interactive, animated, or augmented with media or other content.

Referring to FIG. 5E, in a particular embodiment, the mobile application optionally includes a social sharing screen allowing a user to access features to share test results. Many services, platforms, and networks are suitable for sharing test results and other information and events. Suitable social networking and sharing platforms include, by way of non-limiting examples, Facebook, YouTube, Twitter, LinkedIn, Pinterest, Google Plus+, Tumblr, Instagram, Reddit, VK, Snapchat, Flickr, Vine, Meetup, Ask.fm, Classmates, QQ, WeChat, Swarm by Foursquare, Kik, Yik Yak, Shots, Periscope, Medium, Soundcloud, Tinder, WhatsApp, Snap Chat, Slack, Musical.ly, Peach, Blab, Renren, Sina Weibo, Renren, Line, and Momo. In some embodiments, the test results are shared by SMS, MMS or instant message. In some embodiments, the test results are shared by email.

In some embodiments, the mobile application optionally includes a home screen allowing a user to access additional features such as a blog and timeline of important information and events related to the test results, which is optionally shared. In various embodiments, suitable information and events include those pertaining to clinical trial outcomes, newly marketed therapeutics, nutrition, exercise, fetal development, health, etc. In this embodiment, the home screen further includes access to user preferences and settings.

In some instances, sampling devices and systems disclosed herein are in communication with the mobile application. The mobile application can provide for obtaining a Patient ID and electronic health record (EHR), arranging device shipment (to and/or from a user), online ordering of test results. The mobile application can provide for tracking a device or a portion thereof (e.g., shipping/storage compartment), or information obtained with the device, from one point to another. Various points can be selected from shipping, home, sample processing laboratory, and physician's office.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, and Samsung® Apps.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

Disclosed herein, in some embodiments, are computer-implemented platforms comprises one or more processors configured with one or more software modules. In some cases, the one or more software modules are selected from the externally sourced information module, the user sourced information module, the internally sourced information module, the sample module, the reception module, the recommendation generation module, the transmission module, the access control module, and the template selection module.

In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, the externally sourced information module performs the step of receiving biological data from websites, videos, files, documents, or devices (external or internal). Externally sourced data are selected from metabolic activity data, physical activity data, heart rate data, blood pressure data, metabolite data, sleep data, electronic medical records, pharmacy records, medication history, health insurance information, or subscription-based information sources. In some embodiments, the user sourced information module performs the step of receiving biological data from the user that is not generally available to the public or otherwise externally sourced.

In some embodiments, the internally sourced information module performs the step of receiving the quantity of the analyte, the presence of the analyte, or both from the sampling device. In some cases, the biomarker includes a level, a presence or an absence of proteins, nucleic acids, metabolites, carbohydrates or lipids, or combinations thereof (e.g., fetal circulating cell-free DNA used to screen for chromosomal abnormalities as well as the fetal sex of a pregnant mother, levels of prostate-specific antigen (PSA) used to screen for prostate cancer).

In some embodiments, the sample module performs the step of receiving the quantity of the analyte, the presence of the analyte, or both, from the sampling device. In some embodiments, the reception module receives the user biological data and at least one of the quantity of the analyte or the presence of the analyte from the sample module.

In some embodiments, the recommendation generation module performs the step of generating a recommendation to the subject based on one or more of a recommendation template, and biological data for the subject. In some embodiments, the transmission module performs the step of transmitting the recommendation to the user, the one or more service agents, or both based on the confirmation of access. In some embodiments, the access control module confirms an access of the recommendation to the user, a third party, or both. In some embodiments, the template selection module selects at least one recommendation template from the plurality of recommendation templates based on the user biological data and at least one of the quantity of the analyte or the presence of the analyte.

### Databases

Disclosed herein, in some embodiments, are computer-implemented platforms comprises one or more databases selected from the user sourced information database, the externally sourced database and the internally sourced biological database. In view of the disclosure provided herein, those of skill in the art will recognize that many databases are suitable for storage and retrieval of recommendation information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Sampling Devices and Systems

In some aspects, disclosed herein are sampling devices and systems for obtaining genetic information from a biological sample. As described herein, sampling devices and systems disclosed herein allow a user to collect and test a biological sample at a location of choice to detect the presence and/or quantity of a target analyte in the sample. In some instances, sampling devices and systems disclosed herein are used in the foregoing methods. In some instances, sampling devices and systems disclosed herein comprise a sample purifier that removes at least one component (e.g., cell, cell fragment, protein) from a biological sample of a subject; a nucleic acid sequencer for sequencing at least one nucleic acid in the biological sample; and a nucleic acid sequence output for relaying sequence information to a user of the device, system or kit.

By way of non-limiting example, the user may be a pregnant subject and the region of interest may be a region on a Y chromosome. By way of non-limiting example, a region of interest may be in a gene implicated in a cancer, an autoimmune condition, a neurological disorder, a metabolic disorder, a cardiovascular disease, immunity (e.g., infection susceptibility or resistance), and drug metabolism. A gene implicated in a disease, disorder or condition is considered a gene that when mutated, deleted, copied, epigenetically modified, under- or overexpressed, changes at least one of a symptom, outcome, duration, or onset of the disease, disorder or condition.

In general, sampling devices and systems of the present disclosure, integrate multiple functions, e.g., purification, amplification, and detection of the target analyte (e.g., including amplification products thereof), and combinations thereof. In some instances, the multiple functions are carried out within a single assay assembly unit or a single device. In some instances, all of the functions occur outside of the single unit or device. In some instances, at least one of the functions occurs outside of the single unit or device. In some instances, only one of the functions occurs outside of the single unit or device. In some instances, the sample purifier, nucleic acid amplification reagent, oligonucleotide, and detection reagent or component are housed in a single device. In general, sampling devices and systems of the present disclosure comprise a display, a connection to a display, or a communication to a display for relaying information about the biological sample to one or more people.

In some instances, sampling devices and systems comprise an additional component disclosed herein. Non-limiting examples of an additional component include a sample transportation compartment, a sample storage compartment, a sample and/or reagent receptacle, a temperature indicator, an electronic port, a communication connection, a communication device, a sample collection device, and a housing unit. In some instances, the additional component is integrated with the device. In some instances, the additional component is not integrated with the device. In some instances, the additional component is housed with the sample purifier, nucleic acid amplification reagent, oligonucleotide, and detection reagent or component in a single device. In some instances, the additional component is not housed within the single device.

In some instances, sampling devices and systems disclosed herein comprise components to obtain a sample, extract cell-free nucleic acids, and purify cell-free nucleic acids. In some instances, sampling devices and systems disclosed herein comprise components to obtain a sample, extract cell-free nucleic acids, purify cell-free nucleic acids, and prepare a library of the cell-free nucleic acids. In some instances, sampling devices and systems disclosed herein comprise components to obtain a sample, extract cell-free nucleic acids, purify cell-free nucleic acids, and sequence cell-free nucleic acids. In some instances, sampling devices and systems disclosed herein comprise components to obtain a sample, extract cell-free nucleic acids, purify cell-free nucleic acids, prepare a library of the cell-free nucleic acids, and sequence the cell-free nucleic acids. By way of non-limiting example, components for obtaining a sample are a transdermal puncture device and a filter for obtaining plasma from blood. Also, by way of non-limiting example, components for extracting and purifying cell-free nucleic acids comprise buffers, beads and magnets. Buffers, beads and magnets can be supplied at volumes appropriate for receiving a general sample volume from a finger prick (e.g., 50-150 μl of blood).

In some instances, sampling devices and systems comprise a receptacle for receiving the biological sample. The receptacle can be configured to hold a volume of a biological sample between 1 μl and 1 ml. The receptacle can be configured to hold a volume of a biological sample between 1 μl and 500 μl. The receptacle can be configured to hold a volume of a biological sample between 1 μl and 200 μl. The receptacle can have a defined volume that is the same as a suitable volume of sample for processing and analysis by the rest of the device/system components. This would preclude the need for a user of the device, system or kit to measure out a specified volume of the sample. The user would only need to fill the receptacle and thereby be assured that the appropriate volume of sample had been delivered to the device/system. In some instances, sampling devices and systems do not comprise a receptacle for receiving the biological sample. In some instances, the sample purifier receives the biological sample directly. Similar to the description above for the receptacle, the sample purifier can have a defined volume that is suitable for processing and analysis by the rest of the device/system components. In general, sampling devices and systems disclosed herein are intended to be used entirely at point of care. However, in some instances, the user can want to preserve or send the analyzed sample to another location (e.g., lab, clinic) for additional analysis or confirmation of results obtained at point of care. By way of non-limiting example, the device/system can separate plasma from blood. The plasma can be analyzed at point of care and the cells from the blood shipped to another location for analysis. In some instances, sampling devices and systems comprise a transport compartment or storage compartment for these purposes. The transport compartment or storage compartment can be capable of containing a biological sample, a component thereof, or a portion thereof. The transport compartment or storage compartment can be capable of containing the biological sample, portion thereof, or component thereof, during transit to a site remote to the immediate user. The transport compartment or storage compartment can be capable of containing cells that are removed from a biological sample, so that the cells can be sent to a site remote to the immediate user for testing. Non-limiting examples of a site remote to the immediate user can be a laboratory or a clinic when the immediate user is at home. In some instances, the home does not have a machine or additional device to perform an additional analysis of the biological sample. The transport compartment or storage compartment can be capable of containing a product of a reaction or process that result from adding the biological sample to the device. In some instances, the product of the reaction or process is a nucleic acid amplification product or a reverse transcription product. In some instances, the product of the reaction or process is a biological sample component bound to a binding moiety described herein. The biological sample component can comprise a nucleic acid, a cell fragment, an extracellular vesicle, a protein, a peptide, a sterol, a lipid, a vitamin, or glucose, any of which can be analyzed at a remote location to the user. In some instances, the transport compartment or storage compartment comprises an absorption pad, a paper, a glass container, a plastic container, a polymer matrix, a liquid solution, a gel, a preservative, or a combination thereof. An absorption pad or a paper can be useful for stabilizing and transporting a dried biological fluid with a protein or other biomarker for screening.

In some instances, sampling devices and systems disclosed herein provide for analysis of cell-free nucleic acids (e.g., circulating RNA and/or DNA) and non-nucleic acid components of a sample. Analysis of both cell-free nucleic acids and non-nucleic acid components can both occur at a point of need. In some instances, systems and devices provide an analysis of cell-free nucleic acids at a point of need and preservation of at least a portion or component of the sample for analysis of non-nucleic acid components at a site remote from the point of need. In some instances, systems and devices provide an analysis of non-nucleic acid components at a point of need and preservation of at least a portion or component of the sample for analysis of cell-free nucleic acids at a site remote from the point of need. These sampling devices and systems may be useful for carrier testing and detecting inherited diseases, such as those disclosed herein.

In some instances, the transport compartment or storage compartment comprises a preservative. The preservative can also be referred to herein as a stabilizer or biological stabilizer. In some instances, the device, system or kit comprises a preservative that reduces enzymatic activity during storage and/or transportation. In some instances, the preservative is a whole blood preservative. Non-limiting examples of whole blood preservatives, or components thereof, are glucose, adenine, citric acid, trisodium citrate, dextrose, sodium di-phosphate, and monobasic sodium phosphate. In some instances, the preservative comprises EDTA. EDTA can reduce enzymatic activity that would otherwise degrade nucleic acids. In some instances, the preservative comprises formaldehyde. In some instances, the preservative is a known derivative of formaldehyde. Formaldehyde, or a derivative thereof, can cross link proteins and therefore stabilize cells and prevent cell lysis.

In general, sampling devices and systems disclosed herein are intended to be used entirely at point of care. However, in some instances, the user may want to preserve or send the analyzed sample to another location (e.g., lab, clinic) for additional analysis or confirmation of results obtained at point of care. In some instances, sampling devices and systems comprise a transport compartment or storage compartment for these purposes. The transport compartment or storage compartment may be capable of containing a biological sample, a component thereof, or a portion thereof. The transport compartment or storage compartment may be capable of containing the biological sample, portion thereof, or component thereof, during transit to a site remote to the immediate user. Non-limiting examples of a site remote to the immediate user may be a laboratory or a clinic when the immediate user is at home. In some instances, the home does not have a machine or additional device to perform an additional analysis of the biological sample. The transport compartment or storage compartment may be capable of containing a product of a reaction or process that occurs in the device. In some instances, the product of the reaction or process is a nucleic acid amplification product or a reverse transcription product. In some instances, the product of the reaction or process is a biological sample component bound to a binding moiety described herein. The biological sample component may comprise a nucleic acid, cell fragment, an extracellular vesicle, a protein, a peptide, a sterol, a lipid, a vitamin, or glucose, any of which may be analyzed at a remote location to the user. In some instances, the transport compartment or storage compartment comprises an absorption pad, a paper, a glass container, a plastic container, a polymer matrix, a liquid solution, a gel, a preservative, or a combination thereof. In some instances, the device, system or kit comprises a stabilizer (chemical or structure (e.g., matrix)) that reduces enzymatic activity during storage and/or transportation.

Generally, sampling devices and systems disclosed herein are portable for a single person. In some instances, sampling devices and systems are handheld. In some instances, sampling devices and systems have a maximum length, maximum width or maximum height. In some instances, sampling devices and systems are housed in a single unit having a maximum length, maximum width or maximum height. In some instances the maximum length is not greater than 12 inches. In some instances the maximum length is not greater than 10 inches. In some instances the maximum length is not greater than 8 inches. In some instances the maximum length is not greater than 6 inches. In some instances the maximum width is not greater than 12 inches. In some instances the maximum width is not greater than 10 inches. In some instances the maximum width is not greater than 8 inches. In some instances the maximum width is not greater than 6 inches. In some instances the maximum width is not greater than 4 inches. In some instances the maximum height is not greater than 12 inches. In some instances the maximum height is not greater than 10 inches. In some instances the maximum height is not greater than 8 inches. In some instances the maximum height is not greater than 6 inches. In some instances the maximum height is not greater than 4 inches. In some instances the maximum height is not greater than 2 inches. In some instances the maximum height is not greater than 1 inch.

In some instances, sampling devices and systems disclosed herein comprise (a) a sample purifier that removes a cell from a biological fluid sample of a user subject; (b) at least one nucleic acid amplification reagent; (c) at least one oligonucleotide comprising a sequence corresponding to a region of interest, wherein the at least one oligonucleotide and nucleic acid amplification reagent are capable of producing an amplification product; and (d) at least one of a detection reagent or a signal detector for detecting the amplification product. In some instances, sampling devices and systems disclosed herein comprise a miniaturized digital nucleic acid amplification platform. By way of non-limiting example, the miniaturized nucleic acid amplification platform may be located on a chip within a device disclose herein, thereby keeping the entire device or system to a handheld size (e.g., similar to a cell phone). In some instances, the miniaturized nucleic acid amplification platform incorporates or is accompanied by digital output for ease of test result display.

In some instances, sampling devices and systems disclosed herein comprise (a) a sample purifier that removes a cell from a biological sample of a subject; (b) a nucleic acid sequencer for obtaining sequencing reads from nucleic acids in the biological sample; and (c) at least one of a detection reagent or a signal detector for detecting the sequencing reads. Non-limiting examples of a nucleic acid sequencer include next generation sequencing machines, nanopore sequencers, single molecule counters (e.g., counting sequences that are bar-coded/tagged).

### Sample Collection

In some instances, sampling devices and systems disclosed herein comprise a sample collector. In some instances, the sample collector is provided separately from the rest of the device, system or kit. In some instances, the sample collector is physically integrated with the device, system or kit, or a component thereof. In some instances, the sample collector is integrated with a receptacle described herein. In some instances, the sample collector can be a cup, tube, capillary, or well for applying the biological fluid. In some instances, the sample collector can be a cup for applying urine. In some instances, the sample collector can comprise a pipet for applying urine in the cup to the device, system or kit. In some instances, the sample collector can be a capillary integrated with a device disclosed herein for applying blood. In some instances, the sample collector can be tube, well, pad or paper integrated with a device disclosed herein for applying saliva. In some instances, the sample collector can be pad or paper for applying sweat.

In some instances, sampling devices and systems disclosed herein comprise a transdermal puncture device. Non-limiting examples of transdermal puncture devices are needles and lancets. In some instances, the sample collector comprises the transdermal puncture device. In some instances, sampling devices and systems disclosed herein comprise a microneedle, microneedle array or microneedle patch. In some instances, sampling devices and systems disclosed herein comprise a hollow microneedle. By way of non-limiting example, the transdermal puncture device is integrated with a well or capillary so that as the subject punctures their finger, blood is released into the well or capillary where it will be available to the system or device for analysis of its components. In some instances, the transdermal puncture device is a push button device with a needle or lancet in a concave surface. In some instances, the needle is a microneedle. In some instances, the transdermal puncture device comprises an array of microneedles. By pressing an actuator, button or location on the non-needle side of the concave surface, the needle punctures the skin of the subject in a more controlled manner than a lancet. Furthermore, the push button device can comprise a vacuum source or plunger to help draw blood from the puncture site.

### Sample Processing and Purification

Disclosed herein are sampling devices and systems that comprise a sample processor, wherein the sample processor modifies a biological sample to remove a component of the sample or separate the sample into multiple fractions (e.g., blood cell fraction and plasma or serum). The sample processor can comprise a sample purifier, wherein the sample purifier is configured to remove an unwanted substance or non-target component of a biological sample, thereby modifying the sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some instances, the sample purifier separates components of a biological sample disclosed herein. In some instances, sample purifiers disclosed herein remove components of a sample that would inhibit, interfere with or otherwise be detrimental to the later process steps such as nucleic acid amplification or detection. In some instances, the resulting modified sample is enriched for target analytes. This can be considered indirect enrichment of target analytes. Alternatively or additionally, target analytes can be captured directly, which is considered direct enrichment of target analytes.

In some instances, the sample purifier comprises a separation material for removing unwanted substances other than patient cells from the biological sample. Useful separation materials can include specific binding moieties that bind to or associate with the substance. Binding can be covalent or noncovalent. Any suitable binding moiety known in the art for removing a particular substance can be used. For example, antibodies and fragments thereof are commonly used for protein removal from samples. In some instances, a sample purifier disclosed herein comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof.

In some instances, sample purifiers disclosed herein comprise a filter. In some instances, sample purifiers disclosed herein comprise a membrane. Generally the filter or membrane is capable of separating or removing cells, cell particles, cell fragments, blood components other than cell-free nucleic acids, or a combination thereof, from the biological samples disclosed herein.

In some instances, the sample purifier facilitates separation of plasma or serum from cellular components of a blood sample. In some instances, the sample purifier facilitates separation of plasma or serum from cellular components of a blood sample before starting a molecular amplification reaction or a sequencing reaction. Plasma or serum separation can be achieved by several different methods such as centrifugation, sedimentation or filtration. In some instances, the sample purifier comprises a filter matrix for receiving whole blood, the filter matrix having a pore size that is prohibitive for cells to pass through, while plasma or serum can pass through the filter matrix uninhibited. In some instances, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that would contaminate target cell-free nucleic acids. Non-limiting examples of such filters include Pall Vivid™ GR membrane, Munktell Ahlstrom filter paper (see, e.g., WO2017017314), TeraPore filters.

In some instances sampling devices and systems disclosed herein employ vertical filtration, driven by capillary force to separate a component or fraction from a sample (e.g., plasma from blood). By way of non-limiting example, vertical filtration can comprise gravitation assisted plasma separation. A high-efficiency superhydrophobic plasma separator is described, e.g., by Liu et al., A High Efficiency Superhydrophobic Plasma Separation, Lab Chip 2015.

The sample purifier can comprise a lateral filter (e.g., sample does not move in a gravitational direction or the sample moves perpendicular to a gravitational direction). The sample purifier can comprise a vertical filter (e.g., sample moves in a gravitational direction). The sample purifier can comprise vertical filter and a lateral filter. The sample purifier can be configured to receive a sample or portion thereof with a vertical filter, followed by a lateral filter. The sample purifier can be configured to receive a sample or portion thereof with a lateral filter, followed by a vertical filter. In some instances, a vertical filter comprises a filter matrix. In some instances, the filter matrix of the vertical filter comprises a pore with a pore size that is prohibitive for cells to pass through, while plasma can pass the filter matrix uninhibited. In some instances, the filter matrix comprises a membrane that is especially suited for this application because it combines a large pore size at the top with a small pore size at the bottom of the filter, which leads to very gentle treatment of the cells preventing cell degradation during the filtration process.

In some instances, the sample purifier comprises an appropriate separation material, e.g., a filter or membrane, which removes unwanted substances from a biological sample without removing cell-free nucleic acids. In some instances, the separation material separates substances in the biological sample based on size, for example, the separation material has a pore size that excludes a cell but is permeable to cell-free nucleic acids. Therefore, when the biological sample is blood, the plasma or serum can move more rapidly than a blood cell through the separation material in the sample purifier, and the plasma or serum containing any cell-free nucleic acids permeates the holes of the separation material. In some instances, the biological sample is blood, and the cell that is slowed and/or trapped in the separation material is a red blood cell, a white blood cell, or a platelet. In some instances, the cell is from a tissue that contacted the biological sample in the body, including, but not limited to, a bladder or urinary tract epithelial cell (in urine), or a buccal cell (in saliva). In some instances, the cell is a bacterium or other microorganism.

In some instances, the sample purifier is capable of slowing and/or trapping a cell without damaging the cell, thereby avoiding the release of cell contents including cellular nucleic acids and other proteins or cell fragments that could interfere with subsequent evaluation of the cell-free nucleic acids. This can be accomplished, for example, by a gradual, progressive reduction in pore size along the path of a lateral flow strip or other suitable assay format, to allow gentle slowing of cell movement, and thereby minimize the force on the cell. In some instances, at least 95%, at least 98%, at least 99%, or up to 100% of the cells in a biological sample remain intact when trapped in the separation material. In addition to or independently of size separation, the separation material can trap or separate unwanted substances based on a cell property other than size, for example, the separation material can comprise a binding moiety that binds to a cell surface marker. In some instances, the binding moiety is an antibody or antigen binding antibody fragment. In some instances, the binding moiety is a ligand or receptor binding protein for a receptor on a blood cell or microvesicle.

In some instances, systems and devices disclosed herein comprise a separation material that moves, draws, pushes, or pulls the biological sample through the sample purifier, filter and/or membrane. In some instances, the material is a wicking material. Examples of appropriate separation materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable separation materials can be characterized as preventing passage of cells. In some instances, the separation material is not limited as long as it has a property that can prevent passage of the red blood cells. In some instances, the separation material is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials). In some instances, whole blood can be fractionated into red blood cells, white blood cells and serum components for further processing according to the methods of the present disclosure using a commercially available kit (e.g., Arrayit Blood Card Serum Isolation Kit, Cat. ABCS, Arrayit Corporation, Sunnyvale, Calif.).

In some instances the sample purifier comprises at least one filter or at least one membrane characterized by at least one pore size. In some instances, the sample purifier comprises multiple filters and/or membranes, wherein the pore size of at least a first filter or membrane differs from a second filter or membrane. In some instances, at least one pore size of at least one filter/membrane is about 0.05 microns to about 10 microns. In some instances, the pore size is about 0.05 microns to about 8 microns. In some instances, the pore size is about 0.05 microns to about 6 microns. In some instances, the pore size is about 0.05 microns to about 4 microns. In some instances, the pore size is about 0.05 microns to about 2 microns. In some instances, the pore size is about 0.05 microns to about 1 micron. In some instances, at least one pore size of at least one filter/membrane is about 0.1 microns to about 10 microns. In some instances, the pore size is about 0.1 microns to about 8 microns. In some instances, the pore size is about 0.1 microns to about 6 microns. In some instances, the pore size is about 0.1 microns to about 4 microns. In some instances, the pore size is about 0.1 microns to about 2 microns. In some instances, the pore size is about 0.1 microns to about 1 micron.

In some instances, the sample purifier is characterized as a gentle sample purifier. Gentle sample purifiers, such as those comprising a filter matrix, a vertical filter, a wicking material, or a membrane with pores that do not allow passage of cells, are particularly useful for analyzing cell-free nucleic acids. For example, prenatal applications of cell-free fetal nucleic acids in maternal blood are presented with the additional challenge of analyzing cell-free fetal nucleic acids in the presence of cell-free maternal nucleic acids, the latter of which create a large background signal to the former. By way of non-limiting example, a sample of maternal blood can contain about 500 to 750 genome equivalents of total cell-free DNA (maternal and fetal) per milliliter of whole blood when the sample is obtained without cell lysis or other cell disruption caused by the sample collection method. The fetal fraction in blood sampled from pregnant women can be around 10%, about 50 to 75 genome equivalents per ml. The process of obtaining cell-free nucleic acids usually involves obtaining plasma from the blood. If not performed carefully, maternal white blood cells can be destroyed, releasing additional cellular nucleic acids into the sample, creating a lot of background noise to the fetal cell-free nucleic acids. The typical white cell count is around 4*10\hat{ }6 to 10*10\hat{ }6 cells per ml of blood and therefore the available nuclear DNA is around 4,000 to 10,000 times higher than the overall cell-free DNA (cfDNA). Consequently, even if only a small fraction of maternal white blood cells is destroyed, releasing nuclear DNA into the plasma, the fetal fraction is reduced dramatically. For example, a white cell degradation of 0.01% can reduce the fetal fraction from 10% to about 5%. Sampling devices and systems disclosed herein aim to reduce these background signals.

In some instances, the sample processor is configured to separate blood cells from whole blood. In some instances, the sample processor is configured to isolate plasma from whole blood. In some instances, the sample processor is configured to isolate serum from whole blood. In some instances, the sample processor is configured to isolate plasma or serum from less than 1 milliliter of whole blood. In some instances, the sample processor is configured to isolate plasma or serum from less than 1 milliliter of whole blood. In some instances, the sample processor is configured to isolate plasma or serum from less than 500 μL of whole blood. In some instances, the sample processor is configured to isolate plasma or serum from less than 400 μL of whole blood. In some instances, the sample processor is configured to isolate plasma or serum from less than 300 μL of whole blood. In some instances, the sample processor is configured to isolate plasma or serum from less than 200 μL of whole blood. In some instances, the sample processor is configured to isolate plasma or serum from less than 150 μL of whole blood. In some instances, the sample processor is configured to isolate plasma or serum from less than 100 μL of whole blood.

In some instances, the biological sample comprises fetal trophoblasts, that in some cases, contain the genetic information of a fetus (e.g., RNA, DNA). In some instances, fetal trophoblasts are enriched in the biological sample, such as by using an antibody against a fetal cell-surface antigen of morphology (e.g., size, shape). In some instances, the fetal trophoblasts are (1) isolated from the biological sample; (2) the isolated trophoblasts are lysed; (3) the fetal nuclei from the lysed fetal trophoblasts are isolated; (4) lysing the isolated fetal nuclei; and (5) purifying the genomic DNA from the isolated fetal nuclei.

In some instances, sampling devices and systems disclosed herein comprise a binding moiety for producing a modified sample depleted of cells, cell fragments, nucleic acids or proteins that are unwanted or of no interest. In some instances, sampling devices and systems disclosed herein comprise a binding moiety for reducing cells, cell fragments, nucleic acids or proteins that are unwanted or of no interest, in a biological sample. In some instances, sampling devices and systems disclosed herein comprise a binding moiety for producing a modified sample enriched with target cell, target cell fragments, target nucleic acids or target proteins.

In some instances, sampling devices and systems disclosed herein comprise a binding moiety capable of binding a nucleic acid, a protein, a peptide, a cell surface marker, or microvesicle surface marker. In some instances, sampling devices and systems disclosed herein comprise a binding moiety for capturing an extracellular vesicle or extracellular microparticle in the biological sample. In some instances, the extracellular vesicle contains at least one of DNA and RNA. In some instances, sampling devices and systems disclosed herein comprise reagents or components for analyzing DNA or RNA contained in the extracellular vesicle. In some instances, the binding moiety comprises an antibody, antigen binding antibody fragment, a ligand, a receptor, a protein, a peptide, a small molecule, or a combination thereof.

In some instances, sampling devices and systems disclosed herein comprise a binding moiety capable of interacting with or capturing an extracellular vesicle that is released from a cell. In some instances, the cell is a fetal cell. In some instances, the cell is a placental cell. The fetal cell or the placental cell can be circulating in a biological fluid (e.g., blood) of a female pregnant subject. In some instances, the extracellular vesicle is released from an organ, gland or tissue. By way of non-limiting example, the organ, gland or tissue can be diseased, aging, infected, or growing. Non-limiting examples of organs, glands and tissues are brain, liver, heart, kidney, colon, pancreas, muscle, adipose, thyroid, prostate, breast tissue, and bone marrow.

By way of non-limiting example, sampling devices and systems disclosed herein can be capable of capturing and discarding an extracellular vesicle or extracellular microparticle from a maternal sample to enrich the sample for fetal/placental nucleic acids. In some instances, the extracellular vesicle is fetal/placental in origin. In some instances, the extracellular vesicle originates from a fetal cell. In some instances, the extracellular vesicle is released by a fetal cell. In some instances, the extracellular vesicle is released by a placental cell. The placental cell can be a trophoblast cell. In some instances, sampling devices and systems disclosed herein comprise a cell-binding moiety for capturing placenta educated platelets, which can contain fetal DNA or RNA fragments. These can be captured/enriched for with antibodies or other methods (low speed centrifugation). In such instances, the fetal DNA or RNA fragments can be analyzed as described herein to detect or indicate chromosomal information (e.g., gender). Alternatively or additionally, sampling devices and systems disclosed herein comprise a binding moiety for capturing an extracellular vesicle or extracellular microparticle in the biological sample that comes from a maternal cell.

In some instances, the binding moiety is attached to a solid support, wherein the solid support can be separated from the rest of the biological sample or the biological sample can be separated from the solid support, after the binding moiety has made contact with the biological sample. Non-limiting examples of solid supports include a bead, a nanoparticle, a magnetic particle, a chip, a microchip, a fibrous strip, a polymer strip, a membrane, a matrix, a column, a plate, or a combination thereof.

Sampling devices and systems disclosed herein can comprise a cell lysis reagent. Non-limiting examples of cell lysis reagents include detergents such as NP-40, sodium dodecyl sulfate, and salt solutions comprising ammonium, chloride, or potassium. Sampling devices and systems disclosed herein can have a cell lysis component. The cell lysis component can be structural or mechanical and capable of lysing a cell. By way of non-limiting example, the cell lysis component can shear the cells to release intracellular components such as nucleic acids. In some instances, sampling devices and systems disclosed herein do not comprise a cell lysis reagent. Some sampling devices and systems disclosed herein are intended to analyze cell-free nucleic acids.

### Nucleic Acid Amplification

Generally, sampling devices and systems disclosed herein are capable of amplifying a nucleic acid. Often sampling devices and systems disclosed herein comprise a DNA polymerase. In some instances, the sampling devices and systems disclosed herein comprise a reverse transcriptase enzyme to produce complementary DNA (cDNA) from RNA in biological samples disclosed herein, wherein the cDNA can be amplified and/or analyzed similarly to genomic DNA as described herein. Sampling devices and systems disclosed herein also often contain a crowding agent which can increase the efficiency enzymes like DNA polymerases and helicases. Crowding agents can increase an efficiency of a library, as described elsewhere herein. The crowding agent can comprise a polymer, a protein, a polysaccharide, or a combination thereof. Non-limiting examples of crowding agents that can be used in sampling devices and systems disclosed herein are dextran, poly(ethylene glycol) and dextran.

A traditional polymerase chain reaction requires thermocycling. This would be possible, but inconvenient for a typical at-home user without a thermocycler machine. In some instances, sampling devices and systems disclosed herein are capable of amplifying a nucleic acid without changing the temperature of the device or system or a component thereof. In some instances, sampling devices and systems disclosed herein are capable of amplifying a nucleic acid isothermally. Non-limiting examples of isothermal amplification are as follows: loop-mediated isothermal amplification (LAMP), strand displacement amplification (SDA), helicase dependent amplification (HDA), nicking enzyme amplification reaction (NEAR), and recombinase polymerase amplification (RPA). Thus, sampling devices and systems disclosed herein can comprise reagents necessary to carry out an isothermal amplification. Non-limiting examples of isothermal amplification reagents include recombinase polymerases, single-strand DNA-binding proteins, and strand-displacing polymerases. Generally, isothermal amplification using recombinase polymerase amplification (RPA) employs three core enzymes, recombinase, single-strand DNA-binding protein, and strand-displacing polymerase, to (1) pair oligonucleotide primers with homologous sequence in DNA, (2) stabilize displaced DNA strands to prevent primer displacement, and (3) extend the oligonucleotide primer using a strand displacing DNA polymerase. Using paired oligonucleotide primers, exponential DNA amplification can take place with incubation at room temperature (optimal at 37° C.).

In some instances, sampling devices and systems disclosed herein are capable of amplifying a nucleic acid at a temperature. In some instances, sampling devices and systems disclosed herein are capable of amplifying a nucleic acid at not more than two temperatures. In some instances, sampling devices and systems disclosed herein are capable of amplifying a nucleic acid at not more than three temperatures. In some instances, sampling devices and systems disclosed herein only require initially heating one reagent or component of the device, system or kit.

In some instances, sampling devices and systems disclosed herein are capable of amplifying a nucleic acid at a range of temperatures. In some instances, the range of temperatures is about −50° C. to about 100° C. In some instances, the range of temperatures is about −50° C. to about 90° C. In some instances, the range of temperatures is about −50° C. to about 80° C. In some instances, the range of temperatures is about is about −50° C. to about 70° C. In some instances, the range of temperatures is about −50° C. to about 60° C. In some instances, the range of temperatures is about −50° C. to about 50° C. In some instances, the range of temperatures is about −50° C. to about 40° C. In some instances, the range of temperatures is about −50° C. to about 30° C. In some instances, the range of temperatures is about −50° C. to about 20° C. In some instances, the range of temperatures is about −50° C. to about 10° C. In some instances, the range of temperatures is about 0° C. to about 100° C. In some instances, the range of temperatures is about 0° C. to about 90° C. In some instances, the range of temperatures is about 0° C. to about 80° C. In some instances, the range of temperatures is about is about 0° C. to about 70° C. In some instances, the range of temperatures is about 0° C. to about 60° C. In some instances, the range of temperatures is about 0° C. to about 50° C. In some instances, the range of temperatures is about 0° C. to about 40° C. In some instances, the range of temperatures is about 0° C. to about 30° C. In some instances, the range of temperatures is about 0° C. to about 20° C. In some instances, the range of temperatures is about 0° C. to about 10° C. In some instances, the range of temperatures is about 15° C. to about 100° C. In some instances, the range of temperatures is about 15° C. to about 90° C. In some instances, the range of temperatures is about 15° C. to about 80° C. In some instances, the range of temperatures is about is about 15° C. to about 70° C. In some instances, the range of temperatures is about 15° C. to about 60° C. In some instances, the range of temperatures is about 15° C. to about 50° C. In some instances, the range of temperatures is about 15° C. to about 40° C. In some instances, the range of temperatures is about 15° C. to about 30° C. In some instances, the range of temperatures is about 10° C. to about 30° C. In some instances, devices, systems, kits disclosed herein, including all components thereof, and all reagents thereof, are completely operable at room temperature, not requiring cooling, freezing or heating.

In some instances, at least a portion of the sampling devices and systems disclosed herein operate at about 20° C. to about 50° C. In some instances, at least a portion of the sampling devices and systems disclosed herein operate at about 37° C. In some instances, at least a portion of the sampling devices and systems disclosed herein operate at about 42° C. In some instances, the sampling devices and systems disclosed herein are advantageously operated at room temperature. In some instances, at least a portion of the sampling devices and systems disclosed herein are capable of amplifying a nucleic acid isothermally at about 20° C. to about 30° C. In some instances, at least a portion of the sampling devices and systems disclosed herein are capable of amplifying a nucleic acid isothermally at about 23° C. to about 27° C.

In some instances, sampling devices and systems disclosed herein comprise a hybridization probe with an a basic site, a fluorophore and quencher to monitor amplification. Exonuclease III can be included to cleave the basic site and release the quencher to allow fluorescent excitation. In some instances, amplification products are detected or monitored via lateral flow by attaching a capture molecule (e.g. Biotin) to one of the amplification primers and labeling a hybridization primer with a 5′-antigenic molecule (e.g. fluorescein derivative FAM) for capture to allow for detection. As such, in some instances, sampling devices and systems disclosed herein provide for detection of nucleic acids and amplification products on a lateral flow device. Lateral flow devices are described herein.

In some instances, sampling devices and systems disclosed herein comprise at least one nucleic acid amplification reagent and at least one oligonucleotide primer capable of amplifying a first sequence in a genome and a second sequence in a genome, wherein the first sequence and the second sequence are similar, and wherein the first sequence is physically distant enough from the second sequence such that the first sequence is present on a first cell-free nucleic acid of the subject and the second sequence is present on a second cell-free nucleic acid of the subject. In some instances, the at least two sequences are immediately adjacent. In some instances the at least two sequences are separated by at least one nucleotide. In some instances, the at least two sequences are separated by at least two nucleotides. In some instances, the at least two sequences are separated by at least about 5, at least about 10, at least about 15, at least about 20, at least about 30, at least about 40, at least about 50, or at least about 100 nucleotides. In some instances, the at least two sequences are at least about 50% identical. In some instances, the at least two sequences are at least about 60% identical, at least about 60% identical, at least about 60%, at least about 70%, at least about 80%, at least about 90%, at least about 95%, at least about 99%, or 100% identical. In some instances, the first sequence and the second sequence are each at least 10 nucleotides in length. In some instances, the first sequence and the second sequence are each at least about 10, at least about 15, at least about 20, at least about 30, at least about 50, or at least about 100 nucleotides in length. In some instances, the first sequence and the second sequence are on the same chromosome. In some instances, the first sequence is on a first chromosome and the second sequence is on a second chromosome. In some instances, the first sequence and the second sequence are in functional linkage. For example, all CpG sites in the promotor region of gene AOX1 show the same hypermethylation in prostate cancer, so these sites are in functional linkage because they functionally carry the same information but are located one or more nucleotides apart.

In some instances, sampling devices and systems disclosed herein comprise at least one of an oligonucleotide probe or oligonucleotide primer that is capable of annealing to a strand of a cell-free nucleic acid, wherein the cell-free nucleic acid comprises a sequence corresponding to a region of interest or a portion thereof. In some instances, the region of interest is a region of a Y chromosome. In some instances, the region of interest is a region of an X chromosome. In some instances, the region of interest is a region of an autosome. In some instances, the region of interest, or portion thereof, comprises a repeat sequence as described herein that is present in a genome more than once. In some instances, the region of interest is about 10 nucleotides to about 1,000,000 nucleotides in length. In some instances, the region of interest is at least 10 nucleotides in length. In some instances, the region of interest is at least 100 nucleotides in length. In some instances, the region is at least 1000 nucleotides in length. In some instances, the region of interest is about 10 nucleotides to about 500,000 nucleotides in length. In some instances, the region of interest is about 10 nucleotides to about 300,000 nucleotides in length. In some instances, the region of interest is about 100 nucleotides to about 1,000,000 nucleotides in length. In some instances, the region of interest is about 100 nucleotides to about 500,000 nucleotides in length. In some instances, the region of interest is about 100 nucleotides to about 300,000 base pairs in length. In some instances, the region of interest is about 1000 nucleotides to about 1,000,000 nucleotides in length. In some instances, the region of interest is about 1000 nucleotides to about 500,000 nucleotides in length. In some instances, the region of interest is about 1000 nucleotides to about 300,000 nucleotides in length. In some instances, the region of interest is about 10,000 nucleotides to about 1,000,000 nucleotides in length. In some instances, the region of interest is about 10,000 nucleotides to about 500,000 nucleotides in length. In some instances, the region of interest is about 10,000 nucleotides to about 300,000 nucleotides in length. In some instances, the region of interest is about 300,000 nucleotides in length.

In some instances, the sequence corresponding to the region of interest is at least about 5 nucleotides in length. In some instances, the sequence corresponding to the region of interest is at least about 8 nucleotides in length. In some instances, the sequence corresponding to the region of interest is at least about 10 nucleotides in length. In some instances, the sequence corresponding to the region of interest is at least about 15 nucleotides in length. In some instances, the sequence corresponding to the region of interest is at least about 20 nucleotides in length. In some instances, the sequence corresponding to the region of interest is at least about 50 nucleotides in length. In some instances, the sequence corresponding to the region of interest is at least about 100 nucleotides in length. In some instances, the sequence is about 5 nucleotides to about 1000 nucleotides in length. In some instances, the sequence is about 10 nucleotides to about 1000 nucleotides in length. In some instances, the sequence is about 10 nucleotides to about 500 nucleotides in length. In some instances, the sequence is about 10 nucleotides to about 400 nucleotides in length. In some instances, the sequence is about 10 nucleotides to about 300 nucleotides in length. In some instances, the sequence is about 50 nucleotides to about 1000 nucleotides in length. In some instances, the sequence is about 50 nucleotides to about 500 nucleotides in length.

In some instances, sampling devices and systems disclosed herein comprise at least one of an oligonucleotide probe and oligonucleotide primer that is capable of annealing to a strand of a cell-free nucleic acid, wherein the cell-free nucleic acid comprises a sequence corresponding to a sub-region of interest disclosed herein. In some instances, the sub-region is represented by a sequence that is present in the region of interest more than once. In some instances, the sub-region is about 10 to about 1000 nucleotides in length. In some instances, the sub-region is about 50 to about 500 nucleotides in length. In some instances, the sub-region is about 50 to about 250 nucleotides in length. In some instances, the sub-region is about 50 to about 150 nucleotides in length. In some instances, the sub-region is about 100 nucleotides in length.

In some instances, sampling devices and systems disclosed herein comprise at least one oligonucleotide primer, wherein the oligonucleotide primer has a sequence complementary to or corresponding to a Y chromosome sequence. In some instances, devices, systems and kits disclosed herein comprise a pair of oligonucleotide primers, wherein the pair of oligonucleotide primers have sequences complementary to or corresponding to a Y chromosome sequence. In some instances, devices, systems and kits disclosed herein comprise at least one oligonucleotide primer, wherein the oligonucleotide primer comprises a sequence complementary to or corresponding to a Y chromosome sequence. In some instances, devices, systems and kits disclosed herein comprise a pair of oligonucleotide primers, wherein the pair of oligonucleotide primers comprise sequences complementary to or corresponding to a Y chromosome sequence. In some instances, devices, systems and kits disclosed herein comprise at least one oligonucleotide primer, wherein the oligonucleotide primer consists of a sequence complementary to or corresponding to a Y chromosome sequence. In some instances, devices, systems and kits disclosed herein comprise a pair of oligonucleotide primers, wherein the pair of oligonucleotide primers consists of sequences complementary to or corresponding to a Y chromosome sequence. In some instances, the sequence(s) complementary to or corresponding to a Y chromosome sequence is at least 75% identical to a wild-type human Y chromosome sequence. In some instances, the sequence(s) complementary to or corresponding to a Y chromosome sequence is at least 80% identical to a wild-type human Y chromosome sequence. In some instances, the sequence(s) complementary to or corresponding to a Y chromosome sequence is at least 85% identical to a wild-type human Y chromosome sequence. In some instances, the sequence(s) complementary to or corresponding to a Y chromosome sequence is at least 80% identical to a wild-type human Y chromosome sequence. In some instances, the sequence(s) complementary to or corresponding to a Y chromosome sequence is at least 90% identical to a wild-type human Y chromosome sequence. In some instances, the sequence(s) complementary to or corresponding to a Y chromosome sequence is at least 95% identical to a wild-type human Y chromosome sequence. In some instances, the sequence(s) complementary to or corresponding to a Y chromosome sequence is at least 97% identical to a wild-type human Y chromosome sequence. In some instances, the sequence(s) complementary to or corresponding to a Y chromosome sequence is 100% identical to a wild-type human Y chromosome sequence.

In some instances, sampling devices and systems disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome region, or portion thereof, wherein the portion thereof has a given length. In some instances, the length of the portion thereof is about 10 nucleotides to about 100 nucleotides. In some instances, the length of the portion thereof is about 100 nucleotides to about 1000 nucleotides. In some instances, the length of the portion thereof is about 1000 nucleotides to about 10,000 nucleotides. In some instances, the length of the portion thereof is about 10,000 nucleotides to about 100,000 nucleotides.

In some instances, the region of interest is a Y chromosome region, or portion thereof, that comprises a sequence that is present on the Y chromosome more than once. In some instances, the Y chromosome region is located between position 20000000 and position 21000000 of the Y chromosome. In some instances, the Y chromosome region is located between position 20500000 and position 21000000 of the Y chromosome. In some instances, the Y chromosome region is located between position 20000000 and position 20500000 of the Y chromosome. In some instances, the Y chromosome region is located between position 20000000 and position 20250000 of the Y chromosome. In some instances, the Y chromosome region is located between position 20250000 and position 20500000 of the Y chromosome. In some instances, the Y chromosome region is located between position 20500000 and position 20750000 of the Y chromosome. In some instances, the Y chromosome region is located between position 20750000 and position 21000000 of the Y chromosome. In some instances, the Y chromosome region is located between position 20080000 and position 20400000 of the Y chromosome. In some instances, the Y chromosome region is located between position 20082000 and position 20351000 of the Y chromosome. In some instances, the Y chromosome region is located between position 20082183 and position 20350897 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region. In some instances, corresponding is 100% identical. In some instances, corresponding is at least 99% identical. In some instances, corresponding is at least 98% identical. In some instances, corresponding is at least 95% identical. In some instances, corresponding is at least 90% identical.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350799 and end position 20350897 of the Y chromosome. In some instances, the sequence corresponds to at least 10 nucleotides of a Y chromosome sub-region between start position 20350799 and end position 20350897 of the Y chromosome. In some instances, the sequence corresponds to at least 50 nucleotides of a Y chromosome sub-region between start position 20350799 and end position 20350897 of the Y chromosome. In some instances, the sequence corresponds to at least about 10 to at least about 1000 nucleotides of a Y chromosome sub-region between start position 20350799 and end position 20350897 of the Y chromosome. In some instances, the sequence corresponds to at least about 50 to at least about 500 nucleotides of a Y chromosome sub-region between start position 20350799 and end position 20350897 of the Y chromosome. In some instances, the sequence corresponds to at least about 50 to at least about 150 nucleotides of a Y chromosome sub-region between start position 20350799 and end position 20350897 of the Y chromosome.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20349236 and end position 20349318 of the Y chromosome. In some instances, the sequence corresponds to at least 10 nucleotides of a Y chromosome sub-region between start position 20349236 and end position 20349318 of the Y chromosome. In some instances, the sequence corresponds to at least 50 nucleotides of a Y chromosome sub-region between start position 20349236 and end position 20349318 of the Y chromosome. In some instances, the sequence corresponds to at least about 10 to at least about 1000 nucleotides of a Y chromosome sub-region between start position 20349236 and end position 20349318 of the Y chromosome. In some instances, the sequence corresponds to at least about 50 to at least about 500 nucleotides of a Y chromosome sub-region between start position 20349236 and end position 20349318 of the Y chromosome. In some instances, the sequence corresponds to at least about 50 to at least about 150 nucleotides of a Y chromosome sub-region between start position 20349236 and end position 20349318 of the Y chromosome.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350231 and end position 20350323 of the Y chromosome. In some instances, the sequence corresponds to at least 10 nucleotides of a Y chromosome sub-region between start position 20350231 and end position 20350323 of the Y chromosome. In some instances, the sequence corresponds to at least 50 nucleotides of a Y chromosome sub-region between start position 20350231 and end position 20350323 of the Y chromosome. In some instances, the sequence corresponds to at least about 10 to at least about 1000 nucleotides of a Y chromosome sub-region between start position 20350231 and end position 20350323 of the Y chromosome. In some instances, the sequence corresponds to at least about 50 to at least about 500 nucleotides of a Y chromosome sub-region between start position 20350231 and end position 20350323 of the Y chromosome. In some instances, the sequence corresponds to at least about 50 to at least about 150 nucleotides of a Y chromosome sub-region between start position 20350231 and end position 20350323 of the Y chromosome.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350601 and end position 20350699 of the Y chromosome. In some instances, the sequence corresponds to at least 10 nucleotides of a Y chromosome sub-region between start position 20350601 and end position 20350699 of the Y chromosome. In some instances, the sequence corresponds to at least 50 nucleotides of a Y chromosome sub-region between start position 20350601 and end position 20350699 of the Y chromosome. In some instances, the sequence corresponds to at least about 10 to at least about 1000 nucleotides of a Y chromosome sub-region between start position 20350601 and end position 20350699 of the Y chromosome. In some instances, the sequence corresponds to at least about 50 to at least about 500 nucleotides of a Y chromosome sub-region between start position 20350601 and end position 20350699 of the Y chromosome. In some instances, the sequence corresponds to at least about 50 to at least about 150 nucleotides of a Y chromosome sub-region between start position 20350601 and end position 20350699 of the Y chromosome.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20082183 and end position 20082281 of the Y chromosome. In some instances, the sequence corresponds to at least 10 nucleotides of a Y chromosome sub-region between start position 20082183 and end position 20082281 of the Y chromosome. In some instances, the sequence corresponds to at least 50 nucleotides of a Y chromosome sub-region between start position 20082183 and end position 20082281 of the Y chromosome. In some instances, the sequence corresponds to at least about 10 to at least about 1000 nucleotides of a Y chromosome sub-region between start position 20082183 and end position 20082281 of the Y chromosome. In some instances, the sequence corresponds to at least about 50 to at least about 500 nucleotides of a Y chromosome sub-region between start position 20082183 and end position 20082281 of the Y chromosome. In some instances, the sequence corresponds to at least about 50 to at least about 150 nucleotides of a Y chromosome sub-region between start position 20082183 and end position 20082281 of the Y chromosome.

In some instances, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 56673250 and end position 56771489 of the Y chromosome. In some instances, the sequence corresponds to at least 10 nucleotides of a Y chromosome sub-region between start position 56673250 and end position 56771489 of the Y chromosome. In some instances, the sequence corresponds to at least 50 nucleotides of a Y chromosome sub-region between start position 56673250 and end position 56771489 of the Y chromosome. In some instances, the sequence corresponds to at least about 10 to at least about 1000 nucleotides of a Y chromosome sub-region between start position 56673250 and end position 56771489 of the Y chromosome. In some instances, the sequence corresponds to at least about 50 to at least about 500 nucleotides of a Y chromosome sub-region between start position 56673250 and end position 56771489 of the Y chromosome. In some instances, the sequence corresponds to at least about 50 to at least about 150 nucleotides of a Y chromosome sub-region between start position 56673250 and end position 56771489 of the Y chromosome.

Any appropriate nucleic acid amplification method known in the art is contemplated for use in the devices and methods described herein. In some instances, isothermal amplification is used. In some instances, amplification is isothermal with the exception of an initial heating step before isothermal amplification begins. A number of isothermal amplification methods, each having different considerations and providing different advantages, are known in the art and have been discussed in the literature, e.g., by Zanoli and Spoto, 2013, “Isothermal Amplification Methods for the Detection of Nucleic Acids in Microfluidic Devices,” Biosensors 3: 18-43, and Fakruddin, et al., 2013, “Alternative Methods of Polymerase Chain Reaction (PCR),” Journal of Pharmacy and Bioallied Sciences 5(4): 245-252, each incorporated herein by reference in its entirety. In some instances, any appropriate isothermic amplification method is used. In some instances, the isothermic amplification method used is selected from: Loop Mediated Isothermal Amplification (LAMP); Nucleic Acid Sequence Based Amplification (NASBA); Multiple Displacement Amplification (MDA); Rolling Circle Amplification (RCA); Helicase Dependent Amplification (HDA); Strand Displacement Amplification (SDA); Nicking Enzyme Amplification Reaction (NEAR); Ramification Amplification Method (RAM); and Recombinase Polymerase Amplification (RPA).

In some instances, the amplification method used is LAMP (see, e.g., Notomi, et al., 2000, “Loop Mediated Isothermal Amplification” NAR 28(12): e63 i-vii, and U.S. Pat. No. 6,410,278, “Process for synthesizing nucleic acid” each incorporated by reference herein in its entirety). LAMP is a one-step amplification system using auto-cycling strand displacement deoxyribonucleic acid (DNA) synthesis. In some instances, LAMP is carried out at 60-65° C. for 45-60 min in the presence of a thermostable polymerase, e.g., Bacillus stearothermophilus (Bst) DNA polymerase I, deoxyribonucleotide triphosphate (dNTPs), specific primers and the target DNA template. In some instances, the template is RNA and a polymerase having both reverse transcriptase activity and strand displacement-type DNA polymerase activity, e.g., Bca DNA polymerase, is used, or a polymerase having reverse transcriptase activity is used for the reverse transcriptase step and a polymerase not having reverse transcriptase activity is used for the strand displacement-DNA synthesis step.

In some instances, the amplification reaction is carried out using LAMP, at about 55° C. to about 70° C. In some instances, the LAMP reaction is carried out at 55° C. or greater. In some instances, the LAMP reaction is carried out 70° C. or less. In some instances, the LAMP reaction is carried out at about 55° C. to about 57° C., about 55° C. to about 59° C., about 55° C. to about 60° C., about 55° C. to about 61° C., about 55° C. to about 62° C., about 55° C. to about 63° C., about 55° C. to about 64° C., about 55° C. to about 65° C., about 55° C. to about 66° C., about 55° C. to about 68° C., about 55° C. to about 70° C., about 57° C. to about 59° C., about 57° C. to about 60° C., about 57° C. to about 61° C., about 57° C. to about 62° C., about 57° C. to about 63° C., about 57° C. to about 64° C., about 57° C. to about 65° C., about 57° C. to about 66° C., about 57° C. to about 68° C., about 57° C. to about 70° C., about 59° C. to about 60° C., about 59° C. to about 61° C., about 59° C. to about 62° C., about 59° C. to about 63° C., about 59° C. to about 64° C., about 59° C. to about 65° C., about 59° C. to about 66° C., about 59° C. to about 68° C., about 59° C. to about 70° C., about 60° C. to about 61° C., about 60° C. to about 62° C., about 60° C. to about 63° C., about 60° C. to about 64° C., about 60° C. to about 65° C., about 60° C. to about 66° C., about 60° C. to about 68° C., about 60° C. to about 70° C., about 61° C. to about 62° C., about 61° C. to about 63° C., about 61° C. to about 64° C., about 61° C. to about 65° C., about 61° C. to about 66° C., about 61° C. to about 68° C., about 61° C. to about 70° C., about 62° C. to about 63° C., about 62° C. to about 64° C., about 62° C. to about 65° C., about 62° C. to about 66° C., about 62° C. to about 68° C., about 62° C. to about 70° C., about 63° C. to about 64° C., about 63° C. to about 65° C., about 63° C. to about 66° C., about 63° C. to about 68° C., about 63° C. to about 70° C., about 64° C. to about 65° C., about 64° C. to about 66° C., about 64° C. to about 68° C., about 64° C. to about 70° C., about 65° C. to about 66° C., about 65° C. to about 68° C., about 65° C. to about 70° C., about 66° C. to about 68° C., about 66° C. to about 70° C., or about 68° C. to about 70° C. In some instances, the LAMP reaction is carried out at about 55° C., about 57° C., about 59° C., about 60° C., about 61° C., about 62° C., about 63° C., about 64° C., about 65° C., about 66° C., about 68° C., or about 70° C.

In some instances, the amplification reaction is carried out using LAMP, for about 30 to about 90 minutes. In some instances, the LAMP reaction is carried out for at least about 30 minutes. In some instances, the LAMP reaction is carried out for at most about 90 minutes. In some instances, the LAMP reaction is carried out for about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 90 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 65 minutes, about 35 minutes to about 70 minutes, about 35 minutes to about 75 minutes, about 35 minutes to about 80 minutes, about 35 minutes to about 90 minutes, about 40 minutes to about 45 minutes, about 40 minutes to about 50 minutes, about 40 minutes to about 55 minutes, about 40 minutes to about 60 minutes, about 40 minutes to about 65 minutes, about 40 minutes to about 70 minutes, about 40 minutes to about 75 minutes, about 40 minutes to about 80 minutes, about 40 minutes to about 90 minutes, about 45 minutes to about 50 minutes, about 45 minutes to about 55 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 90 minutes, about 50 minutes to about 55 minutes, about 50 minutes to about 60 minutes, about 50 minutes to about 65 minutes, about 50 minutes to about 70 minutes, about 50 minutes to about 75 minutes, about 50 minutes to about 80 minutes, about 50 minutes to about 90 minutes, about 55 minutes to about 60 minutes, about 55 minutes to about 65 minutes, about 55 minutes to about 70 minutes, about 55 minutes to about 75 minutes, about 55 minutes to about 80 minutes, about 55 minutes to about 90 minutes, about 60 minutes to about 65 minutes, about 60 minutes to about 70 minutes, about 60 minutes to about 75 minutes, about 60 minutes to about 80 minutes, about 60 minutes to about 90 minutes, about 65 minutes to about 70 minutes, about 65 minutes to about 75 minutes, about 65 minutes to about 80 minutes, about 65 minutes to about 90 minutes, about 70 minutes to about 75 minutes, about 70 minutes to about 80 minutes, about 70 minutes to about 90 minutes, about 75 minutes to about 80 minutes, about 75 minutes to about 90 minutes, or about 80 minutes to about 90 minutes. In some instances, the LAMP reaction is carried out for about 30 minutes, about 35 minutes, about 40 minutes, about 45 minutes, about 50 minutes, about 55 minutes, about 60 minutes, about 65 minutes, about 70 minutes, about 75 minutes, about 80 minutes, or about 90 minutes.

In some instances, the amplification method is Nucleic Acid Sequence Based Amplification (NASBA). NASBA (also known as 3SR, and transcription-mediated amplification) is an isothermal transcription-based RNA amplification system. Three enzymes (avian myeloblastosis virus reverse transcriptase, RNase H and T7 DNA dependent RNA polymerase) are used to generate single-stranded RNA. In certain cases NASBA can be used to amplify DNA. The amplification reaction is performed at 41° C., maintaining constant temperature, typically for about 60 to about 90 minutes (see, e.g., Fakruddin, et al., 2012, “Nucleic Acid Sequence Based Amplification (NASBA) Prospects and Applications,” Int. J. of Life Science and Pharma Res. 2(1):L106-L121, incorporated by reference herein).

In some instances, the NASBA reaction is carried out at about 40° C. to about 42° C. In some instances, the NASBA reaction is carried out at 41° C. In some instances, the NASBA reaction is carried out at most at about 42° C. In some instances, the NASBA reaction is carried out at about 40° C. to about 41° C., about 40° C. to about 42° C., or about 41° C. to about 42° C. In some instances, the NASBA reaction is carried out at about 40° C., about 41° C., or about 42° C.

In some instances, the amplification reaction is carried out using NASBA, for about 45 to about 120 minutes. In some instances, the NASBA reaction is carried out for about 30 minutes to about 120 minutes. In some instances, the NASBA reaction is carried out for at least about 30 minutes. In some instances, the NASBA reaction is carried out for at most about 120 minutes. In some instances, the NASBA reaction is carried out for up to 180 minutes. In some instances, the NASBA reaction is carried out for about 30 minutes to about 45 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 85 minutes, about 30 minutes to about 90 minutes, about 30 minutes to about 95 minutes, about 30 minutes to about 100 minutes, about 30 minutes to about 120 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 85 minutes, about 45 minutes to about 90 minutes, about 45 minutes to about 95 minutes, about 45 minutes to about 100 minutes, about 45 minutes to about 120 minutes, about 60 minutes to about 65 minutes, about 60 minutes to about 70 minutes, about 60 minutes to about 75 minutes, about 60 minutes to about 80 minutes, about 60 minutes to about 85 minutes, about 60 minutes to about 90 minutes, about 60 minutes to about 95 minutes, about 60 minutes to about 100 minutes, about 60 minutes to about 120 minutes, about 65 minutes to about 70 minutes, about 65 minutes to about 75 minutes, about 65 minutes to about 80 minutes, about 65 minutes to about 85 minutes, about 65 minutes to about 90 minutes, about 65 minutes to about 95 minutes, about 65 minutes to about 100 minutes, about 65 minutes to about 120 minutes, about 70 minutes to about 75 minutes, about 70 minutes to about 80 minutes, about 70 minutes to about 85 minutes, about 70 minutes to about 90 minutes, about 70 minutes to about 95 minutes, about 70 minutes to about 100 minutes, about 70 minutes to about 120 minutes, about 75 minutes to about 80 minutes, about 75 minutes to about 85 minutes, about 75 minutes to about 90 minutes, about 75 minutes to about 95 minutes, about 75 minutes to about 100 minutes, about 75 minutes to about 120 minutes, about 80 minutes to about 85 minutes, about 80 minutes to about 90 minutes, about 80 minutes to about 95 minutes, about 80 minutes to about 100 minutes, about 80 minutes to about 120 minutes, about 85 minutes to about 90 minutes, about 85 minutes to about 95 minutes, about 85 minutes to about 100 minutes, about 85 minutes to about 120 minutes, about 90 minutes to about 95 minutes, about 90 minutes to about 100 minutes, about 90 minutes to about 120 minutes, about 95 minutes to about 100 minutes, about 95 minutes to about 120 minutes, or about 100 minutes to about 120 minutes. In some instances, the NASBA reaction is carried out for about 30 minutes, about 45 minutes, about 60 minutes, about 65 minutes, about 70 minutes, about 75 minutes, about 80 minutes, about 85 minutes, about 90 minutes, about 95 minutes, about 100 minutes, about 120 minutes, about 150 minutes, or about 180 minutes.

In some instances, the amplification method is Strand Displacement Amplification (SDA). SDA is an isothermal amplification method that uses four different primers. A primer containing a restriction site (a recognition sequence for HincII exonuclease) is annealed to the DNA template. An exonuclease-deficient fragment of Escherichia coli DNA polymerase 1 (exo-Klenow) elongates the primers. Each SDA cycle consists of (1) primer binding to a displaced target fragment, (2) extension of the primer/target complex by exo-Klenow, (3) nicking of the resultant hemiphosphothioate HincII site, (4) dissociation of HincII from the nicked site and (5) extension of the nick and displacement of the downstream strand by exo-Klenow.

In some instances, the amplification method is Multiple Displacement Amplification (MDA). The MDA is an isothermal, strand-displacing method based on the use of the highly processive and strand-displacing DNA polymerase from bacteriophage Ø29, in conjunction with modified random primers to amplify the entire genome with high fidelity. It has been developed to amplify all DNA in a sample from a very small amount of starting material. In MDA Ø29 DNA polymerase is incubated with dNTPs, random hexamers and denatured template DNA at 30° C. for 16 to 18 hours and the enzyme must be inactivated at high temperature (65° C.) for 10 min. No repeated recycling is required, but a short initial denaturation step, the amplification step, and a final inactivation of the enzyme are needed.

In some instances, the amplification method is Rolling Circle Amplification (RCA). RCA is an isothermal nucleic acid amplification method which allows amplification of the probe DNA sequences by more than 10⁹ fold at a single temperature, typically about 30° C. Numerous rounds of isothermal enzymatic synthesis are carried out by Ø29 DNA polymerase, which extends a circle-hybridized primer by continuously progressing around the circular DNA probe. In some instances, the amplification reaction is carried out using RCA, at about 28° C. to about 32° C.

In some instances, sampling devices and systems disclosed herein comprise at least one oligonucleotide primer, wherein the oligonucleotide primer has a sequence complementary to or corresponding to a Y chromosome sequence. In some instances, sampling devices and systems disclosed herein comprise a pair of oligonucleotide primers, wherein the pair of oligonucleotide primers have sequences complementary to or corresponding to a Y chromosome sequence. In some instances, sampling devices and systems disclosed herein comprise at least one oligonucleotide primer, wherein the oligonucleotide primer comprises a sequence complementary to or corresponding to a Y chromosome sequence. In some instances, sampling devices and systems disclosed herein comprise a pair of oligonucleotide primers, wherein the pair of oligonucleotide primers comprise sequences complementary to or corresponding to a Y chromosome sequence. In some instances, sampling devices and systems disclosed herein comprise at least one oligonucleotide primer, wherein the oligonucleotide primer consists of a sequence complementary to or corresponding to a Y chromosome sequence. In some instances, sampling devices and systems disclosed herein comprise a pair of oligonucleotide primers, wherein the pair of oligonucleotide primers consists of sequences complementary to or corresponding to a Y chromosome sequence. In some instances, the sequence(s) complementary to or corresponding to a Y chromosome sequence is at least 75% homologous to a wild-type human Y chromosome sequence. In some instances, the sequence(s) complementary to or corresponding to a Y chromosome sequence is at least 80% homologous to a wild-type human Y chromosome sequence. In some instances, the sequence(s) complementary to or corresponding to a Y chromosome sequence is at least 85% homologous to a wild-type human Y chromosome sequence. In some instances, the sequence(s) complementary to or corresponding to a Y chromosome sequence is at least 80% homologous to a wild-type human Y chromosome sequence. In some instances, the sequence(s) complementary to or corresponding to a Y chromosome sequence is at least 90% homologous to a wild-type human Y chromosome sequence. In some instances, the sequence(s) complementary to or corresponding to a Y chromosome sequence is at least 95% homologous to a wild-type human Y chromosome sequence. In some instances, the sequence(s) complementary to or corresponding to a Y chromosome sequence is at least 97% homologous to a wild-type human Y chromosome sequence. In some instances, the sequence(s) complementary to or corresponding to a Y chromosome sequence is 100% homologous to a wild-type human Y chromosome sequence.

In some instances, sampling devices and systems disclosed herein are capable of tagging at least a portion of the cell-free nucleic acids (e.g., the amplified cfDNA). In some instances, the tagging comprises: (a) generating ligation competent cell-free DNA by one or more steps comprising: (i) generating a blunt end of the cell-free DNA, In some embodiments, a 5′ overhang or a 3′ recessed end is removed using one or more polymerase and one or more exonuclease; (ii) dephosphorylating the blunt end of the cell-free DNA; (iii) contacting the cell-free DNA with a crowding reagent thereby enhancing a reaction between the one or more polymerases, one or more exonucleases, and the cell-free DNA; or (iv) repairing or remove DNA damage in the cell-free DNA using a ligase; and (b) ligating the ligation competent cell-free DNA to adaptor oligonucleotides by contacting the ligation competent cell-free DNA to adaptor oligonucleotides in the presence of a ligase, crowding reagent, and/or a small molecule enhancer. In some embodiments, the methods further comprise pooling two or more biological samples, each sample obtained from a different subject. In some embodiments, the methods further comprise contacting the biological sample with a white blood cell stabilizer following obtaining the biological sample from the subject. In some embodiments, the one or more polymerases comprises T4 DNA polymerase or DNA polymerase I. In some embodiments, the one or more exonucleases comprises T4 polynucleotide kinase or exonuclease III. In some embodiments, the ligase comprises T3 DNA ligase, T4 DNA ligase, T7 DNA ligase, Taq Ligase, Ampligase, E. coli Ligase, or Sso7-ligase fusion protein. In some embodiments, the crowding reagent comprises polyethylene glycol (PEG), glycogen, or dextran, or a combination thereof. In some embodiments, the small molecule enhancer comprises dimethyl sulfoxide (DMSO), polysorbate 20, formamide, or a diol, or a combination thereof. In some embodiments, ligating in (b) comprises blunt end ligating, or single nucleotide overhang ligating. In some embodiments, the adaptor oligonucleotides comprise Y shaped adaptors, hairpin adaptors, stem loop adaptors, degradable adaptors, blocked self-ligating adaptors, or barcoded adaptors, or a combination thereof.

### Nucleic Acid Detector

In some instances, sampling devices and systems disclosed herein comprise a nucleic acid detector. In some instances, the nucleic acid detector comprises a nucleic acid sequencer. In some instances, sampling devices and systems disclosed herein are configured to amplify nucleic acids and sequence the resulting amplified nucleic acids. In some instances, sampling devices and systems disclosed herein are configured to sequence nucleic acids without amplifying nucleic acids. In some instances, sampling devices and systems disclosed herein comprise a nucleic acid sequencer, but do not comprise a nucleic acid amplifying reagent or nucleic acid amplifying component. In some instances, the nucleic acid sequencer comprises a signal detector that detects a signal that reflects successful amplification or unsuccessful amplification. In some instances, the nucleic acid sequencer is the signal detector. In some instances, the signal detector comprises the nucleic acid sequencer.

In some instances, the nucleic acid sequencer has a communication connection with an electronic device that analyzes sequencing reads from the nucleic acid sequencer. In some instances the communication connection is hard wired. In some instances the communication connection is wireless. For example, a mobile device app or computer software, such as those disclosed herein, can receive the sequencing reads, and based on the sequencing reads, display or report genetic information about the sample (e.g., presence of a disease/infection, response to a drug, genetic abnormality or mutation of a fetus).

In some instances, the nucleic acid sequencer comprises a nanopore sequencer. In some instances, the nanopore sequencer comprises a nanopore. In some instances, the nanopore sequencer comprises a membrane and solutions that create a current across the membrane and drive movement of charged molecules (e.g., nucleic acids) through the nanopore. In some instances, the nanopore sequencer comprises a transmembrane protein, a portion thereof, or a modification thereof. In some instances, the transmembrane protein is a bacterial protein. In some instances, the transmembrane protein is not a bacterial protein. In some instances, the nanopore is synthetic. In some instances, the nanopore performs solid state nanopore sequencing. In some instances, the nanopore sequencer is described as pocket-sized, portable, or roughly the size of a cell phone. In some instances, the nanopore sequencer is configured to sequence at least one of RNA and DNA. Non-limiting examples of nanopore sequencing devices include Oxford Nanopore Technologies MinION and SmidgION nanopore sequencing USB devices. Both of these devices are small enough to be handheld. Nanopore sequencing devices and components are further described in reviews by Howorka (Nat Nanotechnol. 2017 Jul. 6; 12(7):619-630), and Garrido-Cardenas et al. (Sensors (Basel). 2017 Mar. 14; 17(3)), both incorporated herein by reference. Other non-limiting examples of nanopore sequencing devices are offered by Electronic Biosciences, Two Pore Guys, Stratos, and Agilent (technology originally from Genia).

In some instances, the nucleic acid detector comprises reagents and components required for bisulfate sequencing to detect epigenetic modifications. For instance, a long region with many methylation markers can be fragmented. Here, each fragment carrying a methylation marker can be an independent signal. Signals from all the fragments are sufficient in combination to obtain useful genetic information.

In some instances, the nucleic acid detector does not comprise a nucleic acid sequencer. In some instances, the nucleic acid detector is configured to count tagged nucleic acids, wherein the nucleic acid detector quantifies a collective signal from one or more tags.

### Epigenetic Modification Detector

Generally, sampling devices and systems disclosed herein are capable of detecting epigenetic modifications in a genome of a user. In some sampling devices and systems disclosed herein are configured to perform steps comprising: (a) obtaining about 1-100 microliters (μl) of a biological sample from a subject comprising deoxyribose nucleic acid (DNA); and (b) detecting an epigenetic modification of the DNA. In some embodiments, the epigenetic modification comprises DNA methylation at a genetic locus, a histone methylation, histone, ubiquitination, histone acetylation, histone phosphorylation, micro RNA (miRNA). In some embodiments, the DNA methylation comprises CpG methylation or CpH methylation. In some embodiments, the genetic locus comprises a promoter or regulatory element of a gene. In some embodiments, the genetic locus comprises a variable long terminal repeat (LTR). In some embodiments, the genetic locus comprises a cell-free DNA or fragment thereof. In some embodiments, the genetic locus comprises a single nucleotide polymorphism (SNP). In some embodiments, histone acetylation is indicated by a presence or level of histone deacetylases. In some embodiments, the histone modification is at a histone selected from the group consisting of histone 2A (H2A), histone 2B (H2B, histone 3 (H3), and histone 4 (H4). In some embodiments, the histone methylation is methylation of H3 lysine 4 (H3K4me2). In some embodiments, the histone acetylation is deacetylation at H4. In some embodiments, the miRNA are selected from the group consisting of miR-21, miR-126, mi-R142, mi-R146a, mi-R12a, mi-R181a, miR-29c, miR-29a, miR-29b, miR-101, miRNA-155, and miR-148a.

### Capture and Detection

In some instances, sampling devices and systems disclosed herein comprise at least one of a nucleic acid detector, capture component, signal detector, a detection reagent, or a combination thereof, for detecting a nucleic acid in the biological sample. In some instances, the capture component and the signal detector are integrated. In some instances, the capture component comprises a solid support. In some instances the solid support comprises a bead, a chip, a strip, a membrane, a matrix, a column, a plate, or a combination thereof.

In some instances, sampling devices and systems disclosed herein comprise at least one probe for an epigenetically modified region of a chromosome or fragment thereof. In some instances, the epigenetic modification of the epigenetically modified region of a chromosome is indicative of gender or a marker of gender. In some instances, sampling devices and systems disclosed herein comprise at least one probe for a paternally inherited sequence that is not present in the maternal DNA. In some instances, sampling devices and systems disclosed herein comprise at least one probe for a paternally inherited single nucleotide polymorphism. In some instances, the chromosome is a Y chromosome. In some instances, the chromosome is an X chromosome. In some instances, the chromosome is a Y chromosome. In some instances, the chromosome is an autosome. In some instances, the probe comprises a peptide, an antibody, an antigen binding antibody fragment, a nucleic acid or a small molecule.

In some instances, sampling devices and systems comprise a sample purifier disclosed herein and a capture component disclosed herein. In some instances, the sample purifier comprises the capture component. In some instances, the sample purifier and the capture component are integrated. In some instances, the sample purifier and the capture component are separate.

In some instances, the capture component comprises a binding moiety described herein. In some instances, the binding moiety is present in a lateral flow assay. In some instances, the binding moiety is added to the sample before the sample is added to the lateral flow assay. In some instances, the binding moiety comprises a signaling molecule. In some instances, the binding moiety is physically associated with a signaling molecule. In some instances, the binding moiety is capable of physically associating with a signaling molecule. In some instances, the binding moiety is connected to a signaling molecule. Non-limiting examples of signaling molecules include a gold particle, a fluorescent particle, a luminescent particle, and a dye molecule. In some instances the capture component comprises a binding moiety that is capable of interacting with an amplification product described herein. In some instances the capture component comprises a binding moiety that is capable of interacting with a tag on an amplification product described herein.

In some instances, sampling devices and systems disclosed herein comprise a detection system. In some instances, the detection system comprises a signal detector. Non-limiting examples of a signal detector include a fluorescence reader, a colorimeter, a sensor, a wire, a circuit, a receiver. In some instances, the detection system comprises a detection reagent. Non-limiting examples of a detection reagent include a fluorophore, a chemical, a nanoparticle, an antibody, and a nucleic acid probe. In some instances, the detection system comprises a pH sensor and a complementary metal-oxide semiconductor, which can be used to detect changes in pH. In some instances, production of an amplification product by devices, systems, kits or methods disclosed herein changes the pH, thereby indicating genetic information.

In some instances, the detection system comprises a signal detector. In some instances, the signal detector is a photodetector that detects photons. In some instances, the signal detector detects fluorescence. In some instances, the signal detector detects a chemical or compound. In some instances, the signal detector detects a chemical that is released when the amplification product is produced. In some instances, the signal detector detects a chemical that is released when the amplification product is added to the detection system. In some instances, the signal detector detects a compound that is produced when the amplification product is produced. In some instances, the signal detector detects a compound that is produced when the amplification product is added to the detection system.

In some instances, the signal detector detects an electrical signal. In some instances, the signal detector comprises an electrode. In some instances, the signal detector comprises a circuit a current, or a current generator. In some instances, the circuit or current is provided by a gradient of two or more solutions or polymers. In some instances, the circuit or current is provided by an energy source (e.g., battery, cell phone, wire from electrical outlet). In some instances, nucleic acids, amplification products, chemicals or compounds disclosed herein provide an electrical signal by disrupting the current and the signal detector detects the electrical signal.

In some instances, the signal detector detects light. In some instances, the signal detector comprises a light sensor. In some instances, the signal detector comprises a camera. In some instances, the signal detector comprises a cell phone camera or a component thereof.

In some instances, the signal detector comprises a nanowire that detects the charge of different bases in nucleic acids. In some instances, the nanowire has a diameter of about 1 nm to about 99 nm. In some instances, the nanowire has a diameter of about 1 nm to about 999 nm. In some instances, the nanowire comprises an inorganic molecule, e.g., nickel, platinum, silicon, gold, zinc, graphene, or titanium. In some instances, the nanowire comprises an organic molecule (e.g., a nucleotide).

In some instances, the detection system comprises an assay assembly, wherein the assay assembly is capable of detecting a target analyte (e.g., nucleic acid amplification product). In some instances, the assay assembly comprises a lateral flow strip, also referred to herein and in the field, as a lateral flow assay, lateral flow test or lateral flow device. In some instances, a lateral flow assay provides a fast, inexpensive, and technically simple method to detect amplification products disclosed herein. Generally, lateral flow assays disclosed herein comprise a porous material or porous matrix that transports a fluid, and a detector that detects the amplification product when it is present. The porous material can comprise a porous paper, a polymer structure, a sintered polymer, or a combination thereof. In some instances, the lateral flow assay transports the biological fluid or portion thereof (e.g., plasma of blood sample). In some instances, the lateral flow assay transports a solution containing the biological fluid or portion thereof. For instance, methods can comprise adding a solution to the biological fluid before or during addition of the sample to the device or system. The solution can comprise a salt, a polymer, or any other component that facilitates transport of the sample and or amplification product through the lateral flow assay. In some instances, nucleic acids are amplified after they have traveled through the lateral flow strip.

In some instances, devices, the detection system comprises a lateral flow device, wherein the lateral flow device comprises multiple sectors or zones, wherein each desired function can be present in a separate sector or zone. In general, in a lateral flow device, a liquid sample, e.g., a body fluid sample as described herein, containing the target analyte moves with or without the assistance of external forces through sectors or zones of the lateral flow device. In some instances, the target analyte moves without the assistance of external forces, e.g., by capillary action. In some instances, the target analyte moves with assistance of external forces, e.g., by facilitation of capillary action by movement of the lateral flow device. Movement can comprise any motion caused by external input, e.g., shaking, turning, centrifuging, applying an electrical field or magnetic field, applying a pump, applying a vacuum, or rocking of the lateral flow device.

In some instances, the lateral flow device is a lateral flow test strip, comprising zones or sectors that are situated laterally, e.g., behind or ahead of each other. In general, a lateral flow test strip allows accessibility of the functional zones or sectors from each side of (e.g., above and below) the test strip as a result of exposure of a large surface area of each functional zone or sector. This facilitates the addition of reagents, including those used in sample purification, or target analyte amplification, and/or detection.

Any suitable lateral flow test strip detection format known to those of skill in the art is contemplated for use in an assay assembly of the present disclosure. Lateral flow test strip detection formats are well known and have been described in the literature. Lateral flow test strip assay formats are generally described by, e.g., Sharma et al., (2015) Biosensors 5:577-601, incorporated by reference herein in its entirety. Detection of nucleic acids using lateral flow test strip sandwich assay formats is described by, e.g., U.S. Pat. No. 9,121,849, “Lateral Flow Assays,” incorporated by reference herein in its entirety. Detection of nucleic acids using lateral flow test strip competitive assay formats is described by, e.g., U.S. Pat. No. 9,423,399, “Lateral Flow Assays for Tagged Analytes,” incorporated by reference herein in its entirety.

In some instances, a lateral flow test strip detects the target analyte in a test sample using a sandwich format, a competitive format, or a multiplex detection format. In a traditional sandwich assay format, the detected signal is directly proportional to the amount of the target analyte present in the sample, so that increasing amounts of the target analyte lead to increasing signal intensity. In traditional competitive assay formats, the detected signal has an inverse relationship with the amount of analyte present, and increasing amounts of analyte lead to decreasing signal intensity.

In a lateral flow sandwich format, also referred to as a “sandwich assay,” the test sample typically is applied to a sample application pad at one end of a test strip. The applied test sample flows through the test strip, from the sample application pad to a conjugate pad located adjacent to the sample application pad, where the conjugate pad is downstream in the direction of sample flow. In some instances, the conjugate pad comprises a labeled, reversibly-immobilized probe, e.g., an antibody or aptamer labeled with, e.g., a dye, enzyme, or nanoparticle. A labeled probe-target analyte complex is formed if the target analyte is present in the test sample. This complex then flows to a first test zone or sector (e.g., a test line) comprising an immobilized second probe which is specific to the target analyte, thereby trapping any labeled probe-target analyte complex. In some instances, the intensity or magnitude of signal, e.g., color, at the first test zone or sector is used to indicate the presence or absence, quantity, or presence and quantity of target analyte in the test sample. A second test zone or sector can comprise a third probe that binds to excess labeled probe. If the applied test sample comprises the target analyte, little or no excess labeled probe will be present on the test strip following capture of the target analyte by the labeled probe on the conjugate pad. Consequently, the second test zone or sector will not bind any labeled probe, and little or no signal (e.g., color) at the second test zone or sector is expected to be observed. The absence of signal at the second test zone or sector thus can provide assurance that signal observed in the first test zone or sector is due to the presence of the target analyte.

In some instances, sampling devices and systems disclosed herein comprise a sandwich assay. In some instances, the sandwich assay is configured to receive a biological sample disclosed herein and retain sample components (e.g., nucleic acids, cells, microparticles). In some instances, the sandwich assay is configured to receive a flow solution that flushes non-nucleic acid components of the biological sample (e.g., proteins, cells, microparticles), leaving nucleic acids of the biological sample behind. In some instances, the sandwich assay comprises a membrane that binds nucleic acids to help retain the nucleic acids when the flow solution is applied. Non-limiting examples of a membrane the binds nucleic acids includes chitosan modified nitrocellulose.

Similarly, in a lateral flow competitive format a test sample is applied to a sample application pad at one end of a test strip, and the target analyte binds to a labeled probe to form a probe-target analyte complex in a conjugate pad downstream of the sample application pad. In the competitive format, the first test zone or sector typically comprises the target analyte or an analog of the target analyte. The target analyte in the first test zone or sector binds any free labeled probe that did not bind to the test analyte in the conjugate pad. Thus, the amount of signal observed in the first test zone or sector is higher when there is no target analyte in the applied test sample than when target analyte is present. A second test zone or sector comprises a probe that specifically binds to the probe-target analyte complex. The amount of signal observed in this second test zone or sector is higher when the target analyte is present in the applied test sample.

In a lateral flow test strip multiplex detection format, more than one target analyte is detected using the test strip through the use of additional test zones or sectors comprising, e.g., probes specific for each of the target analytes.

In some instances, the lateral flow device is a layered lateral flow device, comprising zones or sectors that are present in layers situated medially, e.g., above or below each other. In some instances, one or more zones or sectors are present in a given layer. In some instances, each zone or sector is present in an individual layer. In some instances, a layer comprises multiple zones or sectors. In some instances, the layers are laminated. In a layered lateral flow device, processes controlled by diffusion and directed by the concentration gradient are possible driving forces. For example, multilayer analytical elements for fluorometric assay or fluorometric quantitative analysis of an analyte contained in a sample liquid are described in EP0097952, “Multilayer analytical element,” incorporated by reference herein.

A lateral flow device can comprise one or more functional zones or sectors. In some instances, the test assembly comprises 1 to 20 functional zones or sectors. In some instances, the functional zones ore sectors comprise at least one sample purification zone or sector, at least one target analyte amplification zone or sector, at least one target analyte detection zone or sector, and at least one target analyte detection zone or sector.

In some instances, the target analyte is a nucleic acid sequence, and the lateral flow device is a nucleic acid lateral flow assay. In some instances, sampling devices and systems disclosed herein comprise a nucleic acid lateral flow assay, wherein the nucleic acid lateral flow assay comprises nucleic acid amplification function. In some instances, target nucleic acid amplification that is carried out by the nucleic acid amplification function takes place prior to, or at the same time as, detection of the amplified nucleic acid species. In some instances, detection comprises one or more of qualitative, semi-quantitative, or quantitative detection of the presence of the target analyte.

In some instances, sampling devices and systems disclosed herein comprise an assay assembly wherein a target nucleic acid analyte is amplified in a lateral flow test strip to generate a labeled amplification product, or an amplification product that can be labeled after amplification. In some instances, a label is present on one or more amplification primers, or subsequently conjugated to one or more amplification primers, following amplification. In some instances, at least one target nucleic acid amplification product is detected on the lateral flow test strip. For example, one or more zones or sectors on the lateral flow test strip can comprise a probe that is specific for a target nucleic acid amplification product.

In some instances, the sampling devices and systems disclosed herein comprise a detector, wherein the detector comprises a graphene biosensor. Graphene biosensors are described, e.g., by Afsahi et al., in the article entitled, “Novel graphene-based biosensor for early detection of Zika virus infection, Biosensor and Bioelectronics,” (2018) 100:85-88.

In some instances, a detector disclosed herein comprises a nanopore, a nanosensor, or a nanoswitch. For instance, the detector can be capable of nanopore sequencing, a method of transporting a nucleic acid through a nanpore based on an electric current across a membrane, the detector measuring disruptions in the current corresponding to specific nucleotides. A nanoswitch or nanosensor undergoes a structural change upon exposure to the detectable signal. See, e.g., Koussa et al., “DNA nanoswitches: A quantitative platform for gel-based biomolecular interaction analysis,” (2015) Nature Methods, 12(2): 123-126.

In some instances, the detector comprises a rapid multiplex biomarker assay where probes for an analyte of interest are produced on a chip that is used for real-time detection. Thus, there is no need for a tag, label or reporter. Binding of analytes to these probes causes a change in a refractive index that corresponds to a concentration of the analyte. All steps can be automated. Incubations can be not be necessary. Results can be available in less than an hour (e.g., 10-30 minutes). A non-limiting example of such a detector is the Genalyte Maverick Detection System.

### Additional Tests

In some instances, sampling devices and systems disclosed herein comprise additional features, reagents, tests or assays for detection or analysis of biological components besides nucleic acids. By way of non-limiting example, the biological component can be selected from a peptide, a lipid, a fatty acid, a sterol, a carbohydrate, a viral component, a microbial component, and a combination thereof. The biological component can be an antibody. The biological component can be an antibody produced in response to a peptide in the subject. These additional assays can be capable of detecting or analyzing biological components in the small volumes or sample sizes disclosed herein and throughout. An additional test can comprise a reagent capable of interacting with a biological component of interest. Non-limiting examples of such reagents include antibodies, peptides, oligonucleotides, aptamers, and small molecules, and combinations thereof. The reagent can comprise a detectable label. The reagent can be capable of interacting with a detectable label. The reagent can be capable of providing a detectable signal.

Additional tests can require one or more antibodies. For instance, the additional test can comprise reagents or components that provide for performing Immuno-PCR (IPCR). IPCR is a method wherein a first antibody for a protein of interest is immobilized and exposed to a sample. If the sample contains the protein of interest, it will be captured by the first antibody. The captured protein of interest is then exposed to a second antibody that binds the protein of interest. The second antibody has been coupled to a polynucleotide that can be detected by real-time PCR. Alternatively or additionally, the additional test can comprise reagents or components that provide for performing a proximity ligation assay (PLA), wherein the sample is exposed to two antibodies specific for a protein of interest, each antibody comprising an oligonucleotide. If both antibodies bind to the protein of interest, the oligonucleotides of each antibody will be close enough to be amplified and/or detected.

In some instances, sampling devices and systems disclosed herein comprise a pregnancy test to confirm the subject is pregnant. In some instances, sampling devices and systems disclosed herein comprise a test for presence of a Y chromosome or absence of a Y chromosome (gender test). In some instances, sampling devices and systems disclosed herein comprise a test for gestational age.

In some instances, sampling devices and systems disclosed herein comprise a test for multiple pregnancies, e.g., twins or triplets. In some instances, methods disclosed herein quantify (absolute or relative) the total amount of fetal nucleic acids in a maternal sample, and the amount of sequences represented by the various autosomes, X and Y chromosomes to detect if one, both or all fetuses are male or female, euploid or aneuploid, etc.

In some instances, sampling devices and systems disclosed herein comprise a pregnancy test for indicating, detecting or verifying the subject is pregnant. In some instances the pregnancy test comprises a reagent or component for measuring a pregnancy related factor. By way of non-limiting example, the pregnancy related factor can be human chorionic gonadotropin protein (hCG) and the reagent or component for hCG comprising an anti-hCG antibody. Also by way of non-limiting example, the pregnancy related factor can be an hCG transcript and the reagent or component for measuring the hCG transcript is an oligonucleotide probe or primer that hybridizes to the hCG transcript. In some instances, the pregnancy related factor is heat shock protein 10 kDa protein 1, also known as early-pregnancy factor (EPF).

In some instances, sampling devices and systems disclosed herein are capable of conveying the age of the fetus. For example, a signal can be generated from the device or system, wherein the level of the signal corresponds to the amount of hCG in the sample from the subject. This level or strength of the signal can be translated or equivocated with a numerical value representing the amount of hCG in the sample. The amount of hCG can indicate an approximate age of the fetus.

In some instances, sampling devices and systems disclosed herein provide an indication or verification of pregnancy, an indication or verification of gestational age, and an indication or verification of gender. In some instances, sampling devices and systems disclosed herein provide an indication of pregnancy, gestational age, and/or gender with at least about 90% confidence (e.g., 90% of the time, the indication is accurate). In some instances, sampling devices and systems disclosed herein provide an indication of pregnancy, gestational age, and/or gender with at least about 95% confidence. In some instances, sampling devices and systems disclosed herein provide an indication of pregnancy, gestational age, and/or gender with at least about 99% confidence.

### Performance Parameters

In some instances, the sampling devices and systems disclosed herein are operable at one or more temperatures. In some instances, the temperature of a component or reagent of the device system, or kit needs to be altered in order for the device system, or kit to be operable. Generally, sampling devices and systems are considered “operable” when they are capable of providing information conveyed by biomarkers (e.g., RNA/DNA, peptides) in the biological sample. In some instances, temperature(s) at which the devices, systems, kits, components thereof, or reagents thereof are operable are obtained in a common household. By way of non-limiting example, temperature(s) obtained in a common household can be provided by room temperature, a refrigerator, a freezer, a microwave, a stove, an electric hot pot, hot/cold water bath, or an oven.

In some instances, devices, systems, kits, components thereof, or reagents thereof, as described herein, are operable at a single temperature. In some instances, devices, systems, kits, components thereof, or reagents thereof, as described herein, only require a single temperature to be operable. In some instances, devices, systems, kits, components thereof, or reagents thereof, as described herein, only require two temperatures to be operable. In some instances, devices, systems, kits, components thereof, or reagents thereof, as described herein, only require three temperatures to be operable.

In some instances, devices, systems, kits disclosed herein comprises a heating device or a cooling device to allow a user to obtain the at least one temperature. Non-limiting examples of heating devices and cooling devices are pouches or bag of material that can be cooled in a refrigerator or freezer, or microwaved or boiled on a stove top, or plugged into an electrical socket, and subsequently applied to devices disclosed herein or components thereof, thereby transmitting heat to the device or component thereof or cooling the device or component thereof. Another non-limiting example of a heating device is an electrical wire or coil that runs through the device or portion thereof. The electrical wire or coil can be activated by external (e.g. solar, outlet) or internal (e.g., battery, cell phone) power to convey heat to the device or portion thereof. In some instances, devices, systems, kits disclosed herein comprise a thermometer or temperature indicator to assist a user with assessing a temperature within the range of temperatures. Alternatively, or additionally, the user employs a device in a typical home setting (e.g., thermometer, cell phone, etc.) to assess the temperature.

In some instances, temperature at which the devices, systems, kits, components thereof, or reagents thereof are operable at a range of temperatures or at least one temperature that falls within a range of temperatures. In some instances, the range of temperatures is about −50° C. to about 100° C. In some instances, the range of temperatures is about −50° C. to about 90° C. In some instances, the range of temperatures is about −50° C. to about 80° C. In some instances, the range of temperatures is about is about −50° C. to about 70° C. In some instances, the range of temperatures is about −50° C. to about 60° C. In some instances, the range of temperatures is about −50° C. to about 50° C. In some instances, the range of temperatures is about −50° C. to about 40° C. In some instances, the range of temperatures is about −50° C. to about 30° C. In some instances, the range of temperatures is about −50° C. to about 20° C. In some instances, the range of temperatures is about −50° C. to about 10° C. In some instances, the range of temperatures is about 0° C. to about 100° C. In some instances, the range of temperatures is about 0° C. to about 90° C. In some instances, the range of temperatures is about 0° C. to about 80° C. In some instances, the range of temperatures is about is about 0° C. to about 70° C. In some instances, the range of temperatures is about 0° C. to about 60° C. In some instances, the range of temperatures is about 0° C. to about 50° C. In some instances, the range of temperatures is about 0° C. to about 40° C. In some instances, the range of temperatures is about 0° C. to about 30° C. In some instances, the range of temperatures is about 0° C. to about 20° C. In some instances, the range of temperatures is about 0° C. to about 10° C. In some instances, the range of temperatures is about 15° C. to about 100° C. In some instances, the range of temperatures is about 15° C. to about 90° C. In some instances, the range of temperatures is about 15° C. to about 80° C. In some instances, the range of temperatures is about is about 15° C. to about 70° C. In some instances, the range of temperatures is about 15° C. to about 60° C. In some instances, the range of temperatures is about 15° C. to about 50° C. In some instances, the range of temperatures is about 15° C. to about 40° C. In some instances, the range of temperatures is about 15° C. to about 30° C. In some instances, the range of temperatures is about 10° C. to about 30° C. In some instances, devices, systems, kits disclosed herein, including all components thereof, and all reagents thereof, are completely operable at room temperature, not requiring cooling, freezing or heating.

In some instances, sampling devices and systems disclosed herein detect components of the biological sample or products thereof (e.g., amplification products, conjugation products, binding products) within a time range of receiving the biological sample. In some instances, detecting occurs via a signaling molecule described herein. In some instances, the time range is about one second to about one minute. In some instances, the time range is about ten seconds to about one minute. In some instances, the time range is about ten seconds to about one minute. In some instances, the time range is about thirty seconds to about one minute. In some instances, the time range is about 10 seconds to about 2 minutes. In some instances, the time range is about 10 seconds to about 3 minutes. In some instances, the time range is about 10 seconds to about 5 minutes. In some instances, the time range is about 10 seconds to about 10 minutes. In some instances, the time range is about 10 seconds to about 15 minutes. In some instances, the time range is about 10 seconds to about 20 minutes. In some instances, the time range is about 30 seconds to about 2 minutes. In some instances, the time range is about 30 seconds to about 5 minutes. In some instances, the time range is about 30 seconds to about 10 minutes. In some instances, the time range is about 30 seconds to about 15 minutes. In some instances, the time range is about 30 seconds to about 20 minutes. In some instances, the time range is about 30 seconds to about 30 minutes. In some instances, the time range is about 1 minute to about 2 minutes. In some instances, the time range is about 1 minute to about 3 minutes. In some instances, the time range is about 1 minute to about 5 minutes. In some instances, the time range is about 1 minute to about 10 minutes. In some instances, the time range is about 1 minute to about 20 minutes. In some instances, the time range is about 1 minute to about 30 minutes. In some instances, the time range is about 5 minutes to about 10 minutes. In some instances, the time range is about 5 minutes to about 15 minutes. In some instances, the time range is about 5 minutes to about 20 minutes. In some instances, the time range is about 5 minutes to about 30 minutes. In some instances, the time range is about 5 minutes to about 60 minutes. In some instances, the time range is about 30 minutes to about 60 minutes. In some instances, the time range is about 30 minutes to about 2 hours. In some instances, the time range is about 1 hour to about 2 hours. In some instances, the time range is about 1 hour to about 4 hours.

In some instances, sampling devices and systems disclosed herein detect a component of the biological sample or a product thereof (e.g., amplification product, conjugation product, binding product) in less than a given amount of time. In some instances, sampling devices and systems disclosed herein provide an analysis of a component of a biological sample or product thereof in less than a given amount of time. In some instances, the amount of time is less than 1 minute. In some instances, the amount of time is less than 5 minutes. In some instances, the amount of time is less than 10 minutes. In some instances, the amount of time is 15 minutes. In some instances, the amount of time is less than 20 minutes. In some instances, the amount of time is less than 30 minutes. In some instances, the amount of time is less than 60 minutes. In some instances, the amount of time is less than 2 hours. In some instances, the amount of time is less than 8 hours.

### Communication & Information Storage

In general, sampling devices and systems disclosed herein comprise a nucleic acid information output. The nucleic acid information output is configured to communicate genetic information from the sample to the user. In some instances, the nucleic acid information output comprises a communication connection or interface so that genetic information obtained can be shared with others not physically present (e.g., family member, physician, or genetic counselor). The communication connection or interface can also allow for input from other sources. In some instances, sampling devices and systems disclosed herein comprise an interface for receiving information based on the genetic information obtained. The interface or communication connection can also receive non-genetic information from the user (e.g., medical history, medical conditions, age, weight, heart rate, blood pressure, physical activity, etc.). The interface or communication connection can also receive information provided by someone or something other than the user.

By way of non-limiting example, this includes web-based information, information from a medical practitioner, and information from an insurance company. In some instances, sampling devices and systems disclosed herein comprise an interface for communicating information based on the genetic information obtained. In some instances, the interface provides a description of a genetic or chromosomal abnormality. In some instances, the interface provides a list of local contacts, such as doctors, support groups, stores and service providers, which support families of children with a genetic or chromosomal abnormality. In some instances, the interface provides an online listing of products or services that would be useful to children with a genetic or chromosomal abnormality. In some instances, sampling devices and systems disclosed herein comprise an information storage unit, e.g., a computer chip. In some instances, the sampling devices and systems disclosed herein comprise means to store genetic information securely. For example, sampling devices and systems disclosed herein can comprise a data chip or a connection (wired or wireless) to a hard drive, server, database or cloud. Non-limiting examples of interfaces for sampling devices and systems disclosed herein are shown in FIG. 4B and FIGS. 5A-E.

In some instances, the sampling devices and systems disclosed herein are capable of collecting, encrypting, and/or storing information from users in a secure manner. Non-limiting examples of such information include health data, information from their wearables, other tests they have done or will do, demographic information etc.

In some instances, the sampling devices and systems disclosed herein are capable of communicating information about biomarkers in the biological sample to a communication device. In some instances the communication device is capable of being connected to the internet (e.g., via port or wireless connection). In some instances the communication device is connected to the internet. In some instances the communication device is not connected to the internet. In some instances, sampling devices and systems disclosed herein are capable of communicating information about biomarkers in the biological sample through the communication device to the internet. Non-limiting examples of communication devices are cell phones, electronic notepads, and computers.

In some instances, sampling devices and systems disclosed herein comprise a communication connection or a communication interface. In some embodiments, the communication interface provides a wired interface. In further embodiments, the wired communications interface utilizes Universal Serial Bus (USB) (including mini-USB, micro-USB, USB Type A, USB Type B, and USB Type C), IEEE 1394 (FireWire), Thunderbolt, Ethernet, and optical interconnect.

In some embodiments, the communication interface provides a wireless interface. See, e.g., FIGS. 5A-E. In further embodiments, the wireless communications interface utilizes a wireless communications protocol such as infrared, near-field communications (NFC) (including RFID), Bluetooth, Bluetooth Low Energy (BLE), ZigBee, ANT, IEEE 802.11 (Wi-Fi), Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), or 3G/4G/LTE/5G cellular communication methods.

In some embodiments, sampling devices and systems described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device includes a communication interface (e.g., network adapter) for communicating with one or more peripheral devices, one or more distinct digital processing devices, one or more computing systems, one or more computer networks, and/or one or more communications networks.

In some embodiments, the digital processing device is communicatively coupled to a computer network (“network”) with the aid of the communication interface. Suitable networks include, a personal area network (PAN), a local area networks (LAN), a wide area network (WAN), an intranet, an extranet, the Internet (providing access to the World Wide Web) and combinations thereof. The network in some cases is a telecommunication and/or data network. The network, in various cases, includes one or more computer servers, which enable distributed computing, such as cloud computing. The network, in some cases and with the aid of the device, implements a peer-to-peer network, which enables devices coupled to the device to behave as a client or a server.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, fitness trackers, smart watches, mobile smartphones, tablet computers, and personal digital assistants. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. In some instances, the operating system comprises an Internet of Things (IoT) device. Non-limiting examples of an IoT device include Amazon's Alexa®, Microsoft's Cortana®, Apple Home Pod®, and Google Speaker®. In some instances, sampling devices and systems disclosed herein comprise a virtual reality and/or augmented reality system.

In some embodiments, sampling devices and systems disclosed herein comprise a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Terminologies

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term ‘about’ a number refers to that number plus or minus 10%, 5%, or 1% of that number, including incrememebts therein. The term “about” when used in the context of a range refers to that range minus 10%, 5%, 1%, or an increment therein, of its lowest value and plus 10% of its greatest value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term, “accuracy,” should be given its broadest definition in light of the specification. However, the term “accuracy” may be used to refer to a statistical measure of how well a binary classification test correctly identifies or excludes a condition. As used herein, the term “accuracy” may also refer to the proportion of true results (both true positives and true negatives) among all samples examined. As used herein, the term “accuracy” may encompass “Rand accuracy” or accuracy as determined by the “Rand index.”

As used herein, the term “analyte” refers to a substance that is measured. In some embodiments, the analyte is a biochemical marker, such as a hormone, a lipid, a carbohydrate, or the like. In some embodiments, the analyte is an external marker, such as a drug metabolite that, in some cases, can be identified in a sample obtained from a subject (e.g., in blood, or urine). Non-limiting examples of analytes include a hormone, a lipid, a carbohydrate, a metabolite, a drug metabolite, a protein, a peptide, DNA, RNA, an epigenetic marker, a pathogen, a microbe, or a portions thereof.

As used herein, the term “biomarker” generally refers to any marker of a subject's biology or condition. A biomarker may be an indicator or result of a disease or condition. A biomarker may be an indicator of health. A biomarker may be an indicator of a genetic abnormality or inherited condition. A biomarker may be a circulating biomarker (e.g., found in a biological fluid such as blood). A biomarker may be a tissue biomarker (e.g., found in a solid organ such as liver or bone marrow). Non-limiting examples of biomarkers include nucleic acids, epigenetic modifications, proteins, peptides, antibodies, antibody fragments, lipids, fatty acids, sterols, polysaccharides, carbohydrates, viral particles, microbial particles. In some cases, biomarkers may even include whole cells or cell fragments.

In general, the term “cell-free nucleic acid,” refers to a polynucleotide or a nucleic acid that can be isolated from a sample without extracting the polynucleotide or nucleic acid from a cell. A cell-free nucleic acid may comprise DNA. A cell-free nucleic acid may comprise RNA.

As used herein, the term “cellular nucleic acid” refers to a polynucleotide that is contained in a cell or released from a cell due to manipulation of the biological sample. Non-limiting examples of manipulation of the biological sample include centrifuging, vortexing, shearing, mixing, lysing, and adding a reagent (e.g., detergent, buffer, salt, enzyme) to the biological sample that is not present in the biological sample when it is obtained. In some instances, the cellular nucleic acid is a nucleic acid that has been released from a cell due to disruption or lysis of the cell by a machine, human or robot. In some instances, cellular nucleic acids (nucleic acids contained by cells) are intentionally or unintentionally released from cells by devices and methods disclosed herein. However, these are not considered “cell-free nucleic acids,” as the term is used herein. In some instances, devices, systems, kits and methods disclosed herein provide for analyzing cell-free nucleic acids in biological samples, and in the process analyze cellular nucleic acids as well.

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

As used herein, the term “cloud” refers to shared or sharable storage of electronic data. The cloud may be used for archiving electronic data, sharing electronic data, and analyzing electronic data.

As used herein, the term “genetic information” generally refers to one or more nucleic acid sequences. In some instances, genetic information may be a single nucleotide or amino acid. For example, genetic information could be the presence (or absence) of a single nucleotide polymorphism. Unless specified otherwise, the term “genetic information” may also refer to epigenetic modification patterns, gene expression data, and protein expression data. In some instances, the presence, absence or quantity of a biomarker provides genetic information. For instance, cholesterol levels may be indicative of a genetic form of hypercholesterolemia. Thus, genetic information should not be limited to nucleic acid sequences.

As used herein, the term, “genetic mutation,” generally refers to an alteration of a nucleotide sequence of a genome. A genetic mutation is different from natural variation or allelic differences. A genetic mutation may be a single nucleotide polymorphism (SNP) or single nucleotide variation (SNV), used interchangeably herein, or an indel.

As used herein, the term “genomic equivalent” generally refers to the amount of DNA necessary to be present in a purified sample to guarantee that all genes will be present.

As used herein, the terms “homologous,” “homology,” or “percent homology” describe sequence similarity of a first amino acid sequence or a nucleic acid sequence relative to a second amino acid sequence or a nucleic acid sequence. In some instances, homology can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application. In some cases, 2 or more sequences may be homologous if they share at least 20%, 25%, 30%. 35%, 40%, 45% 50%, 55%, 60% identity, 65%, 70%, 75%, 80%, 85%, 90%, 91%, 92%, 93%, 94%, 95%, 96%, 97%, 98%, 99% or higher identity when compared and aligned for maximum correspondence over a comparison window, or designated region as measured using one of the following sequence comparison algorithms or by manual alignment and visual inspection. In some cases, 2 or more sequences may be homologous if they share at most 20%, 25%, 30%. 35%, 40%, 45% 50%, 55%, 60% identity, 65%, 70%, 75%, 80%, 85%, 90%, 91%, 92%, 93%, 94%, 95%, 96%, 97%, 98%, 99% or higher identity. Preferably, the % identity or homology exists over a region that is at least 16 amino acids or nucleotides in length or in some cases over a region that is about 50 to about 100 amino acids or nucleotides in length. In some cases, the % identity or homology exists over a region that is about 100 to about 1000 amino acids or nucleotides in length. In some cases, 2 or more sequences may be homologous and share at least 20% identity over at least 100 amino acids in a sequence. For sequence comparison, generally one sequence acts as a reference sequence, to which test sequences may be compared. When using a sequence comparison algorithm, test and reference sequences may be entered into a computer, subsequent coordinates may be designated, if necessary, and sequence algorithm program parameters may be designated. Any suitable algorithm may be used, including but not limited to Smith-Waterman alignment algorithm, Viterbi, Bayesians, Hidden Markov and the like. Default program parameters can be used, or alternative parameters can be designated. The sequence comparison algorithm may then be used to calculate the percent sequence identities for the test sequences relative to the reference sequence, based on the program parameters. Any suitable algorithm may be used, whereby a percent identity is calculated. Some programs for example, calculate percent identity as the number of aligned positions that identical residues, divided by the total number of aligned positions. A “comparison window”, as used herein, includes reference to a segment of any one of the number of contiguous or non-contiguous positions which may range from 10 to 600 positions. In some cases the comparison window may comprise at least 10, 20, 50, 100, 200, 300, 400, 500, or 600 positions. In some cases the comparison window may comprise at most 10, 20, 50, 100, 200, 300, 400, 500, or 600 positions. In some cases the comparison window may comprise at least 50 to 200 positions, or at least 100 to at least 150 positions in which a sequence may be compared to a reference sequence of the same number of contiguous or non-contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al, eds. 1995 supplement)). In some cases, a comparison window may comprise any subset of the total alignment, either contiguous positions in primary sequence, adjacent positions in tertiary space but discontinuous in the primary sequence, or any other subset of 1 up to all residues in the alignment.

The term, “indel,” as used herein refers to an insertion or a deletion of a nucleobase that may differ between the genomes of two members of the same species. In some instances, the indel is mono-, bi-, tri- or tetra-allelic. In some instances, the insertion comprises one nucleobase, two nucleobases, three nucleobases, four nucleobases, five nucleobases, or more.

As used herein, the terms, “isolate,” “purify,” “remove,” “capture,” and “separate,” may all be used interchangeably unless specified otherwise.

As used herein, the terms, “normal individual” and “normal subject” refer to a subject that does not have a condition or disease of interest. For example, if the method or device being described is being used to detect a type of cancer, a normal subject does not have that type of cancer. The normal subject may not have cancer at all. In some instances, the normal subject is not diagnosed with any disease or condition. In some instances, the normal subject does not have a known genetic mutation. In some instances, the normal subject does not have a genetic mutation that results in a detectable phenotype that would distinguish the subject from a normal subject that does not have a known genetic mutation. In some instances, the normal subject is not infected by a pathogen. In some instances, the normal subject is infected by a pathogen, but has no known genetic mutation.

Throughout the application, there is recitation of the phrases “nucleic acid corresponding to a chromosome,” and “sequence corresponding to a chromosome.” As used herein, these phrases are intended to convey that the “nucleic acid corresponding to the chromosome” is represented by a nucleic acid sequence that is identical or homologous to a sequence found in that chromosome. The term “homologous” is described in the foregoing description.

A “sample” as used herein is a biological sample that is derived from a subject. In some embodiments, the sample is obtained directly or indirectly from the subject. In some embodiments, the sample is derived from another sample that has been obtained directly or indirectly from the subject. Non-limiting examples of samples include blood, urine, interstitial fluid, tear fluid, tissue, hair, or sweat, or components thereof.

A “single nucleotide polymorphism” or “SNP,” as used herein, refers to a single nucleotide that may differ between the genomes of two members of the same species. The usage of the term should not imply any limit on the frequency with which each variant occurs. In some instances, the SNP is mono-, bi-, tri- or tetra-allelic.

As used herein, the term “specific to,” refers to a sequence or biomarker that is found only in, on or at the thing that the sequence or biomarker is specific to. For example, if a sequence is specific to a Y chromosome that means that it is only found on the Y chromosome and not on another chromosome.

As used herein, the term, “tag” generally refers to a molecule that can be used to identify, detect or isolate a nucleic acid of interest. The term, “tag,” may be used interchangeably with other terms, such as “label,” “adapter,” “oligo,” and “barcode,” unless specified otherwise. Note, however, that the term, “adapter,” can be used to ligate two ends of a nucleic acid or multiple nucleic acids without acting as a tag.

### Examples

The following illustrative examples are representative of embodiments of the software applications, systems, and methods described herein and are not meant to be limiting in any way.

In the following description, various examples will be described. For purposes of explanation, specific configurations and details are set forth in order to provide a thorough understanding of the examples. However, it will also be apparent to one skilled in the art that the examples can be practiced without the specific details. Furthermore, well-known features can be omitted or simplified in order not to obscure the example being described.

Examples of the present disclosure are directed to, among other things, methods, systems, and computer-readable media for managing presentation of recommendations and notifications using user devices. Generally, recommendations and notifications are presented when certain trigger criteria are met. In some examples, certain recommendations and notifications can be associated with the identification of a biomarker detected from a “sampling device” such that the presence of the biomarker will result in a user receiving one or more corresponding notifications and recommendations.

### Example 1—Fetal Sex Test

In one example, FIG. 1 illustrates an example a computer implemented recommendation platform for managing the presentation of notifications and recommendations, according to at least one example, fetal sex testing. The process can begin at 101A by a user (i.e., pregnant woman) performing a fetal sex test, for e.g., using a sampling device described herein (see Examples 5-7) that identifies the presence of a biomarker (e.g., “sampling device” determining the presence of target circulating fetal cell-free DNA, chromosomal aneuploidy, epigenetic modification, or other biomarker). User sourced information 101C (e.g., biological data such as weight, height, heart rate, blood pressure, other health data; medical information such as doctor, health insurance, other medical information; and lifestyle information such as food intake, exercise, location, or other lifestyle information) and externally sourced data 101A (e.g., electronic medical records, prescription history, or other data) can be utilized by the recommendation engine 108 to inform the recommendation template 102A (e.g., service templates, product templates, health templates, and social templates) and custom templates 102B (e.g., user defined templates, doctor defined templates, company defined templates, or other stakeholder defined templates). The recommendation generator 104 can present a recommendations or notifications based on triggered criteria or events from recommendation or custom templates 106. For example, one or more notifications or recommendations can be presented based on a user defined access (e.g., self, medical professional, family and friends, etc.) to notifications and recommendations, access control engine 105. The recommendations and notifications can include service recommendations (e.g., fitness coach, nutritionist, etc.), product recommendations (e.g., prenatal vitamins, food/drink, exercise accessories, etc.), health recommendations (e.g., lab tests, doctor, etc.), or social recommendations (e.g., support group, etc.). A communication engine can be used to engage and connect pregnant women with other patients, medical professionals, family, friends, or others within the “sampling device” system. For example, the communication engine can enable users to post information about their pregnancy, receive response from other users or medical professionals, and trigger actions based on posting and responses (e.g., alerts to healthcare professionals, recommendations for additional testing, or connection to support groups).

### Example 2—Cancer Test

In another example, FIG. 1 illustrates a simplified block diagram depicting a computer implemented recommendation platform for managing the presentation of notifications and recommendations, according to at least one example, prostate cancer testing. The process can begin at 101B by a user performing a prostate cancer test that identifies the presence of a biomarker (e.g., “sampling device” determining the presence and quantity of prostate-specific antigen, or other biomarker). User sourced information 101C (e.g., biological data such as weight, height, heart rate, blood pressure, other health data; medical information such as doctor, health insurance, other medical information; and lifestyle information such as food intake, exercise, location, or other lifestyle information) and externally sourced data 101A (e.g., electronic medical records, prescription history, or other data) can be utilized by the recommendation engine 103 to inform the recommendation template 102A (e.g., service templates, product templates, health templates, and social templates) and custom templates 110 (e.g., user defined templates, doctor defined templates, company defined templates, or other stakeholder defined templates). The recommendation generator 104 can present a recommendations or notifications based on triggered criteria or events from recommendation or custom templates 107. For example, one or more notifications or recommendations can be presented based on a user defined access (e.g., self, medical professional, family and friends, etc.) to notifications and recommendations, access control engine 105. The recommendations and notifications can include service recommendations (e.g., fitness coach, nutritionist, etc.), product recommendations (e.g., therapeutic, food/drink, exercise accessories, etc.), health recommendations (e.g., lab tests, doctor, etc.), or social recommendations (e.g., support group, etc.). A communication engine can be used to engage and connect cancer patients with other patients, medical professionals, family, friends, or others within the “sampling device” system. For example, the communication engine can enable users to post information about their treatment status, receive response from other users or medical professionals, and trigger actions based on posting and responses (e.g., alerts to healthcare professionals, recommendations for additional testing, or connection to support groups).

### Example 3—Flu Testing

In another example, FIG. 1 illustrates a simplified block diagram depicting an example a computer implemented recommendation platform for managing the presentation of notifications and recommendations, according to at least one example, flu testing. The process can begin at 101B by a user performing a flu test that identifies the presence of a biomarker (e.g., viral DNA, or other biomarker). User sourced information 101C (e.g., biological data such as weight, height, heart rate, blood pressure, other health data; medical information such as doctor, health insurance, other medical information; and lifestyle information such as food intake, exercise, location, or other lifestyle information) and externally sourced data 101A (e.g., electronic medical records, prescription history, or other data) can be utilized by the recommendation engine 108 to inform the recommendation template 102A (e.g., service templates, product templates, health templates, and social templates) and custom templates 102B (e.g., user defined templates, doctor defined templates, company defined templates, or other stakeholder defined templates). The recommendation generator 104 can present a recommendations or notifications based on triggered criteria or events from recommendation or custom templates 107. For example, one or more notifications or recommendations can be presented based on a user defined access (e.g., self, medical professional, family and friends, etc.) to notifications and recommendations, access control engine 105. The recommendations and notifications can include service recommendations (e.g., fitness coach, nutritionist, etc.), product recommendations (e.g., therapeutic, food/drink, exercise accessories, etc.), health recommendations (e.g., lab tests, doctor, etc.), or social recommendations (e.g., support group, etc.). A communication engine can be used to engage and connect flu patients with other patients, medical professionals, family, friends, or others within the “sampling device” system. For example, the communication engine can enable users to post information about their treatment status, receive response from other users or medical professionals, and trigger actions based on posting and responses (e.g., alerts to healthcare professionals, recommendations for additional testing, or connection to support groups).

### Example 4—Sexually Transmitted Disease Testing

In another example, FIG. 1 illustrates a simplified block diagram depicting an a computer implemented recommendation platform for managing the presentation of notifications and recommendations, according to at least one example, sexually transmitted disease testing. The process can begin at 101B by a user performing a Sexually transmitted disease (STD) test that identifies the presence of a biomarker (e.g., HIV, or other biomarker). User sourced information 101C (e.g., biological data such as weight, height, heart rate, blood pressure, other health data; medical information such as doctor, health insurance, other medical information; and lifestyle information such as food intake, exercise, location, or other lifestyle information) and externally sourced data 101A (e.g., electronic medical records, prescription history, or other data) can be utilized by the recommendation engine 103 to inform the recommendation template 102A (e.g., service templates, product templates, health templates, and social templates) and custom templates 102B (e.g., user defined templates, doctor defined templates, company defined templates, or other stakeholder defined templates). The recommendation generator 104 can present a recommendations or notifications based on triggered criteria or events from recommendation or custom templates 107. For example, one or more notifications or recommendations can be presented based on a user defined access (e.g., self, medical professional, family and friends, etc.) to notifications and recommendations, access control engine 116. The recommendations and notifications can include service recommendations (e.g., fitness coach, nutritionist, etc.), product recommendations (e.g., therapeutic, food/drink, exercise accessories, etc.), health recommendations (e.g., lab tests, doctor, etc.), or social recommendations (e.g., support group, etc.). A communication engine can be used to engage and connect user with other patients, medical professionals, family, friends, or others within the “sampling device” system. For example, the communication engine can enable users to post information about their treatment status, receive response from other users or medical professionals, and trigger actions based on posting and responses (e.g., alerts to healthcare professionals, recommendations for additional testing, or connection to support groups).

### Example 5—Sampling Device for Analysis of Cell-Free Nucleic Acids from Whole Blood

A sampling device according to the present disclosure for purifying separating plasma from maternal whole blood for the purpose of analyzing cell-free fetal nucleic acids was constructed. The device consists of 6 layers. From bottom to top these are:

(1) Lower Adhesive Sheet

(2) Lower Separation Disc: 16 mm diameter disc of adhesive sheet material (polymer material that is inert to DNA or Plasma) with glue on the side facing the Lower Adhesive Sheet

(3) Polyethersulfone (PES) membrane, various sizes, typically between 6 and 16 mm, preferred design features 10 mm PES membrane. The membrane serves as wicking material which attracts the plasma from the filter through capillary force.

(4) Filter Disc (e.g., Pall Vivid™ Membrane), 16 mm diameter, rough side facing up, shiny side facing the PES membrane.

(5) Upper Separation Disc: same material as Lower Separation Disc, size 12 or 14 mm diameter, containing a 4 mm hole in the center. When using adhesive sheet material, now the glue side is facing up to meet the Upper Adhesive Sheet. The Upper Separation Disc is smaller than the Filter Disc in diameter. This allows the glue from the Upper Adhesive Sheet to interact with the edges of the Filter Disc and thereby sealing it at the edges.

(6) Upper Adhesive Sheet, a 6 mm hole is punched in the location where the center of the device will be located.

All layers are lined up at their center and then laminated using a standard office lamination machine.

To evaluate the plasma transfer onto the PES membrane, the membrane was weighed before and after application of the plasma to the Disc Filter. The device construction was slightly altered to allow quick removal of the PES membrane. Instead of sandwiching the layers from Upper to Lower Separation Discs between Adhesive Sheets, a set of concentric spacer discs were applied to the top of the device, ensuring a tight fit between the filter and the PES membrane. The Lower Separation Disc was replaced with a parafilm layer. 80 μl of whole blood was applied to the center of the device through the hole in the Upper Adhesive Sheet and the hole in the Upper Separation Disc. This volume was chosen to maximize the amount of plasma transferred onto the PES membrane. However, a volume of plasma (0.5 μl to 1 μl) could have been obtained with 10 μl of blood and this would have been sufficient for Y chromosome detection. The blood distributed centripetally throughout the Filter Disc by capillary forces. Plasma was also wicked through the Filter Disc into the PES membrane by capillary forces. After about two minutes, an average of 6.3 μg of plasma was transferred to the PES membrane, indicating about 6 to 7 μl of plasma had been transferred to the PES membrane as shown in the following Table 1.

TABLE 1

| Blood volume | Weight of the | Weight of the | μg of plasma |
|---|---|---|---|
| applied to | PES/Lower Disc | PES/Lower Disc | in the PES |
| Vivid ™ filter | after filtration in μg | after filtration in μg | membrane |
| 80 | 46.7 | 51 | 4.3 |
| 80 | 52 | 61 | 9 |
| 80 | 53.5 | 59.3 | 5.8 |
| 80 | 59 | 65.3 | 6.3 |
| Average | 52.8 | 59.15 | 6.35 |

With the foregoing results taken in to account, 40 μl of male whole blood were transferred onto a device as described with a 12 mm Upper disc configuration. The PES membrane containing the plasma was transferred into an Eppendorf tube (0.5 ml) and 100 μl of EB buffer (QGEN) was added to elute the DNA on the PES membrane. After elution of the DNA from the membrane, 10 μl of the buffer containing the eluted cfDNA was used directly in a molecular amplification reaction. Real-time recombinase polymerase amplification was performed on the eluted cfDNA with primers specific to a marker on the Y chromosome.

### Example 6—Sampling Device for Analysis of Fetal Cell-Free Nucleic Acids from Maternal Blood

The device consists of multiple layers as exemplified in Example 5.

Application of blood and filtration to the device occurs as follows:

40 μl to 60 μl of whole blood is applied to the center of the device through the hole in the Upper Adhesive Sheet and the hole in the Upper Separation Disc. The blood distributes centripetally throughout the Filter Disc by capillary forces. Plasma is also wicked through the Filter Disc into the PES membrane by capillary forces. After about two minutes, the maximum amount of plasma has been transferred into the PES membrane.

The PES membrane containing cell-free nucleic acids is recovered as follows:

The device is cut out around the edges of the PES membrane. The membrane separates easily from the Filter and the Lower Disc.

DNA is eluted from the membrane as follows:

The PES membrane containing the plasma is transferred into an Eppendorf tube (0.5 ml) and 100 μl of elution buffer are added (elution buffer can be H₂O, EB buffer (QGEN), PBS, TE or others suitable for subsequent molecular analysis). After elution of the DNA from the membrane, the buffer, containing the eluted cfDNA, is used directly in a molecular amplification reaction. Real-time recombinase polymerase amplification was performed on the eluted cfDNA with primers specific to a marker on the Y chromosome.

### Example 7—Real-Time Monitoring of Biological Data

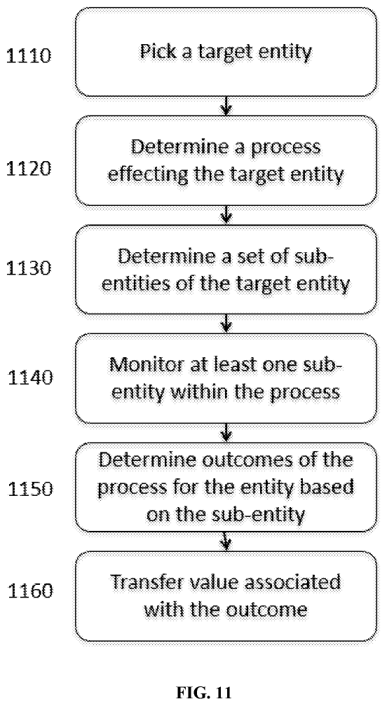

Use of the platform in accordance with various embodiments described herein are provided in FIG. 11. A target entity is picked by the platform 1110. Non-limiting examples of target entities include a human, an animal, an artificial organism, or a bioreactor.

Next, a process of the target entity is determined 1120. Non-limiting examples of processes include, pregnancy, travel, diet, stress test, health protocol, wellness protocol, surgery, augmentation, construction process, or manufacturing process. Step 1120 enables the platform to anticipate and react to lifecycle changes for the target bio-entity selected in 1110. The platform has data associated with other bio-entities that undergone similar changes and has the ability to provision a supply chain of services that enhance outcomes of the changes. Unlike traditional medical services, the processes are not limited to adverse health events and outcomes may include social, business, financial, research and other decisions. When the target entity includes experimental artificial organisms, the changes may involve a novel bio-organism construction procedure, treatment plan, radiation, and the like. When the target entity includes production facilities, such as bioreactors, the changes may include predefined manufacturing process and their modifications.

Next, a set of sub-entities is determined 1130, such as a fetus, microbiome, immune system, an artificial organ, bio prosthesis, cells, organelle, or a tumor. The selection of relevant sub-entities enables the platform to further specify, customize and provision monitoring services, including providers, devices, reagents, protocols, sample size and scope, locations, timing, and the like. The platform determines protocols to monitor and record whether interactions between sub-entities have potentially beneficial, neutral or adverse effects on the parent entity, based on previous records and analysis. This step enables the platform to allocate resources, especially if they involve configuration and provisioning of public/private testing facilities, e.g. multi-cell test lockers. One of the key outputs of Step 1130 is creation of a secure customized instrumented bio-data supply chain capable of producing relevant data necessary to drive the system to desired outcomes.

Next, at least one of the sub-entities is monitored 1140. For example, the DNA, RNA, hormones, metabolic products, rate of cell growth, or concentration of a metabolite or analyte, of the sub-entity is monitored. Various sampling devices may be utilized in step 1140, including the sampling devices described herein. Sampling devices are provided to users in lockers that are maintained by health care professionals to ensure high quality results, and continuous connections to data servers, including video monitoring, security, privacy, authentication, and the like.

Next, outcomes of the process is determined 1150, including for example a baby shower, diet change, travel arrangements, antibiotic replacement, structure modification, environmental conditions, and the like. Processing takes place on, e.g. a server equipped with Machine Learning system that analyzes the results. In some implementations, the platform may recommend the scope of disclosure of the outcomes to third parties.

Pregnancy test->Baby girl shower->female online friends

Diet malfunction->vegan community (reddit or quora)

Veterinary surgery->trusted dog walker

Alzheimer diagnosis->financial planner

In some implementations, the system may request additional data from other devices, e.g. wearables, embeddable devices, monitoring cameras, and like. Depending on service configuration, the consequences can be affected manually, e.g. through user interaction, or automatically, e.g. by launching a mitigating service, which in turn can be treated as Step 1120 and lead to a repeat of the cycle. The data and process outcomes are stored for future use. In some implementations, the data is matched to a population cohort and directed for further protocol optimization, including expansion or reduction of the sub-entity set (step 1130). In some implementation, the system is directed to repeat step 140 in order to collect additional samples. Appropriate notifications are issued to notify the user and configure testing protocols.

Lastly, the value associated with the outcome is transferred 1160. Value in this example includes, for example, payments, points, subscriptions, “likes” on social media, feedback, data contribution commitments, renewals, and the like. Step 1160 can precede or be run in parallel with other steps of the process and distribute value as soon as a particular operation of the services supply chain takes place. to ensure integrity of the process, service provisioning and value distribution associated with Step 160 is performed using distributed ledger technologies, e.g. block chain. In some implementations, value distribution is done via automated contracts based on achieved short- and long-term outcomes. In some implementations, value distribution is done via crediting a financial services account, e.g. insurance, monetary or social credit

### Example 8—Jane Doe Example

In this example, the target entity is a pregnant human female 1110, Jane Doe. Referring to FIG. 11 and Example 7, the platform enables Jane Doe access to a range of services and service providers that offer to enhance certain aspects of her life. In this example, Jane is a frequent business traveler who wants to maintain her vegan diet while traveling to India and taking antibiotics prescribed by her doctor for a urinary infection 1120. When she buys her airline ticket she is automatically enrolled into the platform disclosed herein, which provides Jane with monitoring service to ensure her diet and medication protocol do not have adverse effects on the state of her microbiome 1130 during her trip. During her trip to India, Jane accesses microbiome DNA/RNA test lockers located at public and private places, such as airports, hotels, doctor's office, and the pharmacy. The services sends her secure codes and maintains test lockers that ensure privacy and confidentiality of the transaction. A sampling device is provided in the test lockers to determine a level of an analyte (e.g., a biomarker) 1140. With each test, the outcomes of the process for Jane are generated 1150. In addition, the platform generates a recommendation for restaurants and meals that are vegan. In response to a fluctuation in biomarker level, the platform modifies Jane's medical protocol by referring her to a doctor. The platform also aggregates the biological data with externally coursed data such as from Jane's smartwatch or stress sensor embedded into her AirPods®. Based on the aggregate biological data, the platform generates a recommendation for her daily exercise routine. Upon Jane's return from the trip, the platform sets up her home testing device to capture extended biological data and stores it for analysis and future reference.

### Example 9—Veterinary Clinic Example

In this example, the target entity is Eric, a manager of a veterinary clinic, 1110. Referring to FIG. 11 and Example 7, the platform enables Eric to offer remote services to monitor the health 1120 of pets 1130 for Eric's clients via a subscription. Eric's agent registers the pets with the initial health exam that includes DNA, immunology, microbiome, metabolics, blood and other types of tests 1140. Based on the determined outcomes of the tests 1150, Eric recommends that pet owners perform specific diet and exercise routines with their pets, prescribes treatment and refers clients to local surgeons if necessary 1160. The owners periodically drop off biological samples at contracting locations and get further recommendations 1140. As the pets age, the clinic offers possible replacements, based on breed health profiles, exercise needs, and specific biological characteristics 1150. Next, the value associated with transaction from 1140 is distributed by the platform 1160. In some embodiments, a distributed ledger technology is used.

### Example 10—Biological Laboratory Technician Example

In this example, Jeffry is a biolab technician that builds artificial organs and tests protocols for cancer treatment on the artificial organs. In this example, referring to FIG. 11 and Example 7, the target entity is the artificial organ 1110, and Jeffry is able to monitor the inflammatory response 1120 response of the artificial organ 1110 to various treatments. To determine inflammation, pro-inflammatory markers 1130 are measured continuously 1140. If inflammatory markers increase in response to a given cancer treatment, the platform generates a recommendation that adjusts the treatment protocol 1150. In addition, adverse side effects of the cancer treatment are simultaneously monitored 1140, such as therapeutic non-response, toxicity, and the like 1140. This can be customized to the patient. In some implementations, the platform comprises a multi-unit parallel bio-construction and testing apparatus, e.g. 3D printer, connected to and monitored by the data network. Next, the value associated with transaction from 1140 is distributed by the platform 1160. In some embodiments, a distributed ledger technology is used.

### Example 11—Management of Chronic Illness

A subject with chronic disabilities that cause fatigue, pain, or are limiting to the subject's energy levels utilizes the platform described herein. In this example, the subject may suffer from autoimmune disorders such as arthritis, lupus, and multiple sclerosis; chronic pain disorders due to past physical traumas or surgeries; cancers and their side effects; psychiatric disorders such as major depressive disorder or post-traumatic stress disorder; chronic fatigue syndrome as a result of other disorders such as infections or psychiatric traumas; recovery from surgery or physical injury; or neurological disorders such as chronic intractable migraine. Alternatively, or in addition, the subject may be in recovery from intense exercise.

Daily stress management is critical to establishing and improving wellbeing for these patients. One common method for achieving this is through the use of spoon theory/the spoon metaphor.

Spoon theory requires that one ration their energy expenditure throughout the day under the assumption that only a constant amount is available from day to day. Spoon theory ascribes a unit of energy (“number of spoons”) to typical behaviors (e.g. getting out of bed, working out, going to school). The patient then focuses on not exceeding a certain number of spoons in a day. Some activities require more or less spoons than others, so a patient is asked to proactively manage their spoon consumption throughout the day.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure.

What is claimed is:

1. A computer-implemented platform comprising:
(a) a sampling device configured to:
(i) receive a biologic sample from a user;
(ii) analyze the biologic sample to detect a quantity, a presence, or both of an analyte; and
(b) a mobile processor configured to provide a mobile application, the mobile application comprising:
(i) a user sourced information module receiving user biological data; and
(c) a data processor configured to provide a recommendation application, the recommendation application comprising:
(i) a reception module receiving the user biological data and at least one of the quantity of the analyte or the presence of the analyte;
(ii) a recommendation generation module determining a recommendation based on the user biological data and at least one of the quantity of the analyte or the presence of the analyte; and
(iii) a transmission module transmitting the recommendation to the mobile processor;
wherein at least one of the mobile processor and the data processor are further configured to provide a sample module receiving the quantity of the analyte, the presence of the analyte, or both.

2. The platform of claim 1, wherein at least one of the user sourced information module or the reception module further receive an externally sourced data.

3. The platform of claim 2, wherein the externally sourced data comprises a website, a video, a document file, a medical record, a pharmacy record, a medication history, a health insurance information, a subscription information, metabolic activity data, physical activity data, heart rate data, blood pressure data, metabolite data, sleep data, augmentation data, genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions, or any combination thereof.

4. The platform of claim 1, wherein the recommendation application further comprises a database having a plurality of recommendation templates.

5. The platform of claim 4, wherein the recommendation application further comprises a template selection module selecting at least one recommendation template from the plurality of recommendation templates based on the user biological data and at least one of the quantity of the analyte or the presence of the analyte.

6. The platform of claim 5, wherein the recommendation generation module further determines the recommendation based on the at least one selected recommendation templates.

7. The platform of claim 5, wherein the at least one recommendation template comprises a trigger, a rule, or both.

8. The platform of claim 6, wherein the recommendation is further based on the trigger, the rule, or both.

9. The platform of claim 5, wherein the at least one recommendation template is a pre-defined template or a custom template.

10. The platform of claim 5, wherein the at least one recommendation template is determined by a machine-learning algorithm.

11. The platform of claim 1, wherein the recommendation application further comprises an access control module confirming an access of the recommendation to the user, a third party, or both.

12. The platform of claim 11, wherein the transmission module transmits the recommendation to the user, the one or more service agents, or both based on the confirmation of access.

13. The platform of claim 1, wherein the recommendation generation module determines the recommendation by a machine learning algorithm.

14. The platform of any one of claim 1, wherein the user biological data comprises a weight, blood pressure, height, heart rate, food intake, nutritional history, activity history, sleep history, geolocation, body temperature, step count, body fat percentage, an emergency contact, a family contact, a friend contact, genetic data, genomic data, epigenetic information, microbiome information, proteomic and transcriptomic information, immune repertoire information, pharmacogenetics, blood oxygen levels, travel information, or drug-drug interactions, or any combination thereof.

15. The platform of claim 1, wherein the recommendation comprises a fitness recommendation, nutrition recommendation, mental health recommendation, a recommendation for further testing, or any combination thereof.

16. The platform of claim 1, wherein the sampling device comprises:
(a) a sample purifier for removing a cell from a biological fluid sample to produce a cell-depleted sample; and
(b) at least one of a detection reagent and a signal detector for detecting a plurality of cell-free DNA fragments in the cell-depleted sample.

17. The platform of claim 16, wherein the sample purifier comprises a filter, and wherein the filter has a pore size of about 0.05 microns to about 2 microns.

18. The platform of claim 17, wherein the filter is a vertical filter.

19. The platform of claim 16, wherein the sample purifier comprises a binding moiety selected from an antibody, antigen binding antibody fragment, a ligand, a receptor, a peptide, a small molecule, and a combination thereof.

20. The platform of claim 19, wherein the binding moiety is capable of binding an extracellular vesicle.

21. The platform of claim 16, wherein the at least one nucleic acid amplification reagent comprises an isothermal amplification reagent.

22. The platform of claim 16, wherein the signal detector is a lateral flow strip.

23. The platform of claim 16, wherein the data processor and the sampling device are contained in a single housing.

24. The platform of claim 16, wherein the sampling device is capable of detecting the plurality of biomarkers in the cell-depleted sample within about five minutes to about twenty minutes of receiving the biological fluid.

25. A computer-implemented method comprising:
(a) receiving, by a sampling device, a biologic sample from the user;
(b) analyzing, by the sampling device, the biologic sample to detect a quantity, a presence, or both of an analyte; and
(c) receiving, by a mobile processor, a user biological data;
(d) receiving, by the mobile processor or a data processor, the quantity of the analyte, the presence of the analyte, or both;
(e) receiving, by the data processor, the user biological data and at least one of the quantity of the analyte or the presence of the analyte;
(f) generating, by the data processor, a recommendation based on the user biological data and at least one of the quantity of the analyte or the presence of the analyte; and
(g) transmitting the recommendation to the mobile processor.

26. The method of claim 25, further comprising receiving, by at least one of the user sourced information module an externally sourced data.

27. The method of claim 26, wherein the externally sourced data comprises a website, a video, a document file, a medical record, a pharmacy record, a medication history, a health insurance information, a subscription information, metabolic activity data, physical activity data, heart rate data, blood pressure data, metabolite data, sleep data, augmentation data, genetic data, genomic data, epigenetic information, family history information, microbiome information, pathogen or infectious disease information, vaccination information, proteomic and transcriptomic information, immune repertoire information, pharmacogenetics, medication, drug dosing, or drug-drug interactions or any combination thereof.

28. The method of claim 25, further comprising storing, in a database a plurality of recommendation templates.

29. The method of claim 25, further comprising selecting, by the data processor at least one recommendation template from the plurality of recommendation templates based on the user biological data and at least one of the quantity of the analyte or the presence of the analyte.

30. The method of claim 29, further comprising determining, by the data processor, the recommendation based on the at least one selected recommendation templates.

31. The method of claim 30, wherein the at least one selected recommendation template comprises a trigger, a rule, or both.

32. The method of claim 31, further comprising determining, by the data processor, the recommendation based on the trigger, the rule, or both.

33. The method of claim 31, wherein the at least one selected recommendation template is a pre-defined template or a custom template.

34. The method of claim 31, wherein the at least one selected recommendation template is determined by a machine-learning algorithm.

35. The method of claim 25, further comprising confirming, by the data processor, an access of the recommendation to the user, a third party, or both.

36. The method of claim 35, further comprising transmitting, by the data processor, of the recommendation to the user, the one or more service agents, or both is based on the confirmation of access.

37. The method of claim 25, further comprising transmitting, by the mobile processor, the recommendation to a service agent.

38. The method of claim 37, wherein the transmission is based on the confirmation of access.

39. The method of claim 25, wherein determining, by the data processor, the recommendation is performed by a machine learning algorithm.

40. The method of claim 25, wherein the user biological data comprises a weight, blood pressure, height, heart rate, food intake, nutritional history, activity history, sleep history, geolocation, body temperature, step count, body fat percentage, an emergency contact, a family contact, a friend contact, genetic data, genomic data, epigenetic information, microbiome information, proteomic and transcriptomic information, immune repertoire information, pharmacogenetics, blood oxygen levels, travel information, or drug-drug interactions, or any combination thereof.

41. The method of claim 25, wherein the recommendation comprises a fitness recommendation, nutrition recommendation, mental health recommendation, a recommendation for further testing, or any combination thereof.

42. A computer-readable storage medium comprising instructions executable by at least one processor, the instructions comprising the steps of claim 25.
